(19) United States
(12) Patent Application Publication
(10) Pub. No.: US 2009/0175847 A1
(43) Pub. Date: Jul. 9, 2009

(54) HUMANIZED ANTIBODIES TO AB (20-42) GLOBULOMER AND USES THEREOF

(75) Inventors: Stefan Barghorn, Mannheim (DE); Ulrich Ebert, Mannheim (DE); Heinz Hillen, Hassloch (DE); Patrick Keller, Darmstadt (DE); Andreas R. Striebinger, Speyer (DE); Boris Labkovsky, Wales, MA (US); Paul R. Hinton, Sunnyvale, CA (US); Veronica M. Juan, Menlo Park, CA (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, ABBOTT PARK, IL (US)

(21) Appl. No.: 12/129,469

(22) Filed: May 29, 2008

Related U.S. Application Data
(60) Provisional application No. 60/940,932, filed on May 30, 2007.
(60) Provisional application No. 60/990,359, filed on Nov. 27, 2007.

(51) Int. Cl. A61K 39/395; C07K 16/00; C12N 15/74; C12N 5/02; C12P 21/00; G01N 33/53; A61P 25/28

(52) U.S. Cl. 424/130.1; 530/387.1; 435/320.1; 435/325; 435/69.1; 435/7.1

(57) ABSTRACT

The present invention relates to binding proteins and, in particular, humanized antibodies that may be used, for example, in the diagnosis, treatment and prevention of Alzheimer's Disease and related conditions.

The subject application claims priority to pending U.S. Provisional Patent Application Ser. No. 60/940,932 filed on May 30, 2007 and pending U.S. Provisional Application Ser. No. 60/990,359 filed on Nov. 27, 2007, which are hereby incorporated in their entirety by reference.

### REFERENCE TO JOINT RESEARCH AGREEMENT

Contents of this application are under a joint research agreement entered into by and between Protein Design Labs, Inc. and Abbott Laboratories on Aug. 31, 2006, and directed to humanized amyloid beta antibodies.

### CROSS-REFERENCE TO RELATED APPLICATIONS

The subject application is related to pending International Appln. No. PCT/US2006/046148 filed on Nov. 30, 2006.

### BACKGROUND OF THE INVENTION

1. Field of the Invention

The present invention relates to antibodies that may be used, for example, in the diagnosis, treatment and prevention of Alzheimer's Disease and related conditions.

2. Background Information

Alzheimer's Disease (AD) is a neurodegenerative disorder characterized by a progressive loss of cognitive abilities and by characteristic neuropathological features comprising amyloid deposits, neurofibrillary tangles and neuronal loss in several regions of the brain (see Hardy and Selkoe (Science 297, 353 (2002); Mattson Nature 431, 7004 (2004). The principal constituents of amyloid deposits are amyloid beta-peptides (Aβ), with the 42 amino acid-long type Aβ(1-42) being the most prominent.

In particular, amyloid β(1-42) protein is a polypeptide having 42 amino acids which is derived from the amyloid precursor protein (APP) by proteolytic processing. This also includes, in addition to human variants, isoforms of the amyloid β(1-42) protein present in organisms other than humans, in particular, other mammals, especially rats. This protein, which tends to polymerize in an aqueous environment, may be present in very different molecular forms.

A simple correlation of the deposition of insoluble protein with the occurrence or progression of dementia disorders such as, for example, Alzheimer's disease, has proved to be unconvincing (Terry et al., Ann. Neurol. 30. 572-580 (1991); Dickson et al., Neurobiol. Aging 16, 285-298 (1995)). In contrast, the loss of synapses and cognitive perception seems to correlate better with soluble forms of Aβ(1-42)(Lue et al., Am. J. Pathol. 155, 853-862 (1999); McLean et al., Ann. Neurol. 46, 860-866 (1999)).

Although polyclonal and monoclonal antibodies have been raised in the past against Aβ(1-42), none have proven to produce the desired therapeutic effect without also causing serious side effects in animals and/or humans. For example, passive immunization results from preclinical studies in very old APP23 mice which received a N-terminal directed anti-Aβ(1-42) antibody once weekly for 5 months indicate therapeutically relevant side effects. In particular, these mice showed an increase in number and severity of microhemorrhages compared to saline-treated mice (Pfeifer et al., Science 2002 298:1379). A similar increase in hemorrhages was also described for very old (>24 months) Tg2576 and PDAPP mice (Wilcock et al., J Neuroscience 2003, 23: 3745-51; Racke et al., J Neuroscience 2005, 25:629-636). In both strains, injection of anti-Aβ(1-42) resulted in a significant increase of microhemorrhages. Thus, a tremendous, unmet therapeutic need exists for the development of biologics that prevent or slow down the progression of the disease without inducing negative and potentially lethal effects on the human body. Such a need is particularly evident in view of the increasing longevity of the general population and, with this increase, an associated rise in the number of patients annually diagnosed with Alzheimer's Disease or related disorders. Further, such antibodies will allow for proper diagnosis of Alzheimer's Disease in a patient experiencing symptoms thereof, a diagnosis which can only be confirmed upon autopsy at the present time. Additionally, the antibodies will allow for the elucidation of the biological properties of the proteins and other biological factors responsible for this debilitating disease.

All patents and publications referred to herein are hereby incorporated in their entirety by reference.

### SUMMARY OF THE INVENTION

The present invention pertains to binding proteins, particularly humanized antibodies (e.g., those referred to interchangeably herein as “humanized 7C6” or “7C6hum7 wt” for the humanized 7C6 antibody with a wildtype IgG1 constant region and “7C6hum7mut” for the humanized 7C6 antibody with a mutated IgG1 constant region and those referred to interchangeably herein as “humanized 5F7”, and “5F7hum8” for the humanized 7C6 antibody with a wildtype IgG1 constant region and “5F7hum8mut”) capable of binding to soluble oligomers and, for example, Aβ(20-42) globulomer present in the brain of a patient having Alzheimer's Disease. It is noted that the antibodies of the present invention may also be reactive with (i.e. bind to) Aβ forms other than the Aβ globulomers described herein. These antigens may or may not be oligomeric or globulomeric. Thus, the antigens to which the antibodies of the present invention bind include any Aβ form that comprises the globulomer epitope with which the antibodies of the present invention are reactive. Such Aβ forms include truncated and non-truncated Aβ(X-Y) forms (with X and Y being defined as herein), such as Aβ(20-42), Aβ(20-40), Aβ(12-42), Aβ(12-40), Aβ(1-42), and Aβ(1-40) forms, provided that said forms comprise the globulomer epitope. Further, the present invention also provides methods of producing and using these binding proteins or portions thereof.

In particular, the subject invention encompasses a binding protein comprising an antigen binding domain which binds to amyloid-beta (20-42) globulomer, said antigen binding domain comprising at least one CDR comprising an amino acid sequence selected from the group consisting of:
CDR-VH1. X₁-X₂-X₃-X₄-X₅-X₆-X₇ (SEQ ID NO.:5), wherein:
   X₁ is T or S;
   X₂ is F or Y;
   X₃ is Y or A;
   X₄ is I or M; and
   X₅ is H or S.
CDR-VH2. X₁-X₂-X₃-X₄-X₅-X₆-X₇-X₈-X₉-X₁₀-Xₗₗ-X₁₂-X₁₃-X₁₄-X₁₅-X₁₆-X₁₇ (SEQ ID NO.:6), wherein:
   X₁ is M or S;
   X₂ is I;
   X₃ is G or H;
   X₄ is P or N;
   X₅ is G or R;
   X₆ is S or G;
   X₇ is G or T;
   X₈ is N or I;
   X₉ is T or F;
   X₁₀ is Y;
   X₁₁ is Y or L;
   X₁₂ is N or D;
   X₁₃ is E or S;
   X₁₄ is M or V;
   X₁₅ is F or K;
   X₁₆ is K or G; and
   X₁₇ is D or is not present.
CDR-VH3. X₁-X₂-X₃-X₄-X₅-X₆-X₇-X₈-X₉-X₁₀-X₁₁-X₁₂-X₁₃ (SEQ ID NO.:7), wherein:
   X₁ is A or G;
   X₂ is K or R;
   X₃ is S;
   X₄ is A or N;
   X₅ is R or S;
   X₆ is A or Y;
   X₇ is A;
   X₈ is W or M;
   X₉ is F or D;
   X₁₀ is A or Y; and
   X₁₁ is Y or is not present.
CDR-VL1. X₁-X₂-X₃-X₄-X₅-X₆-X₇-X₈-X₉-X₁₀-X₁₁-X₁₂-X₁₃-X₁₄-X₁₅-X₁₆ (SEQ ID NO.:8), wherein:
   X₁ is R;
   X₂ is S
   X₃ is S or T;
   X₄ is Q;
   X₅ is S or T;
   X₆ is V or L;
   X₇ is V;
   X₈ is Q or H;
   X₉ is S or R;
   X₁₀ is N;
   X₁₁ is G;
   X₁₂ is N or D;
   X₁₃ is T;
   X₁₄ is Y;
   X₁₅ is N or L and
   X₁₆ is E.
CDR-VL2. X₁-X₂-X₃-X₄-X₅-X₆-X₇-X₈ (SEQ ID NO.:9), wherein:
   X₁ is K;
   X₂ is V;
   X₃ is S;
   X₄ is N;
   X₅ is R;
   X₆ is F; and
   X₇ is S.
and
CDR-VL3. X₁-X₂-X₃-X₄-X₅-X₆-X₇-X₈-X₉ (SEQ ID NO.:10), wherein:
   X₁ is F;
   X₂ is Q;
   X₃ is G;
   X₄ is S;
   X₅ is H;
   X₆ is V;
   X₇ is P;
   X₈ is P or Y; and
   X₉ is T.
      This binding protein has a binding affinity to the amyloid beta (20-42) globulomer which is greater than to at least one amyloid beta peptide or protein selected from the group consisting of an amyloid beta (1-42) globulomer, an amyloid beta (12-42) globulomer, an s-amyloid precursor protein, an amyloid beta (1-40) monomer, an amyloid beta (1-42) monomer and an amyloid beta (1-42) fibril.

One aspect of this invention pertains to a binding protein (e.g., antibody) comprising an antigen binding domain capable of binding to an Aβ(20-42) globulomer or any other Aβ form that comprises the globulomer epitope with which the antibodies of the present invention are reactive. In one embodiment, the antigen-binding domain comprises at least one CDR comprising an amino acid sequence selected from the group consisting of: residues 30-35 (i.e., TFYIH (SEQ ID NO.:11); 5F7 VH CDR1) of SEQ ID NO.:1; residues 50-66 (i.e., MIGPGSGNTYYNEMFKD (SEQ ID NO.:12); 5F7 VH CDR2) of SEQ ID NO.:1; residues 98-108 (i.e., AKSARAAWFAY (SEQ ID NO.:13); 5F7 VH CDR3) of SEQ ID NO.:1; residues 24-39 (i.e., RSSQSVVQSNGNTYLE (SEQ ID NO.:14); 5F7 VL CDR1) of SEQ ID NO.:2; residues 55-61 (i.e., KVSNRFS (SEQ ID NO.:15); 5F7 VL CDR2) of SEQ ID NO.:2; residues 94-102 (i.e., FQGSHVPPT (SEQ ID NO.:65); 5F7 VL CDR3) of SEQ ID NO.:2; residues 31-35 (i.e., SYAMS (SEQ ID NO.:16); 7C6 VH CDR1) of SEQ ID NO.:3; residues 50-65 (i.e., SIHNRGTIFYLDSVKG (SEQ ID NO.:17); 7C6 VH CDR2) of SEQ ID NO.:3; residues 98-107 (i.e., GRSNSYAMDY (SEQ ID NO.:18); 7C6 VH CDR3) of SEQ ID NO.:3; residues 24-39 (i.e., RSTQTLVHRNGDTYLE (SEQ ID NO.:19); 7C6 VL CDR1) of SEQ ID NO.:4; residues 55-61 (i.e., KVSNRFS (SEQ ID NO.:20); 7C6 VL CDR2) of SEQ ID NO.:4; residues 94-102 (i.e., FQGSHVPYT (SEQ ID NO.:21); 7C6 VL CDR3) of SEQ ID NO.:4. In a preferred embodiment, the binding protein comprises at least 3 CDRs selected from the group consisting of the sequences disclosed above. More preferably, the 3 CDRs selected are from sets of variable domain CDRs selected from the group consisting of:

TABLE 1

| | |
|---|---|
| VH 5F7hum8 CDR Set | |
| VH 5F7 CDR-H1 | Residues 31-35 of SEQ ID NO.: 1 |
| VH 5F7 CDR-H2 | Residues 50-66 of SEQ ID NO.: 1 |
| VH 5F7 CDR-H3 | Residues 98-108 of SEQ ID NO.: 1 |
| VL 5F7 hum8 CDR Set | |
| VL 5F7 CDR-L1 | Residues 24-39 of SEQ ID NO.: 2 |
| VL 5F7 CDR-L2 | Residues 55-61 of SEQ ID NO.: 2 |
| VL 5F7 CDR-L3 | Residues 94-102 of SEQ ID NO.: 2 |
| VH 7C6 hum7 CDR Set | |
| VH 7C6 CDR-H1 | Residues 31-35 of SEQ ID NO.: 3 |
| VH 7C6 CDR-H2 | Residues 50-65 of SEQ ID NO.: 3 |
| VH 7C6 CDR-H3 | Residues 98-107 of SEQ ID NO.: 3 |
| VL 7C6 hum7 CDR Set | |
| VL 7C6 CDR-L1 | Residues 24-39 of SEQ ID NO.: 4 |
| VL 7C6 CDR-L2 | Residues 55-61 of SEQ ID NO.: 4 |
| VL 7C6 CDR-L3 | Residues 94-102 of SEQ ID NO.: 4 |

In one embodiment, the binding protein of the invention comprises at least two variable domain CDR sets. More preferably, the two variable domain CDR sets are selected from a group consisting of: VH 5F7 CDR Set & VL 5F7 CDR Set and VH 7C6 CDR Set & VL 7C6 CDR Set.

In another embodiment the binding protein disclosed above further comprises a human acceptor framework. Preferably the human acceptor framework comprises an amino acid sequence selected from the group consisting of:

| | |
|---|---|
| QVQLVQSGAEVKKPGASVKVSCKASGYTFT; | (SEQ ID NO.:22) |
| WRQAPGQGLEWMG; | (SEQ ID NO.:23) |
| RVTMTRDTSTSTVYMELSSLRSEDTAVYYCAR; | (SEQ ID NO.:24) |
| WGQGTLVTVSS; | (SEQ ID NO.:25) |
| DIVMTQSPLSLPVTPGEPASISC; | (SEQ ID NO.:26) |
| WYLQKPGQSPQLLIY; | (SEQ ID NO.:27) |
| GVPDRFSSGSGTDFTLKISRVEAEDVGVYYC; | (SEQ ID NO.:28) |
| FGGGTKVEIKR | (SEQ ID NO.:29) |
| EVQLVESGGGLVKPGGSLRLSCAASGFTFS; | (SEQ ID NO.:30) |
| WVRQAPGKGLEWVS; | (SEQ ID NO.:31) |
| RFTISRDNAKNSLYLQMNSLRAEDTAVYYCAR; | (SEQ ID NO.:32) |
| WGQGTLVTVSS; | (SEQ ID NO.:33) |
| DIVMTQSPLSLPVTPGEPASISC; | (SEQ ID NO.:34) |
| WYLQKPGQSPQLLIY; | (SEQ ID NO.:35) |
| GVPDRFSGSGSGTDFTLKISRVEAEDVGVYYC; | (SEQ ID NO.:36) |
| and | |
| FGQGTKLEIKR. | (SEQ ID NO.:37) |

In a preferred embodiment, the binding protein is a humanized antibody or antigen binding portion thereof capable of binding to an Aβ(20-42) globulomer and/or to any Aβ form that comprises the globulomer epitope with which the antibodies of the present invention are reactive. Preferably, the humanized antibody or antigen binding portion thereof comprises one or more CDRs disclosed above (see Table 5 below). More preferably, the humanized antibody or antigen binding portion thereof comprises at least one variable domain having an amino acid sequence selected from the group consisting of SEQ ID NO.:23, SEQ ID NO.:24, SEQ ID NO.:25 and SEQ ID NO.:26. Most preferably, the humanized antibody or antigen binding portion thereof comprises two variable domains selected from the group disclosed above. Preferably, the humanized antibody or antigen binding portion thereof comprises a human acceptor framework. More preferably, the human acceptor framework is any one of the human acceptor frameworks disclosed above.

In a preferred embodiment, the binding protein is a humanized antibody or antigen binding portion thereof capable of binding an Aβ(20-42) globulomer and/or to any Aβ form that comprises the globulomer epitope with which the antibodies of the present invention are reactive. Preferably, the humanized antibody or antigen binding portion thereof comprises one or more CDRs disclosed above incorporated into a human antibody variable domain of a human acceptor framework. Preferably, the human antibody variable domain is a consensus human variable domain. More preferably, the human acceptor framework comprises at least one Framework Region amino acid substitution at a key residue, wherein the key residue is selected from the group consisting of a residue adjacent to a CDR; a glycosylation site residue; a rare residue; a residue capable of interacting with an Aβ (20-42) globulomer; a residue capable of interacting with a CDR; a canonical residue; a contact residue between heavy chain variable region and light chain variable region; a residue within a Vernier zone; and a residue in a region that overlaps between a Chothia-defined variable heavy chain CDR1 and a Kabat-defined first heavy chain framework. Preferably, the human acceptor framework human acceptor framework comprises at least one Framework Region amino acid substitution, wherein the amino acid sequence of the framework is at least 65% identical to the sequence of said human acceptor framework and comprises at least 70 amino acid residues identical to said human acceptor framework.

In a preferred embodiment, the binding protein is a humanized antibody or antigen binding portion thereof capable of binding to an Aβ(20-42) globulomer and/or any Aβ form that comprises the globulomer epitope with which the antibodies of the present invention are reactive.

It is noted, again, that the antibodies of the present invention may also be reactive with, i.e. bind to, Aβ forms other than the Aβ globulomers described herein. These antigens may or may not be oligomeric or globulomeric. Thus, the antigens to which the antibodies of the present invention bind include any Aβ form that comprises the globulomer epitope with which the antibodies of the present invention are reactive. Such Aβ forms include truncated and non-truncated Aβ(X-Y) forms (with X and Y being defined as above), such as Aβ(20-42), Aβ(20-40), Aβ(12-42), Aβ(12-40), Aβ(1-42), and Aβ(1-40) forms, provided that these forms comprise the globulomer epitope.

Preferably, the humanized antibody, or antigen binding portion, thereof comprises one or more CDRs disclosed above. More preferably, the humanized antibody, or antigen binding portion thereof, comprises three or more CDRs disclosed above. Most preferably the humanized antibody, or antigen-binding portion thereof, comprises six CDRs disclosed above.

In another embodiment of the claimed invention, the humanized antibody or antigen binding portion thereof comprises at least one variable domain having an amino acid sequence selected from the group consisting of SEQ ID NO.:1, SEQ ID NO.:2, SEQ ID NO.:3 and SEQ ID NO.:4. With respect to SEQ ID NO.:1 (5F7 VL), based upon Kabat numbering, amino acid position 1 may be E or Q; position 5 may be V or K; position 11 may be V or L; position 12 may be K or V; position 13 may be K or R; position 16 may be A or T; position 20 may be V or M; position 38 may be R or K; position 40 may be A or R; position 75 may be T or S; position 81 may be E or Q; position 83 may be R or T; position 87 may be T or S; and position 91 may be Y or F. In connection with SEQ ID NO.:2 (5F7 VH), based upon Kabat numbering, amino acid position 2 may be I or V; position 3 may be V or L; position 7 may be S or T; position 14 may be T or S; position 15 may be P or L; position 17 may be E or D; position 18 may be P or Q; position 45 may be Q or K; and position 83 may be V or L. With respect to SEQ ID NO.:3 (7C6 VH), amino acid position 19 may be R or K; position 40 may be A or T; position 42 may be G or A; position 44 may be G or R; position 82A may be N or S; position 84 may be L or S; and position 89 may be V or I. With regard to SEQ ID NO.:4 (7C6 VL), based upon Kabat numbering, amino acid position 14 may be T or R; position 15 may be P or L; position 17 may be E or D; position 18 may be P or Q; position 45 may be Q or K; and position 83 may be V or L. More preferably, the humanized antibody or antigen-binding portion thereof comprises two variable domains selected from the group disclosed above. Most preferably, humanized antibody, or an antigen-binding portion thereof, comprises two variable domains, wherein said two variable domains have amino acid sequences selected from the group consisting of (SEQ ID NO.:1 & SEQ ID NO.:2) and (SEQ ID NO.:3 & SEQ ID NO.:4).

In a preferred embodiment, the binding protein disclosed above comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain. More preferably, the binding protein comprises SEQ ID NO.:38, SEQ ID NO.:39, SEQ ID NO.:40 and SEQ ID NO.:41.

In a more preferred embodiment, the binding protein disclosed above comprises a mutated heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain. Mutations of heavy chain constant regions that modulate effector functions or antibody halflife are well recognized in the art (Boris, add refs.).

In an even more preferred embodiment, the binding protein disclosed above comprises a wiltype or mutated heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain and a lambda or kappa light chain.

In an even more preferred embodiment, the binding protein disclosed above comprises a wiltype or mutated heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain and a kappa light chain. The binding protein of the invention is capable of binding Aβ(20-42) globulomer and may also bind any Aβ form that comprises the globulomer epitope with which the antibodies of the present invention are reactive. Preferably, the binding protein is capable of modulating a biological function of an Aβ(20-42) globulomer. More preferably, the binding protein is capable of neutralizing an Aβ(20-42) globulomer.

In another embodiment, the binding protein of the invention has a dissociation constant (K_{D}) to an Aβ(20-42) globulomer in the range of 1×10⁻⁶ M to 1×10⁻¹² M. Preferably, the antibody binds to an Aβ(20-42) globulomer with high affinity, for example, with a K_{D} of about 1×10⁻⁷ or greater, with a K_{D} of about 1×10⁻⁸ or greater, with a K_{D} of about 1×10⁻⁹ or greater, with a K_{D} of about 1×10⁻¹⁰ or greater, or with a K_{D} of about 1×10⁻¹¹ M or greater.

It is preferred that the binding affinity of the antibody to the Aβ(20-42) globulomer is at least 2 times (e.g., at least 3 or at least 5 times), preferably at least 10 times (e.g., at least 20 times, at least 30 times or at least 50 times), more preferably at least 100 times (e.g., at least 200 times, at least 300 times or at least 500 times), and even more preferably at least 1000 times (e.g., at least 2000 times, at least 3000 times or at least 5000 times), even more preferably at least 10,000 times (e.g., at least 20,000 times, at least 30,000 times or at least 50,000 times), and most preferably at least 100,000 times greater than the binding affinity of the antibody to the Aβ(12-42) globulomer or to the Aβ (1-42) globulomer. Further, the affinity of the antibody to the Aβ(20-42) globulomer should be greater than its affinity to both the Aβ(1-40) monomer and the Aβ(1-40) monomer.

One embodiment of the invention provides an antibody construct comprising any one of the binding proteins disclosed above and a linker polypeptide or an immunoglobulin. In a preferred embodiment, the antibody construct is selected from the group consisting of an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, a bispecific antibody or a Dual Variable Domain (DVD) binding molecule. In a preferred embodiment, the antibody construct comprises a heavy chain immunoglobulin constant domain selected from the group consisting of a human IgM constant domain, a human IgG1 constant domain, a human IgG2 constant domain, a human IgG3 constant domain, a human IgG4 constant domain, a human IgE constant domain, and a human IgA constant domain. More preferably, the antibody construct comprises (SEQ ID NO.:38 and SEQ ID NO.:39) or (SEQ ID NO.:40 and SEQ ID NO.:41). In another embodiment, the invention provides an antibody conjugate comprising an the antibody construct disclosed above and an agent an agent selected from the group consisting of an immunoadhesion molecule, an imaging agent, a therapeutic agent, and a cytotoxic agent. In a preferred embodiment the imaging agent selected from the group consisting of a radiolabel, an enzyme, a fluorescent label, a luminescent label, a bioluminescent label, a magnetic label, and biotin. More preferably the imaging agent is a radiolabel selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm. In a preferred embodiment the therapeutic or cytotoxic agent is selected from the group consisting of an anti-metabolite, an alkylating agent, an antibiotic, a growth factor, a cytokine, an anti-angiogenic agent, an anti-mitotic agent, an anthracycline, toxin, and an apoptotic agent.

In another embodiment the antibody construct is glycosylated. Preferably, the glycosylation is a human glycosylation pattern.

In another embodiment, the binding protein, antibody construct or antibody conjugate disclosed above exists as a crystal. Preferably, the crystal is a carrier-free pharmaceutical controlled release crystal. In a preferred embodiment, the crystallized binding protein, crystallized antibody construct or crystallized antibody conjugate has a greater half life in vivo than its soluble counterpart. In another preferred embodiment, the crystallized binding protein, crystallized antibody construct or crystallized antibody conjugate retains biological activity after crystallization.

One aspect of the invention pertains to an isolated nucleic acid molecule encoding the binding protein, antibody construct or antibody conjugate disclosed above. A further embodiment provides a vector comprising the isolated nucleic acid disclosed above wherein said vector is selected from the group consisting of pcDNA; pTT (Durocher et al., Nucleic Acids Research 2002, Vol 30, No. 2); pTT3 (pTT with additional multiple cloning site; pEFBOS (Mizushima, S. and Nagata, S., (1990) Nucleic acids Research Vol 18, No. 17); pBV; pJV; and pBJ.

In another aspect, a host cell is transformed with the vector disclosed above. Preferably, the host cell is a prokaryotic cell. More preferably, the host cell is E. coli. In a related embodiment, the host cell is an eukaryotic cell. Preferably, the eukaryotic cell is selected from the group consisting of a protist cell, an animal cell, a plant cell and a fungal cell. More preferably, the host cell is a mammalian cell including, but not limited to, CHO and COS; or a fungal cell such as Saccharomyces cerevisiae; or an insect cell such as Sf9.

Another aspect of the invention provides a method of producing a binding protein that binds Aβ(20-42) globulomer and/or any other Aβ form that comprises the globulomer epitope with which the antibodies of the present invention are reactive, comprising culturing any one of the host cells disclosed above in a culture medium under conditions and for a time sufficient to produce a binding protein that binds Aβ(20-42) and/or any other Aβ form that comprises the globulomer epitope with which the antibodies of the present invention are reactive. Another embodiment provides a binding protein produced according to the method disclosed above and/or any other Aβ form that comprises the globulomer epitope with which the antibodies of the present invention are reactive.

One embodiment provides a composition for the release of a binding protein, as defined herein, wherein the composition comprises a formulation which in turn comprises a crystallized binding protein, crystallized antibody construct or crystallized antibody conjugate as disclosed above and an ingredient; and at least one polymeric carrier. Preferably, the polymeric carrier is a polymer selected from one or more of the group consisting of: poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly ((hydroxypropyl) methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polyeaccharides, blends and copolymers thereof. Preferably the ingredient is selected from the group consisting of albumin, sucrose, trehalose, lactitol, gelatin, hydroxypropyl-cyclodextrin, methoxypolyethylene glycol and polyethylene glycol. Another embodiment provides a method for treating a mammal comprising the step of administering to the mammal an effective amount of the composition disclosed above.

The invention also provides a pharmaceutical composition comprising a binding protein, antibody construct or antibody conjugate as disclosed above and a pharmaceutically acceptable carrier. In a further embodiment, the pharmaceutical composition comprises at least one additional therapeutic agent for treating a disorder in which activity is detrimental. Preferably the additional agent is selected from the group consisting of: a monoclonal antibody (e.g., a TNF antagonist such as, for example, Remicade and Humira®), a TNF receptor fusion protein (e.g., Enbrel), a polyclonal antibody, a fragment of a monoclonal antibody, a cholesterinase inhibitor, a partial NMDA receptor blocker, a glycosaminoglycan mimetic, an inhibitor or allosteric modulator of gamma secretase, a luteinizing hormone blockade gonadotropin releasing hormone agonist, a serotinin 5-HT1A receptor antagonist, a chelating agent, a neuronal selective L-type calcium channel blocker, an immunomodulator, an amyloid fibrillogenesis inhibitor or amyloid protein deposition inhibitor, a 5-HT1a receptor antagonist, a PDE4 inhibitor, a histamine agonist, a receptor protein for advanced glycation end products, a PARP stimulator, a serotonin 6 receptor antagonist, a 5-HT4 receptor agonist, a human steroid, a glucose uptake stimulant which enhances neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator.

In another aspect, the invention provides a method for inhibiting activity of Aβ(20-42) globulomer (or any other Aβ form that comprises the globulomer epitope with which the antibodies of the present invention are reactive), comprising contacting Aβ(20-42) globulomer (or other Aβ form comprising the globulomer epitope with which the antibody is reactive), as appropriate, with a binding protein disclosed above such that Aβ(20-42) globulomer activity (or other amyloid beta protein form) is inhibited. In a related aspect, the invention provides a method for inhibiting human Aβ(20-42) globulomer activity (or any other Aβ form that comprises the globulomer epitope with which the antibodies of the present invention are reactive) in a human subject suffering from a disorder in which Aβ(20-42) globulomer activity (or activity of other Aβ form comprising the globulomer epitope with which the antibodies of the present invention are reactive) is detrimental, comprising administering to the human subject a binding protein disclosed above such that Aβ(20-42) globulomer activity (or activity of other Aβ form comprising the globulomer epitope with which the antibodies are reactive) in the human subject is inhibited and treatment is achieved. Preferably, the disorder is selected from an amyloidosis such as, for example, Alzheimer's Disease or Down's Syndrome.

In another aspect, the invention provides a method of treating a patient suffering from a disorder in which Aβ(20-42) globulomer is detrimental (or other detrimental Aβ form comprising the globulomer epitope with which the antibody reacts) comprising the step of administering any one of the binding proteins disclosed above before, concurrent, or after the administration of a second agent, as described above. In a preferred embodiment, the second agent is selected from the group consisting of a small molecule or a biologic such as those listed above.

In a preferred embodiment the pharmaceutical compositions disclosed above are administered to the subject by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

One aspect of the invention provides at least one Aβ(20-42) globulomer anti-idiotype antibody to at least one Aβ(20-42) globulomer binding protein of the present invention and/or any other Aβ form that comprises the globulomer epitope with which the antibodies of the present invention are reactive. The anti-idiotype antibody includes any protein or peptide containing molecule that comprises at least a portion of an immunoglobulin molecule such as, but not limited to, at least one complementarity determining region (CDR) of a heavy or light chain or a ligand binding portion thereof, a heavy chain or light chain variable region, a heavy chain or light chain constant region, a framework region, or any portion of any one of these entities that can be incorporated into a binding protein of the present invention.

### BRIEF DESCRIPTION OF THE FIGURES

FIG. 1(A) illustrates the nucleotide sequence (SEQ ID NO.:42) of the variable heavy chain of humanized antibody 5F7 (i.e., 5F7 VH (hum8)), and FIG. 1(B) illustrates the amino acid sequence (SEQ ID NO.:1) of the variable heavy chain of humanized antibody 5F7. FIG. 1(C) illustrates the nucleotide sequence (SEQ ID NO.:43) of the variable light chain of humanized antibody 5F7 (i.e., 5F7 VL (hum 8)), and FIG. 1(D) illustrates the amino acid sequence (SEQ ID NO.:2) encoded by this nucleotide sequence. (All CDR regions are underlined in the figures.)

FIG. 2(A) illustrates the nucleotide sequence (SEQ ID NO.:44) of the variable heavy chain of humanized antibody 7C6 (i.e., 7C6 VH (hum7)), and FIG. 2(B) illustrates the amino acid sequence (SEQ ID NO.:3) of the variable heavy chain of humanized antibody 7C6. FIG. 2(C) illustrates the nucleotide sequence (SEQ ID NO.:45) of the variable light chain of humanized antibody 7C6 (i.e., 7C6 VL (hum 7)), and FIG. 2(D) illustrates the amino acid sequence (SEQ ID NO.:4) encoded by this nucleotide sequence. (All CDR regions are underlined in the figures.)

FIG. 3 illustrates the binding of the biotinylated mouse 5F7 to the truncated 20-42 globulomer. In particular, binding of the biotinylated mouse 5F7 antibody is inhibited by increasing amounts of unlabeled mouse 5F7 (“HYB”) or humanized antibody 5F7 (“HUM8).

FIG. 4 illustrates the binding of the biotinylated mouse 7C6 to the truncated 20-42 globulomer. Binding of the biotinylated mouse 7C6 antibody is inhibited by increasing amounts of unlabeled mouse antibody 7C6 (“HYB) and humanized antibody 7C6hum7 (“HUM7”).

FIG. 5(A) shows an SDS PAGE of standard proteins (molecular marker proteins, lane 1); Aβ(1-42) fibril preparation; control (lane 2); Aβ(1-42) fibril preparation+mAb 5F7hum8, 20 h, 37° C., supernatant (lane 3); Aβ(1-42) fibril preparation+mAb 5F7hum8, 20 h, 37° C., pellet (lane 4); Aβ(1-42) fibril preparation+mAb 7C6hum7mut, 20 h, 37° C., supernatant (lane 5); Aβ(1-42) fibril preparation+mAb 7C6hum7mut, 20 h, 37° C., pellet (lane 6); Aβ(1-42) fibril preparation+mAb 7C6hum7 wt, 20 h, 37° C., supernatant (lane 7); Aβ(1-42) fibril preparation+mAb 7C6hum7 wt, 20 h, 37° C., pellet (lane 8); Aβ(1-42) fibril preparation+mAb 6E10, 20 h, 37° C., supernatant (lane 9); Aβ(1-42) fibril preparation+mAb 6E10, 20 h 37° C., pellet (lane 10); Aβ (1-42) fibril preparation+mAb IgG2a, 20 h, 37° C., supernatant (lane 11); Aβ (1-42) fibril preparation+mAb IgG2a, 20 h, 37° C., pellet (lane 12); and FIG. 5(B) shows the results of the quantitative analysis of mAbs bound to Aβ-fibrils in percent of total antibody.

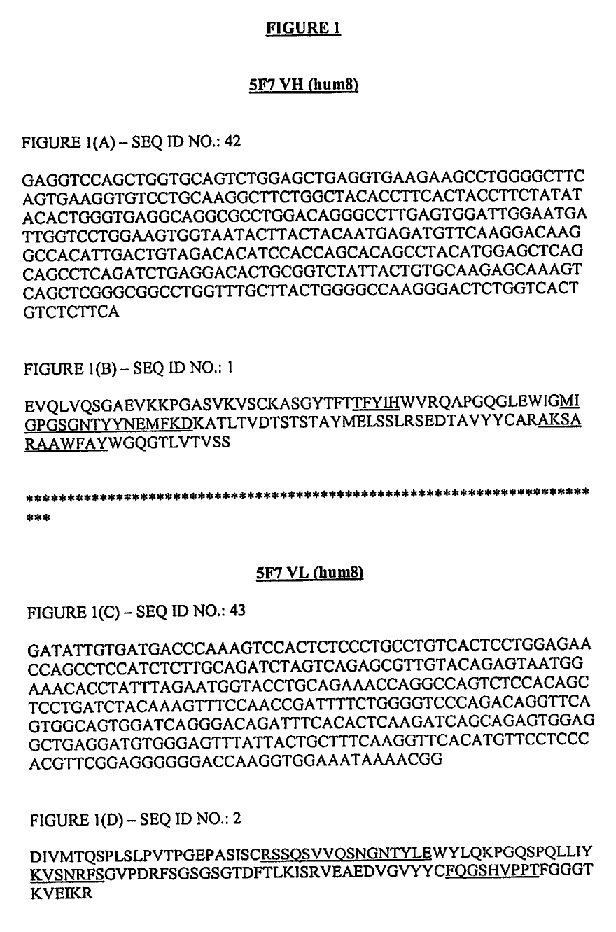

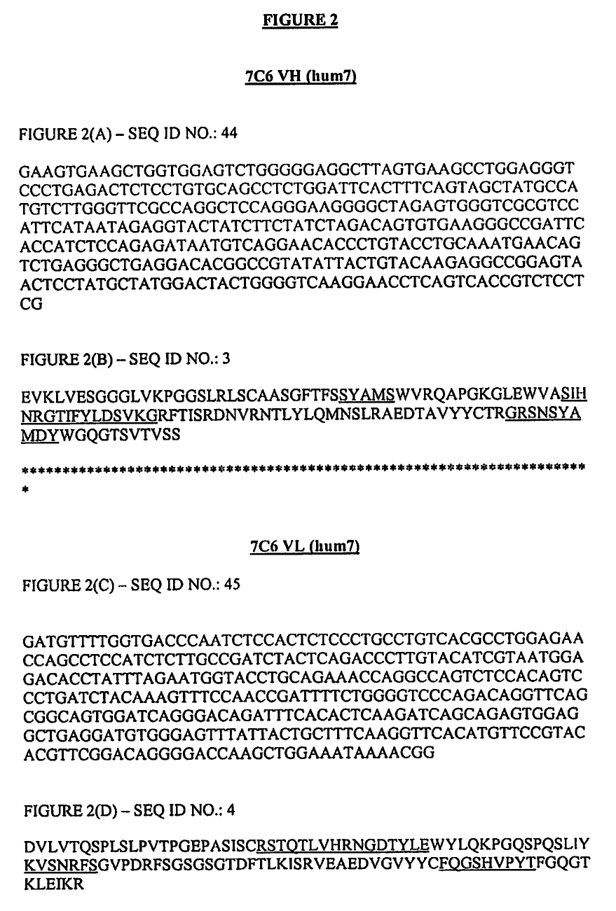

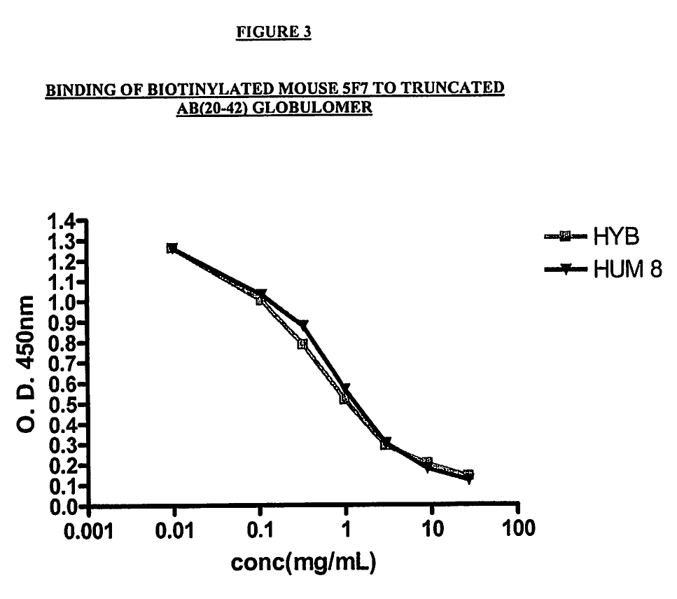

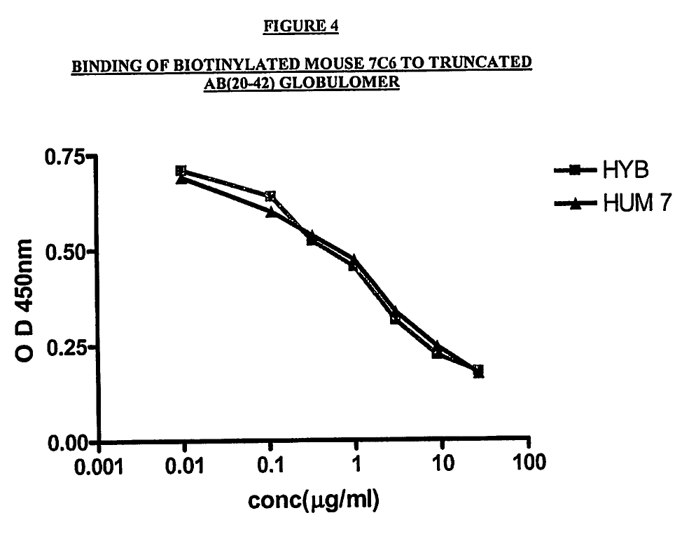

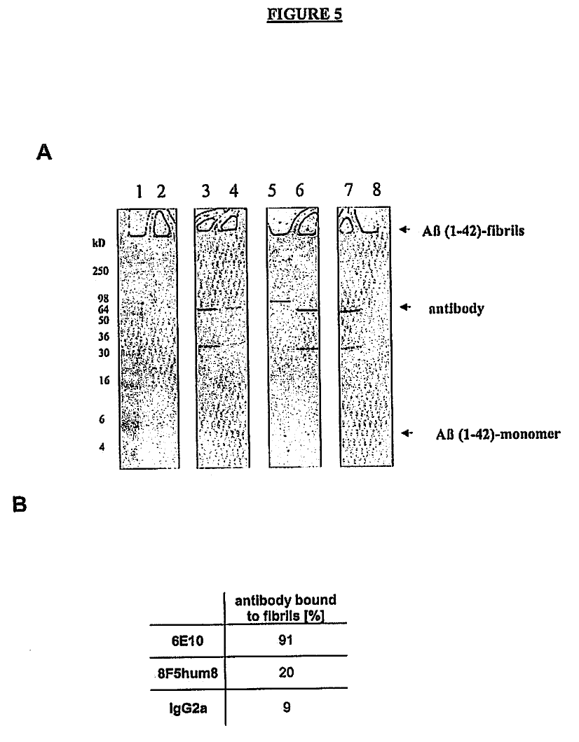

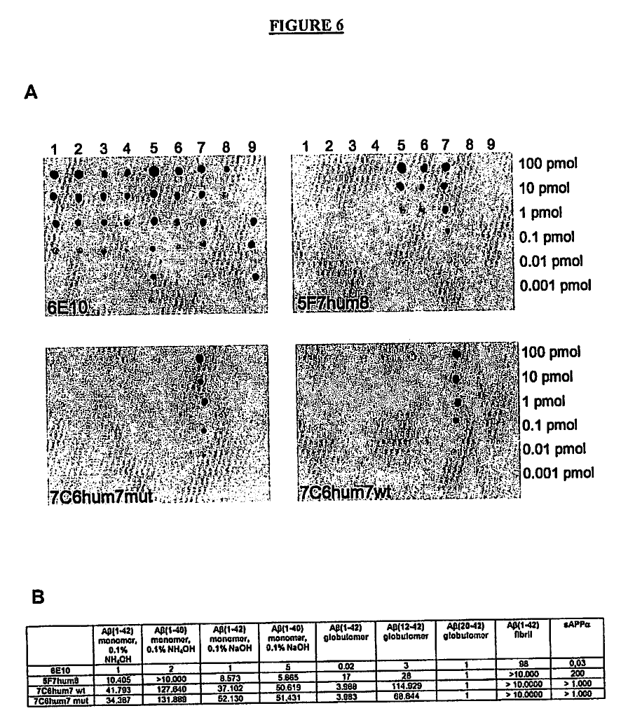

FIG. 6(A) shows a dot blot analysis of the specificity of different anti-Aβ antibodies (6E10, 5F7hum8, 7C6hum7 wt, 7C6hum7mut). The monoclonal antibodies tested here were obtained by active immunization of mice with Aβ(20-42) globulomer followed by selection of the fused hybridoma cells and subsequent humanization (except for the commercially available mouse monoclonal antibody 6E10, Signet No 9320). The individual Aβ forms were applied in serial dilutions and incubated with the respective monoclonal antibodies for immune reaction:
1. Aβ(1-42) monomer, 0.1% NH₄OH
2. Aβ(1-40) monomer, 0.1% NH₄OH
3. Aβ(1-42) monomer, 0.1% NaOH
4. Aβ(1-40) monomer, 0.1% NaOH
5. Aβ(1-42) globulomer
6. Aβ(12-42) globulomer
7. Aβ(20-42) globulomer
8. Aβ(1-42) fibril preparation
9. sAPPα (Sigma) (first dot: 1 pmol)

FIG. 6(B) illustrates the results obtained when quantitative evaluation was done using a densitometric analysis of the intensity. For each Aβ form, only the dot corresponding to the lowest antigen concentration was evaluated provided that it had a relative density of greater than 20% of the relative density of the last optically unambiguously identified dot of the Aβ(20-42) globulomer (threshold). This threshold value was determined for every dot-blot independently. The value indicates the relation between recognition of Aβ(20-42) globulomer and the respective Aβ form for the antibody given.

FIG. 7 illustrates the alignment of the 5F7VH region amino acid sequences. The amino acid sequences of 5F7VH (SEQ ID NO: 68), Hu5F7VH (SEQ ID NO: 69), and the human MUC1-1′ CL (SEQ ID NO: 70) and JH4 segments are shown in single letter code. The CDR sequences based on the definition of Kabat, E. A., et al. (1991) are underlined in the mouse 5F7VH sequence. The CDR sequences in the acceptor human VH segment are omitted in the figure. The single underlined amino acids in the Hu5F7VH sequence are predicted to contact the CDR sequences, and therefore have been substituted with the corresponding mouse residues. The double underlined amino acid in the Hu5F7VH sequence has been changed to the consensus amino acid in the same human VH subgroup to eliminate potential immunogenicity.

FIG. 8 illustrates the alignment of the 5F7VL region amino acid sequences. The amino acid sequences of 5F7VL (SEQ ID NO: 71), Hu5F7VL (SEQ ID NO: 72), and the human TR1.37′ CL (SEQ ID NO: 73) and JK4 segments are shown in single letter code. The CDR sequences based on the definition of Kabat, E. A., et al. (1991) are underlined in the mouse 5F7VL sequence. The CDR sequences in the acceptor human VL segment are omitted in the figure. The single underlined amino acid in the Hu5F7VL sequence is predicted to contact the CDR sequences, and therefore has been substituted with the corresponding mouse residue. The double underlined amino acids in the Hu5F7VL sequence have been changed to the consensus amino acids in the same human VL subgroup to eliminate potential immunogenicity.

FIG. 9 shows the binding of different antibodies to transverse sections of autopsy neocortices of two Alzheimer's disease patients and of 19 month old APP transgenic Tg2576 mice and 17 month old APP/Lo mice.

a) Staining of parenchymal deposits of Aβ (amyloid plaques; black arrows) and of vascular amyloid deposits (cerebral amyloid angiopathy, CAA; white arrows) at a concentration of 0.7 μg/ml occurs only with 6E10 and 4G8 but not with h7C6 wt and h7C6mut;
b) Quantification of the analysis of Aβ plaque staining by antibodies in the neocortex of the Alzheimer's disease patient RZ16 at a concentration of 0.7 μg/ml by histological image analysis. Optical density values (0%=surrounding background staining) were calculated from the greyscale values, and the differences between antibodies were statistically evaluated (ANOVA, F(3, 59)=207.7; P<0.0001; followed by posthoc Bonferroni's t-test): 6E10 and 4G8 were different from all other antibodies (P<0.001), while h7C6 wt and h7C6mut showed no staining at all.
c) Quantification of the analysis of Aβ plaque staining by antibodies in the neocortex of the Alzheimer's disease patient RZ55 at a concentration of 0.7 μg/ml by histological image analysis. Optical density values (0%=surrounding background staining) were calculated from the greyscale values, and the differences between antibodies were statistically evaluated (ANOVA, F(3, 59)=182.6, P<0.0001; followed by posthoc Bonferroni's t-test): 6E10 and 4G8 were different from all other antibodies (P<0.001), while h7C6 wt and h7C6mut showed no staining at all.
d) Quantification of the analysis of Aβ plaque staining by antibodies in the neocortex of the human APP_{Swedish} transgenic mouse line (Tg2576) at several concentrations by histological image analysis. Optical density values (0%=surrounding background staining) were calculated from the greyscale values, and the differences between antibodies at 0.7 μg/ml were statistically evaluated (ANOVA, F(3, 59)=290.9, P<0.0001; followed by posthoc Bonferroni's t-test): 6E10 and 4G8 were different from the h7C6 antibodies (P<0.001), while h7C6 wt and h7C6mut showed no staining at all.
e) Quantification of the analysis of Aβ plaque staining by antibodies in the neocortex of the human APP_{London} transgenic mouse line (APP/Lo) at several concentrations by histological image analysis. Optical density values (0%=surrounding background staining) were calculated from the greyscale values, and the differences between antibodies at 0.7 μg/ml were statistically evaluated (ANOVA, F(3, 50)=145.6, P<0.0001; followed by posthoc Bonferroni's t-test): 6E10 and 4G8 were different from the h7C6 antibodies (P<0.001), while h7C6 wt and h7C6mut showed no staining at all.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

In particular, the present invention provides globulomer-specific antibodies possessing high affinity for truncated forms of Aβ globulomers. These antibodies are capable of discriminating not only other forms of Aβ peptides, particularly monomers and fibrils, but also untruncated forms of Aβ globulomers. Thus, the present invention relates to an antibody having a binding affinity to an Aβ(20-42) globulomer that is greater than the binding affinity of this antibody to an Aβ(1-42) globulomer.

Further, the present invention relates to an antibody having a binding affinity to an Aβ(20-42) globulomer that is greater than the binding affinity of this antibody to an Aβ(12-42) globulomer.

According to a particular embodiment, the invention thus relates to antibodies having a binding affinity to the Aβ(20-42) globulomer that is greater than the binding affinity of the antibody to both the Aβ(1-42) globulomer and the Aβ(12-42) globulomer.

The term “Aβ(X-Y)” here refers to the amino acid sequence from amino acid position X to amino acid position Y of the human amyloid β protein including both X and Y, in particular to the amino acid sequence from amino acid position X to amino acid position Y of the amino acid sequence DAEFRHDSGY EVHHQKLVFF AEDVGSNKGA IIGLMVGGVV IAT (SEQ ID NO.:64) (corresponding to amino acid positions 1 to 43) or any of its naturally occurring variants, in particular those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide, including both position X and position Y or a sequence with up to three additional amino acid substitutions none of which may prevent globulomer formation, preferably with no additional amino acid substitutions in the portion from amino acid 12 or X, whichever number is higher, to amino acid 42 or Y, whichever number is lower, more preferably with no additional amino acid substitutions in the portion from amino acid 20 or X, whichever number is higher, to amino acid 42 or Y, whichever number is lower, and most preferably with no additional amino acid substitutions in the portion from amino acid 20 or X, whichever number is higher, to amino acid 40 or Y, whichever number is lower, an “additional” amino acid substation herein being any deviation from the canonical sequence that is not found in nature.

More specifically, the term “Aβ(1-42)” here refers to the amino acid sequence from amino acid position 1 to amino acid position 42 of the human amyloid β protein including both 1 and 42, in particular to the amino acid sequence DAEFRHDSGY EVHHQKLVFF AEDVGSNKGA IIGLMVGGVV IA (SEQ ID NO.:46) or any of its naturally occurring variants, in particular those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide, including both 1 and 42 or a sequence with up to three additional amino acid substitutions none of which may prevent globulomer formation, preferably with no additional amino acid substitutions in the portion from amino acid 20 to amino acid 42. Likewise, the term “Aβ(1-40)” here refers to the amino acid sequence from amino acid position 1 to amino acid position 40 of the human amyloid β protein including both 1 and 40, in particular to the amino acid sequence DAEFRHDSGY EVHHQKLVFF AEDVGSNKGA IIGLMVGGVV (SEQ ID NO.:47) or any of its naturally occurring variants, in particular those with at least one mutation selected from the group consisting of A2T, H6R, D7N, A21G (“Flemish”), E22G (“Arctic”), E22Q (“Dutch”), E22K (“Italian”), and D23N (“Iowa”) wherein the numbers are relative to the start of the Aβ peptide, including both 1 and 40 or a sequence with up to three additional amino acid substitutions none of which may prevent globulomer formation, preferably with no additional amino acid substitutions in the portion from amino acid 20 to amino acid 40.

More specifically, the term “Aβ(12-42)” here refers to the amino acid sequence from amino acid position 12 to amino acid position 42 of the human amyloid β protein including both 12 and 42, in particular to the amino acid sequence VHHQKLVFF AEDVGSNKGA IIGLMVGGVV IA (SEQ ID NO: 66) or any of its naturally occurring variants, in particular those with at least one mutation selected from the group consisting of A21G (“Flemish”), E22G (“Arctic”), E22Q (“Dutch”), E22K (“Italian”), D23N (“Iowa”), A42T and A42V wherein the numbers are relative to the start of the Aβ peptide, including both 12 and 42 or a sequence with up to three additional amino acid substitutions none of which may prevent globulomer formation, preferably with no additional amino acid substitutions in the portion from amino acid 20 to amino acid 42.

More specifically, the term “Aβ(20-42)” herein refers to the amino acid sequence from amino acid position 20 to amino acid position 42 of the human amyloid β protein including both 20 and 42, in particular to the amino acid sequence F AEDVGSNKGA IIGLMVGGVV IA (SEQ ID NO: 67) or any of its naturally occurring variants, in particular those with at least one mutation selected from the group consisting of A21G (“Flemish”), E22G (“Arctic”), E22Q (“Dutch”), E22K (“Italian”), D23N (“Iowa”), A42T and A42V wherein the numbers are relative to the start of the Aβ peptide, including both 20 and 42 or a sequence with up to three additional amino acid substitutions none of which may prevent globulomer formation, preferably without any additional amino acid substitutions.

The term “Aβ(X-Y) globulomer” (Aβ(X-Y) globular oligomer) here refers to a soluble, globular, non-covalent association of Aβ(X-Y) peptides as defined above, possessing homogeneity and distinct physical characteristics. According to one aspect, Aβ(X-Y) globulomers are stable, non-fibrillar, oligomeric assemblies of Aβ(X-Y) peptides which are obtainable by incubation with anionic detergents. In contrast to monomer and fibrils, these globulomers are characterized by defined assembly numbers of subunits (e.g. early assembly forms, n=4-6, “oligomers A”, and late assembly forms, n=12-14, “oligomers B”, as described in International Appln. Publication No. WO2004/067561). The globulomers have a 3-dimensional globular type structure (“molten globule”, see Barghorn et al., 2005, J Neurochem, 95, 834-847). They may be further characterized by one or more of the following features:

cleavability of N-terminal amino acids X-23 with promiscuous proteases (such as thermolysin or endoproteinase GluC) yielding truncated forms of globulomers;

non-accessibility of C-terminal amino acids 24-Y with promiscuous proteases and antibodies;

truncated forms of these globulomers maintain the 3-dimensional core structure of said globulomers with a better accessibility of the core epitope Aβ(20-Y) in its globulomer conformation.

According to the invention and in particular for the purpose of assessing the binding affinities of the antibodies of the present invention, the term “Aβ(X-Y) globulomer” here refers in particular to a product which is obtainable by a process as described in International Application Publication No. WO 2004/067561, which is incorporated herein by reference. Said process comprises unfolding a natural, recombinant or synthetic Aβ(X-Y) peptide or a derivative thereof; exposing the at least partially unfolded Aβ(X-Y) peptide or derivative thereof to a detergent, reducing the detergent action and continuing incubation.

For the purpose of unfolding the peptide, hydrogen bond-breaking agents such as, for example, hexafluoroisopropanol (HFIP) may be allowed to act on the protein. Times of action of a few minutes, for example about 10 to 60 minutes, are sufficient when the temperature of action is from about 20 to 50° C. and in particular about 35 to 40° C. Subsequent dissolution of the residue evaporated to dryness, preferably in concentrated form, in suitable organic solvents miscible with aqueous buffers, such as, for example, dimethyl sulfoxide (DMSO), results in a suspension of the at least partially unfolded peptide or derivative thereof, which can be used subsequently. If required, the stock suspension may be stored at low temperature, for example at about −20° C., for an interim period.

Alternatively, the peptide or the derivative thereof may be taken up in slightly acidic, preferably aqueous, solution, for example, an about 10 mM aqueous HCl solution. After an incubation time of usually a few minutes, insoluble components are removed by centrifugation. A few minutes at 10000 g is expedient. These method steps are preferably carried out at room temperature, i.e. a temperature in the range from 20 to 30° C. The supernatant obtained after centrifugation contains the Aβ(X-Y) peptide or the derivative thereof and may be stored at low temperature, for example at about −20° C., for an interim period.

The following exposure to a detergent relates to the oligomerization of the peptide or the derivative thereof to give an intermediate type of oligomers (in WO 2004/067561 referred to as oligomers A). For this purpose, a detergent is allowed to act on the at least partially unfolded peptide or derivative thereof until sufficient intermediate oligomer has been produced. Preference is given to using ionic detergents, in particular anionic detergents.

According to a particular embodiment, a detergent of the formula (I):

R—X,

is used, in which the radical R is unbranched or branched alkyl having from 6 to 20 and preferably 10 to 14 carbon atoms or unbranched or branched alkenyl having from 6 to 20 and preferably 10 to 14 carbon atoms, the radical X is an acidic group or salt thereof, with X being preferably selected from among —COO-M⁺, —SO₃-M⁺, and especially —OSO₃-M⁺ and M⁺ is a hydrogen cation or an inorganic or organic cation preferably selected from alkali metal and alkaline earth metal cations and ammonium cations. Advantageous are detergents of the formula (I), in which R is unbranched alkyl of which alk-1-yl radicals must be mentioned in particular. Particular preference is given to sodium dodecyl sulfate (SDS). Lauric acid and oleic acid can also be used advantageously. The sodium salt of the detergent lauroylsarcosin (also known as sarkosyl NL-30 or Gardol®) is also particularly advantageous. The time of detergent action in particular depends on whether (and if yes, to what extent) the peptide or the derivative thereof subjected to oligomerization has unfolded. If, according to the unfolding step, the peptide or derivative thereof has been treated beforehand with a hydrogen bond-breaking agent, i.e. in particular with hexafluoroisopropanol, times of action in the range of a few hours, advantageously from about 1 to 20 and in particular from about 2 to 10 hours, are sufficient when the temperature of action is about 20 to 50° C. and in particular about 35 to 40° C. If a less unfolded or an essentially not unfolded peptide or derivative thereof is the starting point, correspondingly longer times of action are expedient. If the peptide or the derivative thereof has been pretreated, for example, according to the procedure indicated above as an alternative to the HFIP treatment or said peptide or derivative thereof is directly subjected to oligomerization, times of action in the range from about 5 to 30 hours and in particular from about 10 to 20 hours are sufficient when the temperature of action is about 20 to 50° C. and in particular about 35 to 40° C. After incubation, insoluble components are advantageously removed by centrifugation. A few minutes at 10000 g is expedient.

The detergent concentration to be chosen depends on the detergent used. If SDS is used, a concentration in the range from 0.01 to 1% by weight, preferably from 0.05 to 0.5% by weight, for example of about 0.2% by weight, proves expedient. If lauric acid or oleic acid are used, somewhat higher concentrations are expedient, for example in a range from 0.05 to 2% by weight, preferably from 0.1 to 0.5% by weight, for example of about 0.5% by weight.

The detergent action should take place at a salt concentration approximately in the physiological range. Thus, in particular NaCl concentrations in the range from 50 to 500 mM, preferably from 100 to 200 mM and particularly at about 140 mM are expedient. The subsequent reduction of the detergent action and continuation of incubation relates to a further oligomerization to give the Aβ(X-Y) globulomer of the invention (in Internation Appln. Publication No. WO 2004/067561 referred to as oligomers B). Since the composition obtained from the preceding step regularly contains detergent and a salt concentration in the physiological range it is then expedient to reduce detergent action and, preferably, also the salt concentration. This may be carried out by reducing the concentration of detergent and salt, for example, by diluting, expediently with water or a buffer of lower salt concentration, for example Tris-HCl, pH 7.3. Dilution factors in the range from about 2 to 10, advantageously in the range from about 3 to 8 and in particular of about 4, have proved suitable. The reduction in detergent action may also be achieved by adding substances which can neutralize said detergent action. Examples of these include substances capable of complexing the detergents, like substances capable of stabilizing cells in the course of purification and extraction measures, for example particular EO/PO block copolymers, in particular the block copolymer under the trade name Pluronic® F 68. Alkoxylated and, in particular, ethoxylated alkyl phenols such as the ethoxylated t-octylphenols of the Triton® X series, in particular Triton® X100, 3-(3-cholamidopropyldimethylammonio)-1-propanesulfonate (CHAPS®) or alkoxylated and, in particular, ethoxylated sorbitan fatty esters such as those of the Tween® series, in particular Tween® 20, in concentration ranges around or above the particular critical micelle concentration, may be equally used. Subsequently, the solution is incubated until sufficient Aβ(X-Y) globulomer of the invention has been produced. Times of action in the range of several hours, preferably in the range from about 10 to 30 hours and in particular in the range from about 15 to 25 hours, are sufficient when the temperature of action is about 20 to 50° C. and in particular about 35 to 40° C. The solution may then be concentrated and possible residues may be removed by centrifugation. Here too, a few minutes at 10000 g proves expedient. The supernatant obtained after centrifugation contains an Aβ(X-Y) globulomer of the invention.

An Aβ(X-Y) globulomer of the invention can be finally recovered in a manner known per se, e.g. by ultrafiltration, dialysis, precipitation or centrifugation. It is further preferred if electrophoretic separation of the Aβ(X-Y) globulomers under denaturing conditions, e.g. by SDS-PAGE, produces a double band (e.g. with an apparent molecular weight of 38/48 kDa for Aβ(1-42)), and especially preferred if upon glutardialdehyde treatment of the globulomers before separation these two bands are merged into one. It is also preferred if size exclusion chromatography of the globulomers results in a single peak (e.g. corresponding to a molecular weight of approximately 100 kDa for Aβ(1-42) globulomer or of approximately 60 kDa for glutardialdehyde cross-linked Aβ(1-42) globulomer), respectively. Starting out from Aβ(1-42) peptide, Aβ(12-42) peptide, and Aβ(20-42) peptide said processes are in particular suitable for obtaining Aβ(1-42) globulomers, Aβ(12-42) globulomers, and Aβ(20-42) globulomers.

In a particular embodiment of the invention, Aβ(X-Y) globulomers wherein X is selected from the group consisting of the numbers 2 . . . 24 and Y is as defined above, are those which are obtainable by truncating Aβ(1-Y) globulomers into shorter forms wherein X is selected from the group consisting of the numbers 2 . . . 24, with X preferably being 20 or 12, and Y is as defined above, which can be achieved by treatment with appropriate proteases. For instance, an Aβ(20-42) globulomer can be obtained by subjecting an Aβ(1-42) globulomer to thermolysin proteolysis, and an Aβ(12-42) globulomer can be obtained by subjecting an Aβ(1-42) globulomer to endoproteinase GluC proteolysis. When the desired degree of proteolysis is reached, the protease is inactivated in a generally known manner. The resulting globulomers may then be isolated following the procedures already described herein and, if required, processed further by further work-up and purification steps. A detailed description of said processes is disclosed in International Appln. Publication No. WO 2004/067561, which is incorporated herein by reference.

For the purposes of the present invention, an Aβ(1-42) globulomer is, in particular, the Aβ(1-42) globulomer as described in Example Ib below; an Aβ(20-42) globulomer is in particular the Aβ(20-42) globulomer as described in Example 1a herein, and an Aβ(12-42) globulomer is in particular the Aβ(12-42) globulomer as described in Example 1c herein. Preferably, the globulomer shows affinity to neuronal cells. Preferably, the globulomer also exhibits neuromodulating effects. According to another aspect of the invention, the globulomer consists of 11 to 16, and most preferably, of 12 to 14 Aβ(X-Y) peptides.

According to another aspect of the invention, the term “Aβ(X-Y) globulomer” herein refers to a globulomer consisting essentially of Aβ(X-Y) subunits, where it is preferred if on average at least 11 of 12 subunits are of the Aβ(X-Y) type, more preferred if less than 10% of the globulomers comprise any non-Aβ(X-Y) peptides, and most preferred if the content of non-Aβ(X-Y) peptides is below the detection threshold. More specifically, the term “Aβ(1-42) globulomer” herein refers to a globulomer consisting essentially of Aβ(1-42) units as defined above; the term “Aβ(12-42) globulomer” herein refers to a globulomer consisting essentially of Aβ(12-42) units as defined above; and the term “Aβ(20-42) globulomer” herein refers to a globulomer consisting essentially of Aβ(20-42) units as defined above.

The term “cross-linked Aβ(X-Y) globulomer” herein refers to a molecule obtainable from an Aβ(X-Y) globulomer as described above by cross-linking, preferably chemically cross-linking, more preferably aldehyde cross-linking, most preferably glutardialdehyde cross-linking of the constituent units of the globulomer. In another aspect of the invention, a cross-linked globulomer is essentially a globulomer in which the units are at least partially joined by covalent bonds, rather than being held together by non-covalent interactions only. For the purposes of the present invention, a cross-linked Aβ(1-42) globulomer is in particular the cross-linked Aβ(1-42) oligomer as described in Example 1d herein.

The term “Aβ(X-Y) globulomer derivative” herein refers in particular to a globulomer that is labelled by being covalently linked to a group that facilitates detection, preferably a fluorophore, e.g. fluorescein isothiocyanate, phycoerythrin, Aequorea victoria fluorescent protein, Dictyosoma fluorescent protein or any combination or fluorescence-active derivative thereof; a chromophore; a chemoluminophore, e.g. luciferase, preferably Photinus pyralis luciferase, Vibrio fischeri luciferase, or any combination or chemoluminescence-active derivative thereof; an enzymatically active group, e.g. peroxidase, e.g. horseradish peroxidase, or any enzymatically active derivative thereof; an electron-dense group, e.g. a heavy metal containing group, e.g. a gold containing group; a hapten, e.g. a phenol derived hapten; a strongly antigenic structure, e.g. peptide sequence predicted to be antigenic, e.g. predicted to be antigenic by the algorithm of Kolaskar and Tongaonkar; an aptamer for another molecule; a chelating group, e.g. hexahistidinyl; a natural or nature-derived protein structure mediating further specific protein-protein interactions, e.g. a member of the fos/jun pair; a magnetic group, e.g. a ferromagnetic group; or a radioactive group, e.g. a group comprising ¹H, ¹⁴C, ³²P, ³⁵S or ¹²⁵I or any combination thereof; or to a globulomer flagged by being covalently or by non-covalent high-affinity interaction, preferably covalently linked to a group that facilitates inactivation, sequestration, degradation and/or precipitation, preferably flagged with a group that promotes in vivo degradation, more preferably with ubiquitin, where is particularly preferred if this flagged oligomer is assembled in vivo; or to a globulomer modified by any combination of the above. Such labelling and flagging groups and methods for attaching them to proteins are known in the art. Labelling and/or flagging may be performed before, during or after globulomerisation. In another aspect of the invention, a globulomer derivative is a molecule obtainable from a globulomer by a labelling and/or flagging reaction.

Correspondingly, term “Aβ(X-Y) monomer derivative” here refers in particular to an Aβ monomer that is labelled or flagged as described for the globulomer.

Expediently, the antibody of the present invention binds to an Aβ(20-42) globulomer with a K_{D} in the range of 1×10⁻⁶ M to 1×10⁻¹² M. Preferably, the antibody binds to an Aβ(20-42) globulomer with high affinity, for instance with a K_{D} of 1×10⁻⁷ M or greater affinity, e.g. with a K_{D} of 3×10⁻⁸ M or greater affinity, with a K_{D} of 1×10⁻⁸ M or greater affinity, e.g. with a K_{D} of 3×10⁻⁹ M or greater affinity, with a K_{D} of 1×10⁻⁹ M or greater affinity, e.g. with a K_{D} of 3×10⁻¹⁰ M or greater affinity, with a K_{D} of 1×10⁻¹⁰ M or greater affinity, e.g. with a K_{D} of 3×10⁻¹¹ M or greater affinity, or with a K_{D} of 1×10⁻¹¹ M or greater affinity.

The term “greater affinity” herein refers to a degree of interaction where the equilibrium between unbound antibody and unbound globulomer on the one hand and antibody-globulomer complex on the other is further in favour of the antibody-globulomer complex. Likewise, the term “smaller affinity” here refers to a degree of interaction where the equilibrium between unbound antibody and unbound globulomer on the one hand and antibody-globulomer complex on the other is further in favour of the unbound antibody and unbound globulomer. The term “greater affinity” is synonymous with the term “higher affinity” and term “smaller affinity” is synonymous with the term “lower affinity”.

According to a particular embodiment, the invention relates to an antibody which binds to the Aβ(20-42) globulomer with a K_{D} in the range of 1×10⁻⁶ M to 1×10⁻¹² M, to the Aβ(1-42) globulomer with a K_{D} of 10⁻¹² M or smaller affinity, the binding affinity to the Aβ(20-42) globulomer being greater than the binding affinity to the Aβ(1-42) globulomer.

It is preferred that the binding affinity of the antibody of the present invention to the Aβ(20-42) globulomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to the Aβ(1-42) globulomer.

According to a particular embodiment, the invention relates to an antibody which binds to the Aβ(12-42) globulomer with a K_{D} with a K_{D} of 10⁻¹² M or smaller affinity, the binding affinity to the Aβ(20-42) globulomer being greater than the binding affinity to the Aβ(12-42) globulomer.

It is also preferred that the binding affinity of the antibody of the present invention to the Aβ(20-42) globulomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to the Aβ(12-42) globulomer.

Preferably, the antibodies of the present invention bind to at least one Aβ globulomer, as defined above, and have a comparatively smaller affinity for at least one non-globulomer form of Aβ.

Antibodies of the present invention having a comparatively smaller affinity for at least one non-globulomer form of Aβ than for at least one Aβ globulomer include antibodies having a binding affinity to the Aβ(20-42) globulomer that is greater than to an Aβ(1-42) monomer. Further, it is preferred that, alternatively or additionally, the binding affinity of the antibody to the Aβ(20-42) globulomer is greater than to an Aβ(1-40) monomer.

In a preferred embodiment of the invention, the affinity of the antibody to the Aβ(20-42) globulomer is greater than its affinity to both the Aβ(1-40) and the Aβ(1-42) monomer.

The term “Aβ(X-Y) monomer” here refers to the isolated form of the Aβ(X-Y) peptide, preferably a form of the Aβ(X-Y) peptide which is not engaged in essentially non-covalent interactions with other Aβ peptides. Practically, the Aβ(X-Y) monomer is usually provided in the form of an aqueous solution. In a particularly preferred embodiment of the invention, the aqueous monomer solution contains 0.05% to 0.2%, more preferably about 0.1% NH₄OH. In another particularly preferred embodiment of the invention, the aqueous monomer solution contains 0.05% to 0.2%, more preferably about 0.1% NaOH. When used (for instance for determining the binding affinities of the antibodies of the present invention), it may be expedient to dilute said solution in an appropriate manner. Further, it is usually expedient to use said solution within 2 hours, in particular within 1 hour, and especially within 30 minutes after its preparation.

More specifically, the term “Aβ(1-40) monomer” here refers to an Aβ(1-40) monomer preparation as described herein, and the term “Aβ(1-42) monomer” here refers to an Aβ(1-42) preparation as described herein.

Expediently, the antibody of the present invention binds to one or, more preferably, both monomers with low affinity, most preferably with a K_{D} of 1×10⁻⁸ M or smaller affinity, e.g. with a K_{D} of 3×10⁻⁸ M or smaller affinity, with a K_{D} of 1×10⁻⁷ M or smaller affinity, e.g. with a K_{D} of 3×10⁻⁷ M or smaller affinity, or with a K_{D} of 1×10⁻⁶ M or smaller affinity, e.g. with a K_{D} of 3×10⁻⁵ M or smaller affinity, or with a K_{D} of 1×10⁻⁵ M or smaller affinity.

It is especially preferred that the binding affinity of the antibody of the present invention to the Aβ(20-42) globulomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to one or, more preferably, both monomers.

Antibodies of the present invention having a comparatively smaller affinity for at least one non-globulomer form of Aβ than for at least one Aβ globulomer further include antibodies having a binding affinity to the Aβ(20-42) globulomer that is greater than to Aβ(1-42) fibrils. Further, it is preferred that, alternatively or additionally, the binding affinity of the antibody to the Aβ(20-42) globulomer is greater than to Aβ(1-40) fibrils. The term “fibril” herein refers to a molecular structure that comprises assemblies of non-covalently associated, individual Aβ(X-Y) peptides, which show fibrillary structure in the electron microscope, which bind Congo red and then exhibit birefringence under polarized light and whose X-ray diffraction pattern is a cross-β structure.

In another aspect of the invention, a fibril is a molecular structure obtainable by a process that comprises the self-induced polymeric aggregation of a suitable Aβ peptide in the absence of detergents, e.g. in 0.1 M HCl, leading to the formation of aggregates of more than 24, preferably more than 100 units. This process is well known in the art. Expediently, Aβ(X-Y) fibrils are used in the form of an aqueous solution. In a particularly preferred embodiment of the invention, the aqueous fibril solution is made by dissolving the Aβ peptide in 0.1% NH₄OH, diluting it 1:4 with 20 mM NaH₂PO₄, 140 mM NaCl, pH 7.4, followed by readjusting the pH to 7.4, incubating the solution at 37° C. for 20 h, followed by centrifugation at 10000 g for 10 min and resuspension in 20 mM NaH₂PO₄, 140 mM NaCl, pH 7.4.

The term “Aβ(X-Y) fibril” herein also refers to a fibril comprising Aβ(X-Y) subunits where it is preferred if, on average, at least 90% of the subunits are of the Aβ(X-Y) type, more preferred, if at least 98% of the subunits are of the Aβ(X-Y) type and, most preferred, if the content of non-Aβ(X-Y) peptides is below the detection threshold. More specifically, the term “Aβ(1-42) fibril” herein refers to a Aβ(1-42) fibril preparation as described in Example IV.2.8.

Expediently, the antibody of the present invention binds to one or, more preferably, both fibrils with low affinity, most preferably with a K_{D} of 1×10⁻⁸ M or smaller affinity,

e.g. with a K_{D} of 3×10⁻⁸ M or smaller affinity, with a K_{D} of 1×10⁻⁷ M or smaller affinity, e.g. with a K_{D} of 3×10⁻⁷ M or smaller affinity, or with a K_{D} of 1×10⁻⁶ M or smaller affinity, e.g. with a K_{D} of 3×10⁻⁵ M or smaller affinity, or with a K_{D} of 1×10⁻⁵ M or smaller affinity.

It is especially preferred that the binding affinity of the antibody of the present invention to Aβ(20-42) globulomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to one or, more preferably, both fibrils.

According to one particular embodiment, the invention relates to antibodies having a binding affinity to the Aβ(20-42) globulomer which is greater than its binding affinity to both Aβ(1-40) and Aβ(1-42) fibrils.

According to a particularly preferred embodiment, the present invention relates to antibodies having a comparatively smaller affinity for both the monomeric and fibrillary forms of Aβ than for at least one Aβ globulomer, in particular Aβ(20-42) globulomer. These antibodies hereinafter are referred to globulomer-specific antibodies.

It is noted that the antibodies of the present invention may also be reactive with, i.e. bind to, Aβ forms other than the Aβ globulomers described herein. These antigens may or may not be oligomeric or globulomeric. Thus, the antigens to which the antibodies of the present invention bind include any Aβ form that comprises the globulomer epitope with which the antibodies of the present invention are reactive. Such Aβ forms include truncated and non-truncated Aβ(X-Y) forms (with X and Y being defined as above), such as Aβ(20-42), Aβ(20-40), Aβ(12-42), Aβ(12-40), Aβ(1-42), and Aβ(1-40) forms, provided that said forms comprise the globulomer epitope.

Turning back to humanized antibodies 7C6 and 5F7, these Aβ(20-42) globulomer-specific antibodies recognize predominantly Aβ(20-42) globulomer forms and not standard preparations of Aβ(1-40) monomers, Aβ(1-42) monomers, Aβ-fibrils or sAPP (i.e, Aβ precursor) in contrast to, for example, competitor antibodies such as m266 and 3D6. Such specificity for globulomers is important because specifically targeting the globulomer form of Aβ with a globulomer preferential antibody such as, for example, humanized 7C6 or humanized 5F7 will: 1) avoid targeting insoluble amyloid deposits, binding to which may account for inflammatory side effects observed during immunizations with insoluble Aβ; 2) spare Aβ monomer and APP that are reported to have precognitive physiological functions (Plan et al., J. of Neuroscience 23:5531-5535 (2003); and 3) increase the bioavailability of the antibody, as it would not be shaded or inaccessible through extensive binding to insoluble deposits.

The subject invention also includes isolated nucleotide sequences (and fragments thereof) encoding the variable light and heavy chains of humanized antibody 7C6 or humanized 5F7 as well as those nucleotide sequences (or fragments thereof) having sequences comprising, corresponding to, identical to, hybridizable to, or complementary to, at least about 70% (e.g., 70% 71%, 72%, 73%, 74%, 75%, 76%, 77%, 78% or 79%), preferably at least about 80% (e.g., 80%, 81%, 82%, 83%, 84%, 85%, 86%, 87%, 88% or 89%), and more preferably at least about 90% (e.g, 91%, 92%, 93%, 94%, 95%, 96%, 97%, 98%, 99% or 100%) identity to these encoding nucleotide sequences. (All integers (and portions thereof) between and including 70% and 100% are considered to be within the scope of the present invention with respect to percent identity.) Such sequences may be derived from any source (e.g., either isolated from a natural source, produced via a semi-synthetic route, or synthesized de novo). In particular, such sequences may be isolated or derived from sources other than described in the examples (e.g., bacteria, fungus, algae, mouse or human).

In addition to the nucleotide sequences described above, the present invention also includes amino acid sequences of the variable light and heavy chains of humanized antibody 7C6 and humanized antibody 5F7 (or fragments of these amino acid sequences). Further, the present invention also includes amino acid sequences (or fragments thereof) comprising, corresponding to, identical to, or complementary to at least about 70% (e.g., 70%, 71%, 72%, 73%, 74%, 75%, 76%, 77%, 78% or 79%), preferably at least about 80% (e.g., 80% 81%, 82%, 83%, 84%, 85%, 86%, 87%, 88% or 89%), and more preferably at least about 90% identity (e.g., 90%, 91%, 92%, 93%, 94%, 95%, 96%, 97%, 98%, 99% or 100%), to the amino acid sequences of the proteins of the present invention. (Again, all integers (and portions thereof) between and including 70% and 100% (as recited in connection with the nucleotide sequence identities noted above) are also considered to be within the scope of the present invention with respect to percent identity.)

For purposes of the present invention, a “fragment” of a nucleotide sequence is defined as a contiguous sequence of approximately at least 6, preferably at least about 8, more preferably at least about 10 nucleotides, and even more preferably at least about 15 nucleotides corresponding to a region of the specified nucleotide sequence.

The term “identity” refers to the relatedness of two sequences on a nucleotide-by-nucleotide basis over a particular comparison window or segment. Thus, identity is defined as the degree of sameness, correspondence or equivalence between the same strands (either sense or antisense) of two DNA segments (or two amino acid sequences). “Percentage of sequence identity” is calculated by comparing two optimally aligned sequences over a particular region, determining the number of positions at which the identical base or amino acid occurs in both sequences in order to yield the number of matched positions, dividing the number of such positions by the total number of positions in the segment being compared and multiplying the result by 100. Optimal alignment of sequences may be conducted by the algorithm of Smith & Waterman, Appl. Math. 2:482 (1981), by the algorithm of Needleman & Wunsch, J. Mol. Biol. 48:443 (1970), by the method of Pearson & Lipman, Proc. Natl. Acad. Sci. (USA) 85:2444 (1988) and by computer programs which implement the relevant algorithms (e.g., Clustal Macaw Pileup (http://cmgm.stanford.edu/biochem218/11Multiple.pdf; Higgins et al., CABIOS. 5L151-153 (1989)), FASTDB (Intelligenetics), BLAST (National Center for Biomedical Information; Altschul et al., Nucleic Acids Research 25:3389-3402 (1997)), PILEUP (Genetics Computer Group, Madison, Wis.) or GAP, BESTFIT, FASTA and TFASTA (Wisconsin Genetics Software Package Release 7.0, Genetics Computer Group, Madison, Wis.). (See U.S. Pat. No. 5,912,120.)

For purposes of the present invention, “complementarity” is defined as the degree of relatedness between two DNA segments. It is determined by measuring the ability of the sense strand of one DNA segment to hybridize with the anti-sense strand of the other DNA segment, under appropriate conditions, to form a double helix. A “complement” is defined as a sequence which pairs to a given sequence based upon the canonic base-pairing rules. For example, a sequence A-G-T in one nucleotide strand is “complementary” to T-C-A in the other strand.

In the double helix, adenine appears in one strand, thymine appears in the other strand. Similarly, wherever guanine is found in one strand, cytosine is found in the other. The greater the relatedness between the nucleotide sequences of two DNA segments, the greater the ability to form hybrid duplexes between the strands of the two DNA segments.

“Similarity” between two amino acid sequences is defined as the presence of a series of identical as well as conserved amino acid residues in both sequences. The higher the degree of similarity between two amino acid sequences, the higher the correspondence, sameness or equivalence of the two sequences. (“Identity between two amino acid sequences is defined as the presence of a series of exactly alike or invariant amino acid residues in both sequences.) The definitions of “complementarity”, “identity” and “similarity” are well known to those of ordinary skill in the art.

“Encoded by” refers to a nucleic acid sequence which codes for a polypeptide sequence, wherein the polypeptide sequence or a portion thereof contains an amino acid sequence of at least 3 amino acids, more preferably at least 8 amino acids, and even more preferably at least 15 amino acids from a polypeptide encoded by the nucleic acid sequence.

“Biological activity” as used herein, refers to all inherent biological properties of the Aβ(20-42) region of the globulomer. Such properties include, for example, the ability to bind to the humanized 7C6 or humanized 5F7 antibodies described herein.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering” as used herein, refers to the process of rendering a chemical species such as a polypeptide substantially free of naturally associated components by isolation, e.g., using protein purification techniques well known in the art.

The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG3, IgG4, IgA1 and IgA2) or subclass.

The term “antigen-binding portion” of an antibody (or simply “antibody portion”), as used herein, refers to one or more fragments of an antibody that retain the ability to specifically bind to an antigen (e.g., Aβ(20-42) globulomer). It has been shown that the antigen-binding function of an antibody can be performed by one or more fragments of a full-length antibody. Such antibody embodiments may also be bispecific, dual specific, or multispecific, specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., International Appln. Publication No. WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more the antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art and represented in Table 2.

TABLE 2

SEQUENCE OF HUMAN IgG HEAVY CHAIN CONSTANT
DOMAIN AND LIGHT CHAIN CONSTANT DOMAIN

| | Sequence | 123456789012345678901234567 |
|---|---|---|
| Protein | Identifier | 89012 |
| Ig gamma-1 | SEQ ID | ASTKGPSVFFLAPSSKSTSGGTAALGC |
| constant | NO.:38 | LVKDYFPEPVTVSWNSGALTSGVHTFP |
| region | | AVLQSSGLYSLSSVVTVPSSSLGTQTY |
| | | ICNVNHKPSNTKVDKKVEPKSCDKTHT |
| | | CPPCPAPELLGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPGK |
| Ig gamma-1 | SEQ ID | ASTKGPSVFPLAPSSKSTSGGTAALGC |
| constant | NO.:39 | LVKDYFPEPVTVSWNSGALTSGVHTFP |
| region | | AVLQSSGLYSLSSVVTVPSSSLGTQTY |
| mutant | | ICNVNHKPSNTKVDKKVEPKSCDKTHT |
| | | CPPCPAPEAAGGPSVFLFPPKPKDTLM |
| | | ISRTPEVTCVVVDVSHEDPEVKFNWYV |
| | | DGVEVHNAKTKPREEQYNSTYRVVSVL |
| | | TVLHQDWLNGKEYKCKVSNKALPAPIE |
| | | KTISKAKGQPREPQVYTLPPSREEMTK |
| | | NQVSLTCLVKGFYPSDIAVEWESNGQP |
| | | ENNYKTTPPVLDSDGSFFLYSKLTVDK |
| | | SRWQQGNVFSCSVMHEALHNHYTQKSL |
| | | SLSPGK |
| Ig Kappa | SEQ ID | TVAAPSVFIFPPSDEQLKSGTASVVCL |
| constant | NO.:40 | LNNFYPREAKVQWKVDNALQSGNSQES |
| region | | VTEQDSKDSTYSLSSTLTLSKADYEKH |
| | | KVYACEVTHQGLSSPVTKSFNRGEC |
| Ig Lambda | SEQ ID | QPKAAPSVTLFPPSSEELQANKATLVC |
| constant | NO.:41 | LISDFYPGAVTVAWKADSSPVKAGVET |
| region | | TTPSKQSNNKYAASSYLSLTPEQWKSH |
| | | RSYSCQVTHEGSTVEKTVAPTECS |

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds Aβ(20-42) globulomer and/or any other Aβ form that comprises the globulomer epitope with which the antibodies of the present invention are reactive and is substantially free of antibodies that specifically bind antigens other than Aβ(20-42) globulomer and/or any other Aβ form that comprises the globulomer epitope with which the antibodies of the present invention are reactive). An isolated antibody that specifically binds Aβ(20-42) globulomer may, however, have cross-reactivity to other antigens, such as Aβ(20-42) globulomer molecules from other species. Moreover, an isolated antibody may be substantially free of other cellular material and/or chemicals and/or any other Aβ form that comprises the globulomer epitope with which the antibodies of the present invention are reactive.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described below), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In a preferred embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art. In one embodiment of the invention the human heavy chain and light chain acceptor sequences are selected from the sequences described below:

TABLE 3
HEAVY CHAIN ACCEPTOR SEQUENCES

| | | |
|---|---|---|
| SEQ | | |
| ID | Protein | |
| No. | region | Sequence |

| | | |
|---|---|---|
| 48 | VH1-46/JH4 Fr1 | QVQLVQSGAEVKKPGASVKVSCKASGYTFT |
| 49 | VH1-46/JH4 Fr2 | WVRQAPGQGLEWMG |
| 50 | VH1-46/JH4 Fr3 | RVTMTRDTSTSTVYMELSSLRSEDTAVYYCAR |
| 51 | VH1-46/JH4 Fr4 | WGQGTLVTVSS |
| 52 | VH3-21/JH4 Fr1 | EVQLVESGGGLVKPGGSLRLSCAASGFTFS |
| 53 | VH3-21/JH4 Fr2 | WVRQAPGKGLEWVS |
| 54 | VH3-21/JH4 Fr3 | RFTISRDNAKNSLYLQMNSLRAEDTAVYYCAR |
| 55 | VH3-21/JH4 Fr4 | WGQGTLVTVSS |

TABLE 4
LIGHT CHAIN ACCEPTOR SEQUENCES

| SEQ | | |
|---|---|---|
| ID | Protein | |
| No. | region | Sequence |
| 56 | A19/JK1 Fr1 | DIVMTQSPLSLPVTPGEPASISC |
| 57 | A19/JK1 Fr2 | WYLQKPGQSPQLLIY |
| 58 | A19/JK1 Fr3 | GVPDRFSSGSGTDFTLKISRVEAEDVGVYYC |
| 59 | A19/JK1 Fr4 | FGGGTKVEIKR |
| 60 | A19/JK2 Fr1 | DIVMTQSPLSLPVTPGEPASISC |
| 61 | A19/JK2 Fr2 | WYLQKPGQSPQLLIY |
| 62 | A19/JK2 Fr3 | GVPDRFSGSGSGTDFTLKILSRVEAEDVGVYYC |
| 63 | A19/JK2 Fr4 | FGQGTKLEIKR |

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): 183-200 (2002); Marchalonis et al., Adv Exp Med. Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, more preferably at least 90%, more preferably at least 95%, more preferably at least 98% and most preferably at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

As used herein, the term “neutralizing” refers to neutralization of biological activity of a globulomer when a binding protein specifically binds the globulomer. Preferably, a neutralizing binding protein is a neutralizing antibody whose binding to the Aβ(20-42) amino acid region of the globulomer and/or any other Aβ form that comprises the globulomer epitope with which the antibodies of the present invention are reactive, results in inhibition of a biological activity of the globulomer. Preferably, the neutralizing binding protein binds to the Aβ(20-42) region of the globulomer and/or any other Aβ form that comprises the globulomer epitope with which the antibodies of the present invention are reactive, and reduces a biologically activity of the globulomer by at least about 20%, 40%, 60%, 80%, 85% or more. Inhibition of a biological activity of the globulomer by a neutralizing binding protein can be assessed by measuring one or more indicators of globulomer biological activity well known in the art.

The term “activity” includes activities such as the binding specificity/affinity of an antibody for an antigen, for example, an anti-Aβ(20-42) antibody or antibody to any other Aβ form that comprises the globulomer epitope with which the antibodies of the present invention are reactive, that binds to an Aβ(20-42) globulomer (and/or any other Aβ form that comprises the globulomer epitope with which the antibodies of the present invention are reactive) and/or the neutralizing potency of an antibody, for example, an anti-Aβ(20-42) antibody whose binding to Aβ(20-42) inhibits the biological activity of the globulomer and/or any other Aβ form that comprises the globulomer epitope with which the antibodies of the present invention are reactive.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “Kₒₙ”, as used herein, is intended to refer to the on rate constant for association of an antibody to the antigen to form the antibody/antigen complex as is known in the art.

The term “K_{off}”, as used herein, is intended to refer to the off rate constant for dissociation of an antibody from the antibody/antigen complex as is known in the art.

The term “K_{d}”, as used herein, is intended to refer to the dissociation constant of a particular antibody-antigen interaction as is known in the art.

The term “labeled binding protein” as used herein, refers to a protein with a label incorporated that provides for the identification of the binding protein. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. Preferably the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The terms “crystal”, and “crystallized” as used herein, refer to an antibody, or antigen-binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ed., pp. 20 1-16, Oxford University Press, New York, N.Y., (1999).

The term “polynucleotide” as referred to herein means a polymeric form of two or more nucleotides, either ribonucleotides or deoxynucleotides or a modified form of either type of nucleotide. The term includes single and double stranded forms of DNA but preferably is double-stranded DNA.

The term “isolated polynucleotide” as used herein shall mean a polynucleotide (e.g., of genomic, cDNA, or synthetic origin, or some combination thereof) that, by virtue of its origin, is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells that transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Preferably, host cells include prokaryotic and eukaryotic cells selected from any of the Kingdoms of life. Preferred eukaryotic cells include protist, fungal, plant and animal cells. Most preferably, host cells include but are not limited to the prokaryotic cell line E. coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

“Transgenic organism”, as known in the art and as used herein, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of Aβ(20-42) globulomer). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of Aβ(20-42) globulomer). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in International Application Publication No. WO 01/83525.

The term “agonist”, as used herein, refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, Aβ(20-42) globulomer polypeptides or polypeptides, nucleic acids, carbohydrates, or any other molecules that bind to Aβ(20-42) globulomer.

The term “antagonist” or “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological activity of Aβ(20-42) globulomer and/or any other Aβ form that comprises the globulomer epitope with which the antibodies of the present invention are reactive. Antagonists and inhibitors of Aβ(20-42) globulomer may include, but are not limited to, proteins, nucleic acids, carbohydrates, or any other molecules, which bind to Aβ(20-42) globulomer and/or any other Aβ form that comprises the globulomer epitope with which the antibodies of the present invention are reactive.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other mammalian or non-mammalian animals. Such substances include, but are not limited to, blood, serum, urine, synovial fluid, cells, organs, tissues (e.g., brain), bone marrow, lymph nodes, cerebrospinal fluid, and spleen.

I. Antibodies that Bind Aβ(20-42) Globulomer

One aspect of the present invention provides isolated murine monoclonal antibodies, or antigen-binding portions thereof, that bind to Aβ(20-42) globulomer and/or any other Aβ form that comprises the globulomer epitope with which the antibodies of the present invention are reactive, with high affinity, a slow off rate and high neutralizing capacity. A second aspect of the invention provides chimeric antibodies that bind Aβ(20-42) globulomer and/or any other Aβ form that comprises the globulomer epitope with which the antibodies of the present invention are reactive. A third aspect of the invention provides CDR grafted antibodies, or antigen-binding portions thereof, that bind Aβ(20-42) globulomer and/or any other Aβ form that comprises the globulomer epitope with which the antibodies of the present invention are reactive. A fourth aspect of the invention provides humanized antibodies, or antigen-binding portions thereof, that bind Aβ(20-42) globulomer and/or any other Aβ form that comprises the globulomer epitope with which the antibodies of the present invention are reactive. Preferably, the antibodies, or portions thereof, are isolated antibodies. Preferably, the antibodies of the invention neutralize Aβ(20-42) globulomer and/or any other Aβ form that comprises the globulomer epitope with which the antibodies of the present invention are reactive.

### Method of Making Anti-Aβ(20-42) Globulomer Antibodies

Antibodies of the present invention may be made by any of a number of techniques known in the art.

### 1. Anti-Aβ(20-42) Globulomer Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In one embodiment, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. Briefly, mice can be immunized with an Aβ(20-42) globulomer antigen. In a preferred embodiment, the antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with an Aβ(20-42) globulomer antigen, antibodies and/or antibody-producing cells may be obtained from the animal. An anti-Aβ(20-42) globulomer antibody-containing serum is obtained from the animal by bleeding or sacrificing the animal. The serum may be used as it is obtained from the animal, an immunoglobulin fraction may be obtained from the serum, or the anti-Aβ(20-42) globulomer antibodies may be purified from the serum. Serum or immunoglobulins obtained in this manner are polyclonal, thus having a heterogeneous array of properties.

Once an immune response is detected, e.g., antibodies specific for the antigen Aβ(20-42) globulomer are detected in the mouse serum, the mouse spleen is harvested and splenocytes isolated. The splenocytes are then fused by well-known techniques to any suitable myeloma cells, for example cells from cell line SP20 available from the American Type Culture Collection (Manassas, Va.). Hybridomas are selected and cloned by limited dilution. The hybridoma clones are then assayed by methods known in the art for cells that secrete antibodies capable of binding Aβ(20-42) globulomer. Ascites fluid, which generally contains high levels of antibodies, can be generated by immunizing mice with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using Aβ(20-42) globulomer, or a portion thereof, or a cell expressing Aβ(20-42) globulomer. In a preferred embodiment, the initial screening is performed using an enzyme-linked immunoassay (ELISA) or a radioimmunoassay (RIA), preferably an ELISA. An example of ELISA screening is provided in International Application Publication No. WO 00/37504, herein incorporated by reference.

Anti-Aβ(20-42) globulomer antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the hybridomas are mouse hybridomas, as described above. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-Aβ(20-42) globulomer antibody.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

### 2. Anti-Aβ(20-42) Globulomer Monoclonal Antibodies Using SLAM

In another aspect of the invention, recombinant antibodies are generated from single, isolated lymphocytes using a procedure referred to in the art as the selected lymphocyte antibody method (SLAM), as described in U.S. Pat. No. 5,627,052, International Application Publication No. WO 92/02551 and Babcock, J. S. et al. (1996) Proc. Natl. Acad. Sci. USA 93:7843-7848. In this method, single cells secreting antibodies of interest, e.g., lymphocytes derived from any one of the immunized animals described in Section 1, are screened using an antigen-specific hemolytic plaque assay, wherein the antigen Aβ(20-42) globulomer, a subunit of Aβ(20-42) globulomer, or a fragment thereof, is coupled to sheep red blood cells using a linker, such as biotin, and used to identify single cells that secrete antibodies with specificity for Aβ(20-42) globulomer. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to Aβ(20-42) globulomer. The amplified immunoglobulin sequences further can be manipulated in vitro, such as by in vitro affinity maturation methods such as those described in International Application Publication No. WO 97/29131 and International Application Publication No. WO 00/56772.

### 3. Anti-Aβ(20-42) Globulomer Monoclonal Antibodies Using Transgenic Animals

In another embodiment of the instant invention, antibodies are produced by immunizing a non-human animal comprising some, or all, of the human immunoglobulin locus with an Aβ(20-42) globulomer antigen. In a preferred embodiment, the non-human animal is a XENOMOUSE transgenic mouse, an engineered mouse strain that comprises large fragments of the human immunoglobulin loci and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also Internation Appln. Publication No. WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00/09560, published Feb. 24, 2000 and WO 00/037504, published Jun. 29, 2000. The XENOMOUSE transgenic mouse produces an adult-like human repertoire of fully human antibodies and generates antigen-specific human Mabs. The XENOMOUSE transgenic mouse contains approximately 80% of the human antibody repertoire through introduction of megabase sized, germline configuration YAC fragments of the human heavy chain loci and x light chain loci. See Mendez et al., Nature Genetics 15:146-156 (1997), Green and Jakobovits J. Exp. Med. 188:483-495 (1998), the disclosures of which are hereby incorporated by reference.

### 4. Anti-Aβ(20-42) Globulomer Monoclonal Antibodies Using Recombinant Antibody Libraries

In vitro methods also can be used to make the antibodies of the invention, wherein an antibody library is screened to identify an antibody having the desired binding specificity. Methods for such screening of recombinant antibody libraries are well known in the art and include methods described in, for example, Ladner et al., U.S. Pat. No. 5,223,409; Kang et al., International Appln. Publication No. WO 92/18619; Dower et al., International Appln. Publication No. WO 91/17271; Winter et al., International Appln. Publication No. WO 92/20791; Markland et al., International Appln. Publication No. WO 92/15679; Breitling et al., International Appln. Publication No. WO 93/01288; McCafferty et al., PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al., (1992) J Mol Biol 226:889-896; Clackson et al., (1991) Nature 352:624-628; Gram et al., (1992) PNAS 89:3576-3580; Garrad et al. (1991) Bio/Technology 9:1373-1377; Hoogenboom et al. (1991), Nuc Acid Res 19:4133-4137; and Barbas et al. (1991), PNAS 88:7978-7982, U.S. Patent Application Publication No. 20030186374, and International Application Publication No. WO 97/29131, the contents of each of which are incorporated herein by reference.

The recombinant antibody library may be from a subject immunized with Aβ(20-42) globulomer, or a portion of Aβ(20-42) globulomer. Alternatively, the recombinant antibody library may be from a naïve subject, i.e., one who has not been immunized with Aβ(20-42) globulomer, such as a human antibody library from a human subject who has not been immunized with human Aβ(20-42) globulomer. Antibodies of the invention are selected by screening the recombinant antibody library with the peptide comprising human Aβ(20-42) globulomer to thereby select those antibodies that recognize Aβ(20-42) globulomer and discriminate Aβ(1-42) globulomer, Aβ(1-40) and Aβ(1-42) monomer, Aβ-fibrils and sAPPα. Methods for conducting such screening and selection are well known in the art, such as described in the references in the preceding paragraph. To select antibodies of the invention having particular binding affinities for Aβ(20-42) globulomer and discriminate Aβ(1-42) globulomer, Aβ(1-40) and Aβ(1-42) monomer, Aβ-fibrils and sAPPα, such as those that dissociate from human Aβ(20-42) globulomer with a particular k_{off} rate constant, the art-known method of dot blot can be used to select antibodies having the desired k_{off} rate constant. To select antibodies of the invention having a particular neutralizing activity for Aβ(20-42) globulomer and discriminate Aβ(1-42) globulomer, Aβ(1-40) and Aβ(1-42) monomer, Aβ-fibrils and sAPPα, such as those with a particular an IC₅₀, standard methods known in the art for assessing the inhibition of human Aβ(20-42) globulomer activity may be used.

In one aspect, the invention pertains to an isolated antibody, or an antigen-binding portion thereof, that binds human Aβ(20-42) globulomer and discriminates Aβ(1-42) globulomer, Aβ(1-40) and Aβ(1-42) monomer, Aβ-fibrils and sAPPα. Preferably, the antibody is a neutralizing antibody. In various embodiments, the antibody is a recombinant antibody or a monoclonal antibody.

For example, the antibodies of the present invention can also be generated using various phage display methods known in the art. In phage display methods, functional antibody domains are displayed on the surface of phage particles that carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); International Application No. PCT/GB91/01134; International Appln. Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, each of which is incorporated herein by reference in its entirety.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in International Application Publ. No. WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Alternative to screening of recombinant antibody libraries by phage display, other methodologies known in the art for screening large combinatorial libraries can be applied to the identification of dual specificity antibodies of the invention. One type of alternative expression system is one in which the recombinant antibody library is expressed as RNA-protein fusions, as described in International Appln. Publication No. WO 98/31700 by Szostak and Roberts, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

In another approach the antibodies of the present invention can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present invention include those disclosed Wittrup, et al., U.S. Pat. No. 6,699,658 incorporated herein by reference.

### B. Production of Recombinant Aβ(20-42) Globulomer Antibodies

Antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although, it is possible to express the antibodies of the invention in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

### 1. Anti-Aβ(20-42) Globulomer Antibodies

Table 5 below includes a list of amino acid sequences of VH and VL regions of preferred anti-Aβ(20-42) globulomer humanized antibodies of the invention. The isolated anti-Aβ(20-42) globulomer antibody CDR sequences herein establish a novel family of Aβ(20-42) globulomer (and/or any other Aβ form that comprises the globulomer epitope with which the antibodies of the present invention are reactive) binding proteins, isolated in accordance with this invention, and comprising polypeptides that include the CDR sequences listed herein.

To generate and to select CDRs of the invention having preferred Aβ(20-42) globulomer binding and/or neutralizing activity with respect to Aβ(20-42) globulomer and/or any other Aβ form that comprises the globulomer epitope with which the antibodies of the present invention are reactive, standard methods known in the art for generating binding proteins of the present invention and assessing the Aβ(20-42) globulomer (and/or any other Aβ form that comprises the globulomer epitope with which the antibodies of the present invention are reactive) binding and/or neutralizing characteristics of those binding protein may be used, including but not limited to those specifically described herein.

### 2. Anti-Aβ(20-42) Globulomer Chimeric Antibodies

A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art and discussed in detail herein. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entireties. In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454 which are incorporated herein by reference in their entireties) by splicing genes from a mouse antibody molecule of appropriate antigen specificity together with genes from a human antibody molecule of appropriate biological activity can be used.

In one embodiment, the chimeric antibodies of the invention are produced by replacing the heavy chain constant region of the murine monoclonal anti-human Aβ(20-42) globulomer antibodies 5F7 and 7C6 described in International Appln. No. PCT/US2006/046148 filed on Nov. 30, 2006 with a human IgG1 constant region. In a specific embodiment, the chimeric antibody of the invention comprises the 5F7 heavy chain variable region (V_{H}) comprising the amino acid sequence of SEQ ID NOs.: 11, 12 and 13 and the 5F7 light chain variable region (V_{L}) comprising the amino acid sequence of SEQ ID NOs: 14, 15 and 15A. Alternatively, in another embodiment of the present invention, the chimeric antibody comprises the 7C6 heavy chain variable region (V_{H}) comprising the amino acid sequence of SEQ ID NOs.: 16, 17 and 18 and 7C6 light chain variable region (V_{L}) comprising the amino acid sequence of SEQ ID NOs.: 19, 20 and 21.

### 3. Anti-Aβ(20-42) Globulomer CDR Grafted Antibodies

CDR-grafted antibodies of the invention comprise heavy and light chain variable region sequences from a human antibody wherein one or more of the CDR regions of V_{H} and/or V_{L} are replaced with CDR sequences of the murine antibodies of the invention. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, it is preferable that the human variable framework that is chosen to replace the murine variable framework apart from the CDRs have at least a 65% sequence identity with the murine antibody variable region framework. It is more preferable that the human and murine variable regions apart from the CDRs have at least 70% sequence identify. It is even more preferable that the human and murine variable regions apart from the CDRs have at least 75% sequence identity. It is most preferable that the human and murine variable regions apart from the CDRs have at least 80% sequence identity. Methods for producing chimeric antibodies are known in the art and discussed in detail herein (See also EP 239,400; Internation Appln. Publication No. WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352).

### 4. Anti-Aβ(20-42) Globulomer Humanized Antibodies

Humanized antibodies are antibody molecules from non-human species antibody that bind the desired antigen having one or more complementarity determining regions (CDRs) from the non-human species and framework regions from a human immunoglobulin molecule.

Table 5 below illustrates the preferred humanized sequences of the present invention and the CDRs contained therein.

TABLE 5

List of Amino Acid Sequences of VH and VL regions of
humanized antibodies

| | | |
|---|---|---|
| SEQ | | Sequence |
| ID | Protein | 123456789012345678901234567 |
| No. | region | 890 |

| | | | |
|---|---|---|---|
| 1 | VH 5F7hum8 | | EVQLVQSGAEVKKPGASVKVSCKASG |
| | | | YTFTTFYIHWVRQAPGQGLEWIGMIGP |
| | | | GSGNTYYNEMFKDKATLTVDTSTSTAY |
| | | | MELSSLRSEDTAVYYCARAKSARAAW |
| | | | FAYWGQGTLVTVSS |
| | VH5F7hum8 | Residues 31- | TFYIH |
| | CDR-H1 | 35 of SEQ ID | (SEQ ID NO.:11) |
| | | NO.:1 | |
| | VH 5F7hum8 | Residues 50- | MIGPGSGNTYYNEMFKD |
| | CDR-H2 | 66 of SEQ ID | (SEQ ID NO.:12) |
| | | NO.:1 | |
| | VH 5F7hum8 | Residues 98- | AKSARAAWFAY |
| | CDR-H3 | 108 of SEQ ID | (SEQ ID NO.:13) |
| | | NO.:1 | |
| | VL 5F7hum8 | | DIVMTQSPLSLPVTPGEPASISCRSSQSV |
| | | | VQSNGNTYLEWYLQKPGQSPQLLIYKV |
| | | | SNRFSGVPDRFSGSGSGTDFTLKISRVE |
| | | | AEDVGVYYCFQGSHVPPTFGGGTKVEI |
| | | | KR |
| | VL 5F7hum8 | Residues 24- | RSSQSVVQSNGNTYLE |
| | CDR-L1 | 39 of SEQ ID | (SEQ ID NO.:14) |
| | | NO.:2 | |
| | VL 5F7hum8 | Residues 55- | KVSNRFS |
| | CDR-L2 | 61 of SEQ ID | (SEQ ID NO.:15) |
| | | NO.:2 | |
| | VL 5F7hum8 | Residues 94- | FQGSHVPPT |
| | CDR-L3 | 102 of SEQ ID | (SEQ ID NO.:65) |
| | | NO.:2 | |
| 3 | VH 7C6hum7 | | EVKLVESGGGLVKPGGSLRLSCAASGF |
| | | | TFSSYAMSWVRQAPGKGLEWVASIHN |
| | | | RGTIFYLDSVKGRFTISRDNVRNTLYLQ |
| | | | MNSLRAEDTAVYYCTRGRSNSYAMDY |
| | | | WGQGTSVTVSS |
| | VH 7C6hum7 | Residues 31- | SYAMS |
| | CDR-H1 | 35 of SEQ ID | (SEQ ID NO.:16) |
| | | NO.:3 | |
| | VH 7C6hum7 | Residues 50- | SIHNRGTIFYLDSVKG |
| | CDR-H2 | 65 of SEQ ID | (SEQ ID NO.:17) |
| | | NO.:3 | |
| | VH 7C6hum7 | Residues 98- | GRSNSYAMDY |
| | CDR-H3 | 107 of SEQ ID | (SEQ ID NO.:18) |
| | | NO.:3 | |
| 4 | VL 7C6hum7 | | DVLVTQSPLSLPVTPGEPASISCRSTQTL |
| | | | VHRNGDTYLEWYLQKPGQSPQSLIYKV |
| | | | SNRFSGVPDRFSGSGSGTDFTLKISRVE |
| | | | AEDVGVYYCFQGSHVPYTFGQGTKLEI |
| | | | KR |
| | VL 7C6hum7 | Residues 24- | RSTQTLVHRNGDTYLE |
| | CDR-L1 | 39 of SEQ ID | (SEQ ID NO.:19) |
| | | NO.:4 | |
| | VL 7C6hum7 | Residues 55- | KVSNRFS |
| | CDR-L2 | 61 of SEQ ID | (SEQ ID NO.:20) |
| | | NO.:4 | |
| | VL 7C6hum7 | Residues 94- | FQGSHVPYT |
| | CDR-L3 | 102 of SEQ ID | (SEQ ID NO.:21) |
| | | NO.:4 | |

*CDRs are underlined in humanized light and heavy chains.

Known human Ig sequences are disclosed, e.g., www.ncbi.nlm.nih.gov/entrez-/query.fcgi; www.atcc.org/phage/hdb.html; www.sciquest.com/; www.abcam.com/; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kuby05.htm; www.library.thinkquest.org/12429/Immune/Antibody.html; www.hhmi.org/grants/lectures/1996/vlab/; www.path.cam.ac.uk/.about.mrc7/m-ikeimages.html; www.antibodyresource.com/; mcb.harvard.edu/BioLinks/Immuno-logy.html.www.immunologylink.com/; pathbox.wustl.edu/.about.hcenter/index.-html; www.biotech.ufl.edu/.about.hcl/; www.pebio.com/pa/340913/340913.html-; www.nal.usda.gov/awic/pubs/antibody/; www.m.ehime-u.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.uni-marburg.de/.about.rek/AEP-Start.html; baserv.uci.kun.nl/.about.jraats/linksl.html; www.recab.uni-hd.de/immuno.bme.nwu.edu/; www.mrc-cpe.cam.ac.uk/imt-doc/pu-blic/INTRO.html; www.ibt.unam.mx/vir/V mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOsem-inar/SlideOl.html; www.cryst.bbk.ac.uk/.about.ubcgO7s/; www.nimr.mrc.ac.uk/CC/ccaewg/ccaewg.htm; www.path.cam.ac.uk/.about.mrc7/h-umanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Web-pages/Pept/spottech.html; www.jerini.de/fr roducts.htm; www.patents.ibm.com/ibm.html.Kabat et al., Sequences of Proteins of Immunological Interest, U.S. Dept. Health (1983), each entirely incorporated herein by reference. Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al., Nature 321:522 (1986); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska. et al., PNAS 91:969-973 (1994); International Appln. Publication No. WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

### C. Production of Antibodies and Antibody-Producing Cell Lines

As noted above, preferably, anti-Aβ(20-42) globulomer antibodies of the present invention or antibodies against any Aβ form that comprises the globulomer epitope with which the antibodies of the present invention are reactive exhibit a high capacity to reduce or to neutralize Aβ(20-42) globulomer (and/or any other Aβ form that comprises the globulomer epitope with which the antibodies of the present invention are reactive) activity, e.g., as assessed by any one of several in vitro and in vivo assays known in the art (e.g., see examples below).

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

Replacements of amino acid residues in the Fc portion to alter antibody effector function are known in the art (Winter, et al. U.S. Pat. Nos. 5,648,260 and 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment, at least one amino acid residue is replaced in the constant region of the antibody, for example the Fc region of the antibody, such that effector functions of the antibody are altered.

One embodiment provides a labeled binding protein wherein an antibody or antibody portion of the invention is derivatized or linked to another functional molecule (e.g., another peptide or protein). For example, a labeled binding protein of the invention can be derived by functionally linking an antibody or antibody portion of the invention (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

Another embodiment of the invention provides a crystallized binding protein. Preferably, the invention relates to crystals of whole anti-Aβ(20-42) globulomer antibodies and fragments thereof as disclosed herein, and formulations and compositions comprising such crystals. In one embodiment the crystallized binding protein has a greater half-life in vivo than the soluble counterpart of the binding protein. In another embodiment, the binding protein retains biological activity after crystallization.

Crystallized binding protein of the invention may be produced according methods known in the art and as disclosed in International Appln. Publication No. WO 02/072636, incorporated herein by reference.

Another embodiment of the invention provides a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick, S. C., et al., Exp. Med. (1988) 168:1099-1109; Wright, A., et al., EMBO J. (1991) 10:2717 2723).

One aspect of the present invention is directed to generating glycosylation site mutants in which the O- or N-linked glycosylation site of the binding protein has been mutated. One skilled in the art can generate such mutants using standard well-known technologies. The creation of glycosylation site mutants that retain the biological activity but have increased or decreased binding activity are another object of the present invention.

In still another embodiment, the glycosylation of the antibody or antigen-binding portion of the invention is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in International Appln. Publication No. WO 03/016466A2, and U.S. Pat. Nos. 5,714,350 and 6,350,861, each of which is incorporated herein by reference in its entirety.

Additionally or alternatively, a modified antibody of the invention can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent NO.: EP 1,176,195; International Appln. Publication Nos. WO 03/035835 and WO 99/54342 80, each of which is incorporated herein by reference in its entirety.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. Preferably the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S Patent Application Publication Nos. 20040018590 and 20020137134 and International Appln. Publication No. WO 05/100584 A2).

The term “multivalent binding protein” is used in this specification to denote a binding protein comprising two or more antigen binding sites. The multivalent binding protein is preferably engineered to have the three or more antigen binding sites, and is generally not a naturally occurring antibody. The term “multispecific binding protein” refers to a binding protein capable of binding two or more related or unrelated targets. Dual variable domain (DVD) binding proteins as used herein, are binding proteins that comprise two or more antigen binding sites and are tetravalent or multivalent binding proteins. Such DVDs may be monospecific, i.e capable of binding one antigen or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. DVD binding proteins and methods of making DVD binding proteins are disclosed in U.S. patent application Ser. No. 11/507,050 and incorporated herein by reference.

One aspect of the invention pertains to a DVD binding protein comprising binding proteins capable of binding to Aβ(20-42) globulomer. Preferably, the DVD binding protein is capable of binding Aβ(20-42) globulomer and/or any other Aβ form that comprises the globulomer epitope with which the antibodies of the present invention are reactive and a second target.

In addition to the binding proteins, the present invention is also directed to an anti-idiotypic (anti-Id) antibody specific for such binding proteins of the invention. An anti-Id antibody is an antibody, which recognizes unique determinants generally associated with the antigen-binding region of another antibody. The anti-Id can be prepared by immunizing an animal with the binding protein or a CDR containing region thereof. The immunized animal will recognize, and respond to the idiotypic determinants of the immunizing antibody and produce an anti-Id antibody. The anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. Preferably, the protein having a particularly selected novel glycosylation pattern exhibits improved or altered biological properties.

### D. Uses of Anti-Aβ(20-42) Antibodies

Given their ability to bind to Aβ(20-42) globulomer, the anti-Aβ(20-42) globulomer antibodies or antibodies against any Aβ form that comprises the globulomer epitope with which the antibodies of the present invention are reactive, or portions thereof, of the invention can be used to detect Aβ(20-42) globulomer and/or any other Aβ form that comprises the globulomer epitope with which the antibodies of the present invention are reactive (e.g., in a biological sample such as serum, CSF, brain tissue or plasma), using a conventional immunoassay, such as an enzyme linked immunosorbent assays (ELISA), an radioimmunoassay (RIA) or tissue immunohistochemistry. The invention therefore provides a method for detecting Aβ(20-42) globulomer and/or any other Aβ form that comprises the globulomer epitope with which the antibodies of the present invention are reactive in a biological sample comprising contacting a biological sample with an antibody, or antibody portion, of the invention and detecting either the antibody (or antibody portion) bound to Aβ(20-42) globulomer (and/or any other Aβ form that comprises the globulomer epitope with which the antibodies of the present invention are reactive) or unbound antibody (or antibody portion), to thereby detect Aβ(20-42) globulomer and/or any other Aβ form that comprises the globulomer epitope with which the antibodies of the present invention are reactive, in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Alternative to labeling the antibody, Aβ(20-42) globulomer and/or any other Aβ form that comprises the globulomer epitope with which the antibodies of the present invention are reactive can be assayed in biological fluids by a competition immunoassay utilizing recombinant Aβ(20-42) globulomer standards labeled with a detectable substance and an unlabeled anti-Aβ(20-42) globulomer antibody. In this assay, the biological sample, the labeled recombinant Aβ(20-42) globulomer standards and the anti-Aβ(20-42) globulomer antibody are combined, and the amount of labeled recombinant Aβ(20-42) globulomer standard bound to the unlabeled antibody is determined. The amount of Aβ(20-42) globulomer and/or any other Aβ form that comprises the globulomer epitope with which the antibodies of the present invention are reactive, in the biological sample, is inversely proportional to the amount of labeled rAβ(20-42) globulomer standard bound to the anti-Aβ(20-42) globulomer antibody.

The antibodies and antibody portions of the invention preferably are capable of neutralizing Aβ(20-42) globulomer activity and/or activity of any other Aβ form that comprises the globulomer epitope with which the antibodies of the present invention are reactive, both in vitro and in vivo. Accordingly, such antibodies and antibody portions of the invention can be used to inhibit Aβ(20-42) globulomer activity and/or activity of any other Aβ form that comprises the globulomer epitope with which the antibodies of the present invention are reactive, e.g., in a cell culture containing Aβ(20-42) globulomer and/or any other Aβ form that comprises the globulomer epitope with which the antibodies of the present invention are reactive, in human subjects, or in other mammalian subjects having Aβ(20-42) globulomer and/or any other Aβ form that comprises the globulomer epitope with which the antibodies of the present invention are reactive, with which an antibody of the invention cross-reacts. In one embodiment, the invention provides a method for inhibiting Aβ(20-42) globulomer activity and/or activity of any other Aβ form that comprises the globulomer epitope with which the antibodies of the present invention are reactive, comprising contacting Aβ(20-42) globulomer and/or and/or any other Aβ form that comprises the globulomer epitope with which the antibodies of the present invention are reactive, with an antibody or antibody portion of the invention such that Aβ(20-42) globulomer activity and/or activity of any other Aβ form that comprises the globulomer epitope with which the antibodies of the present invention are reactive is inhibited. For example, in a cell culture containing or suspected of containing Aβ(20-42) globulomer and/or any other Aβ form that comprises the globulomer epitope with which the antibodies of the present invention are reactive, an antibody or antibody portion of the invention can be added to the culture medium to inhibit Aβ(20-42) globulomer activity and/or activity of any other Aβ form that comprises the globulomer epitope with which the antibodies of the present invention are reactive in the culture.

In another embodiment, the invention provides a method for reducing Aβ(20-42) globulomer activity and/or reducing activity of any other Aβ form that comprises the globulomer epitope with which the antibodies of the present invention are reactive in a subject, advantageously from a subject suffering from a disease or disorder in which Aβ(20-42) globulomer activity is detrimental and/or activity of any other Aβ form that comprises the globulomer epitope with which the antibodies of the present invention are reactive is detrimental (e.g., an amyloidosis such as Alzheimer's Disease). The invention therefore provides methods for reducing Aβ(20-42) globulomer activity and/or activity of any other Aβ form that comprises the globulomer epitope with which the antibodies of the present invention are reactive, in a subject suffering from such a disease or disorder, which method comprises administering to the subject an antibody or antibody portion of the invention such that Aβ(20-42) globulomer activity and/or activity of any other Aβ form that comprises the globulomer epitope with which the antibodies of the present invention are reactive in the subject is reduced. Preferably, the Aβ(20-42) globulomer is human Aβ(20-42) globulomer and/or any other human Aβ form that comprises the globulomer epitope with which the antibodies of the present invention are reactive, and the subject is a human subject. Alternatively, the subject can be a mammal expressing APP or any Aβ-form resulting in the generation of Aβ(20-42) globulomer and/or any other Aβ form that comprises the globulomer epitope with which the antibodies of the present invention are reactive, to which an antibody of the invention is capable of binding. Still further, the subject can be a mammal into which Aβ(20-42) globulomer (and/or any other Aβ form that comprises the globulomer epitope with which the antibodies of the present invention are reactive) has been introduced (e.g., by administration of Aβ(20-42) globulomer or by expression of APP or any other Aβ-form resulting in the generation of Aβ(20-42) globulomer and/or any other Aβ form that comprises the globulomer epitope with which the antibodies of the present invention are reactive). An antibody of the invention can be administered to a human subject for therapeutic purposes. Moreover, an antibody of the invention can be administered to a non-human mammal wherein expression of APP or any Aβ-form resulting in the generation of Aβ(20-42) globulomer (and/or any other Aβ form that comprises the globulomer epitope with which the antibodies of the present invention are reactive) and/or with which the antibody is capable of binding for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which Aβ(20-42) globulomer activity and/or any other Aβ-form that comprises the globulomer epitope with which the antibodies of the present invention are reactive is detrimental” is intended to include diseases and other disorders in which the presence of Aβ(20-42) globulomer and/or any other Aβ form that comprises the globulomer epitope with which the antibodies of the present invention are reactive in a subject suffering from the disorder has been shown to be or is suspected of being either responsible for the pathophysiology of the disorder or a factor that contributes to a worsening of the disorder. Accordingly, a disorder in which Aβ(20-42) globulomer activity and/or activity of any Aβ form that comprises the globulomer epitope with which the antibodies of the present invention are reactive is detrimental is a disorder in which reduction of Aβ(20-42) globulomer activity and/or activity of any Aβ form that comprises the globulomer epitope with which the antibodies of the present invention are reactive is expected to alleviate some or all of the symptoms and/or progression of the disorder. Such disorders may be evidenced, for example, by an increase in the concentration of Aβ(20-42) globulomer and/or any Aβ form that comprises the globulomer epitope with which the antibodies of the present invention are reactive, in a biological fluid of a subject suffering from the disorder (e.g., an increase in the concentration of Aβ(20-42) globulomer and/or any Aβ form that comprises the globulomer epitope with which the antibodies of the present invention are reactive in serum, brain tissue, plasma, cerebrospinal fluid, etc. of the subject), which can be detected, for example, using an anti-Aβ(20-42) globulomer antibody and/or antibody against any other Aβ form that comprises the globulomer epitope with which the antibodies of the present invention are reactive, as described above or any antibody to any Aβ form that comprises the globulomer epitope with which the antibodies of the present invention are reactive. Non-limiting examples of disorders that can be treated with the antibodies of the invention include those disorders discussed in the section below pertaining to pharmaceutical compositions of the antibodies of the invention.

### D. Pharmaceutical Composition

The invention also provides pharmaceutical compositions comprising an antibody, or antigen-binding portion thereof, of the invention and a pharmaceutically acceptable carrier. The pharmaceutical compositions comprising antibodies of the invention are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies of the invention. In another embodiment, the pharmaceutical composition comprises one or more antibodies of the invention and one or more prophylactic or therapeutic agents other than antibodies of the invention for treating a disorder in which Aβ(20-42) globulomer activity is detrimental or activity of any Aβ form that comprises the globulomer epitope with which the antibodies of the present invention are reactive is detrimental. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and International Appln. Publication Nos. WO 92/19244, WO 97/32572, WO 97/44013, WO 98/31346, and WO 99/66903, each of which is incorporated herein by reference their entireties. In one embodiment, an antibody of the invention, combination therapy, or a composition of the invention is administered using Alkermes AIR® pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Appln. Publication No. WO 99/15154; and International Appln. Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, International Appln. Publication No. WO 91/05548, International Appln. Publication No. WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol, the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and International Appln. Publication Nos. WO 92/19244, WO 97/32572, WO 97/44013, WO 98/31346, and WO 99/66903, each of which is incorporated herein by reference their entireties. In a specific embodiment, an antibody of the invention, combination therapy, and/or composition of the invention is administered using Alkermes AIR® pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The antibodies and antibody portions of the invention can be incorporated into a pharmaceutical composition suitable for parenteral administration. Preferably, the antibody or antibody portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the antibodies and antibody-portions of the invention prepared as an injectable solution for parenteral administration, can further comprise an agent useful as an adjuvant, such as those used to increase the absorption, or dispersion of a therapeutic protein (e.g., antibody). A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions. (See International Appln. Publication No. WO 04/078140 and U.S. Patent Appln. Publication No. US2006104968, incorporated herein by reference.)

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies and antibody portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is coformulated with and/or coadministered with one or more additional therapeutic agents that are useful for treating disorders in which Aβ(20-42) activity is detrimental. For example, an anti-Aβ(20-42) antibody or antibody portion of the invention may be coformulated and/or coadministered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an antibody to Aβ(20-42) or fragment thereof (or an antibody to any other Aβ form that comprises the globulomer epitope with which the antibodies of the present invention are reactive) is linked to a half-life extending vehicle known in the art. Such vehicles include, but are not limited to, the Fc domain, polyethylene glycol, and dextran. Such vehicles are described, e.g., in U.S. patent application Ser. No. 09/428,082 and published International Patent Application No. WO 99/25044, which are hereby incorporated by reference for any purpose.

In a specific embodiment, nucleic acid sequences comprising nucleotide sequences encoding an antibody of the invention or another prophylactic or therapeutic agent of the invention are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the invention, the nucleic acids produce their encoded antibody or prophylactic or therapeutic agent of the invention that mediates a prophylactic or therapeutic effect.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990). Detailed description of various methods of gene therapy are disclosed in U.S. Patent Application Publication No. US20050042664 A1 which is incorporated herein by reference.

Antibodies of the invention or antigen binding portions thereof can be used alone or in combination to treat diseases such as Alzheimer's Disease, Down's Syndrome, dementia, Parkinson's Disease, or any other disease or condition associated with a build up of amyloid beta protein within the brain. The antibodies of the present invention may be used to treat “conformational diseases”. Such diseases arise from secondary to tertiary structural changes within constituent proteins with subsequent aggregation of the altered proteins (Hayden et al., JOP. J Pancreas 2005; 6(4):287-302). In particular, the antibodies or binding proteins of the present invention may be used to treat one or more of the following conformational diseases: Alpha1-antitrypsin-deficiency, C1-inhibitor deficiency angioedema, Antithrombin deficiency thromboembolic disease, Kuru, Creutzfeld-Jacob disease/scrapie, Bovine spongiform encephalopathy, Gerstmann-Straussler-Scheinker disease, Fatal familial insomnia, Huntington's disease, Spinocerebellar ataxia, Machado-Joseph atrophy, Dentato-rubro-pallidoluysian atrophy, Frontotemporal dementia, Sickle cell anemia, Unstable hemoglobin inclusion-body hemolysis, Drug-induced inclusion body hemolysis, Parkinson's disease, Systemic AL amyloidosis, Nodular AL amyloidosis, Systemic AA amyloidosis, Prostatic amyloid, Hemodialysis amyloidosis, Hereditary (Icelandic) cerebral angiopathy, Huntington's disease, Familial visceral amyloid, Familial visceral polyneuropathy, Familial visceral amyloidosis, Senile systemic amyloidosis, Familial amyloid neuropathy, Familial cardiac amyloid, Alzheimer's disease, Down's syndrome, Medullary carcinoma thyroid and Type 2 diabetes mellitus (T2DM). Preferably, the antibodies of the present invention may be utilized to treat an amyloidosis, for example, Alzheimer's disease and Down's syndrome.

It should be understood that the antibodies of the invention or antigen binding portion thereof can be used alone or in combination with one or more additional agents, e.g., a therapeutic agent (for example, a small molecule or biologic), said additional agent being selected by the skilled artisan for its intended purpose. For example, the additional agent can be a therapeutic agent such as a cholesterinase inhibitor (e.g., tactrine, donepezil, rivastigmine or galantamine), a partial NMDA receptor blocker (e.g., memantine), a glycosaminoglycan mimetic (e.g., Alzhemed), an inhibitor or allosteric modulator of gamma secretase (e.g., R-flurbiprofen), a luteinizing hormone blockade gonadotropin releasing hormone agonist (e.g., leuprorelin), a serotinin 5-HT1A receptor antagonist, a chelatin agent, a neuronal selective L-type calcium channel blocker, an immunomodulator, an amyloid fibrillogenesis inhibitor or amyloid protein deposition inhibitor (e.g., M266), another antibody (e.g., bapineuzumab), a 5-HT1a receptor antagonist, a PDE4 inhibitor, a histamine agonist, a receptor protein for advanced glycation end products, a PARP stimulator, a serotonin 6 receptor antagonist, a 5-HT4 receptor agonist, a human steroid, a glucose uptake stimulant which enhances neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator (i.e., those agents currently recognized, or in the future being recognized, as useful to treat the disease or condition being treated by the antibody of the present invention). The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### Example I

### Preparation of Globulomers

### a) Aβ(1-42) Globulomer:

The Aβ(1-42) synthetic peptide (H-1368, Bachem, Bubendorf, Switzerland) was suspended in 100% 1,1,1,3,3,3-hexafluoro-2-propanol (HFIP) at 6 mg/mL and incubated for complete solubilization under shaking at 37° C. for 1.5 h. The HFIP acts as a hydrogen-bond breaker and is used to eliminate pre-existing structural inhomogeneities in the Aβ peptide. HFIP was removed by evaporation in a SpeedVac and Aβ(1-42) resuspended at a concentration of 5 mM in dimethylsulfoxide and sonicated for 20 s. The HFIP-pre-treated Aβ(1-42) was diluted in phosphate-buffered saline (PBS) (20 mM NaH₂PO₄, 140 mM NaCl, pH 7.4) to 400 μM and 1/10 volume 2% sodium dodecyl sulfate (SDS) (in H₂O) added (final concentration of 0.2% SDS). An incubation for 6 h at 37° C. resulted in the 16/20-kDa Aβ(1-42) globulomer (short form for globular oligomer) intermediate. The 38/48-kDa Aβ(1-42) globulomer was generated by a further dilution with three volumes of H₂O and incubation for 18 h at 37° C. After centrifugation at 3000 g for 20 min the sample was concentrated by ultrafiltration (30-kDa cut-off), dialysed against 5 mM NaH₂PO₄, 35 mM NaCl, pH 7.4, centrifuged at 10000 g for 10 min and the supernatant comprising the 38/48-kDa Aβ(1-42) globulomer withdrawn. As an alternative to dialysis the 38/48-kDa Aβ(1-42) globulomer could also be precipitated by a ninefold excess (v/v) of ice-cold methanol/acetic acid solution (33% methanol, 4% acetic acid) for 1 h at 4° C. The 38/48-kDa Aβ(1-42) globulomer is then pelleted (10 min at 16200 g), resuspended in 5 mM NaH₂PO₄, 35 mM NaCl, pH 7.4, and the pH adjusted to 7.4.

### b) Aβ(20-42) Globulomer:

1.59 ml of Aβ(1-42) globulomer preparation prepared according to Example Ia were admixed with 38 ml of buffer (50 mM MES/NaOH, pH 7.4) and 200 μl of a 1 mg/ml thermolysin solution (Roche) in water. The reaction mixture was stirred at RT for 20 h. Then, 80 μl of a 100 mM EDTA solution, pH 7.4, in water were added and the mixture was furthermore adjusted to an SDS content of 0.01% with 400 μl of a 1% strength SDS solution. The reaction mixture was concentrated to approx. 1 ml via a 15 ml 30 kDa Centriprep tube. The concentrate was admixed with 9 ml of buffer (50 mM MES/NaOH, 0.02% SDS, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 2% strength SDS solution in water. The sample was centrifuged at 10000 g for 10 min and the Aβ(20-42) globulomer supernatant was withdrawn.

### c) Aβ(12-42) Globulomer:

2 ml of an Aβ(1-42) globulomer preparation prepared according to Example 1a were admixed with 38 ml buffer (5 mM sodium phosphate, 35 mM sodium chloride, pH 7.4) and 150 μl of a 1 mg/ml GluC endoproteinase (Roche) in water. The reaction mixture was stirred for 6 h at RT, and a further 150 μl of a 1 mg/ml GluC endoproteinase (Roche) in water were subsequently added. The reaction mixture was stirred at RT for another 16 h, followed by addition of 8 μl of a 5 M DIFP solution. The reaction mixture was concentrated to approx. 1 ml via a 15 ml 30 kDa Centriprep tube. The concentrate was admixed with 9 ml of buffer (5 mM sodium phosphate, 35 mM sodium chloride, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 1% strength SDS solution in water. The sample was centrifuged at 10000 g for 10 min and the Aβ(12-42) globulomer supernatant was withdrawn.

### d) Cross-Linked Aβ(1-42) Globulomer:

The Aβ(1-42) synthetic peptide (H-1368, Bachem, Bubendorf, Switzerland) was suspended in 100% 1,1,1,3,3,3-hexafluoro-2-propanol (HFIP) at 6 mg/ml and incubated for complete solubilization under shaking at 37 degrees Celsius for 1.5 h. The HFIP acts as a hydrogen-bond breaker and was used to eliminate pre-existing structural inhomogeneities in the Aβ peptide. HFIP was removed by evaporation by a SpeedVac and Aβ(12-42) globulomer Aβ(1-42) resuspended at a concentration of 5 mM in dimethylsulfoxide and sonicated for 20 s. The HFIP-pre-treated Aβ(1-42) was diluted in PBS (20 mM NaH2PO4, 140 mM NaCl, pH 7.4) to 400 uM and 1/10 vol. 2% SDS (in water) added (final conc. Of 0.2% SDS). An incubation for 6 h at 37 degrees Celsius resulted in the 16/20-kDa Aβ(1-42) globulomer (short form for globulomer oligomer) intermediate. The 38/48-kDa Aβ(1-42) globulomer was generated by a further dilution with 3 volumes of water and incubation for 18 h at 37 degrees Celsius. Cross-linking of the 38/48-kDa Aβ(1-42) globulomer was now performed by incubation with 1 mM glutaraldehyde for 2 h at 21 degrees Celsius room temperature followed by ethanolamine (5 mM) treatment for 30 minutes at room temperature.

### Example II

### Generation and Isolation of Humanized Anti-Aβ(20-42) Globulomer Monoclonal Antibodies

### Preparation of Humanized Antibodies:

For humanization of the 5F7 variable regions, the general approach provided in the present invention was followed. First, a molecular model of the 5F7 variable regions was constructed with the aid of the computer programs ABMOD and ENCAD (Levitt, M., J. Mol. Biol. 168: 595-620 (1983)). Next, based on a homology search against human V and J segment sequences, the VH segment MUC1-1′ CL (Griffiths, A. D., et al., EMBO J. 12: 725-734 (1993)) and the J segment JH4 (Ravetch, J. V., et al., Cell 27: 583-591 (1981)) were selected to provide the frameworks for the Hu5F7 heavy chain variable region. For the Hu5F7 light chain variable region, the VL segment TR1.37′ CL (Portolano, S., et al., J. Immunol. 151: 2839-2851 (1993)) and the J segment JK4 (Hieter, P. A., et al., J. Biol. Chem. 257: 1516-1522 (1982)) were used. The identity of the framework amino acids between 5F7 VH and the acceptor human MUC1-1′ CL and JH4 segments was 78%, while the identity between 5F7 VL and the acceptor human TR1.37′ CL and JK4 segments was 86%.

At framework positions in which the computer model suggested significant contact with the CDRs, the amino acids from the mouse V regions were substituted for the original human framework amino acids. This was done at residues 48, 67, 68, 70 and 72 for the heavy chain (FIG. 7), and at position 7 for the light chain (FIG. 8). Framework residues that occurred only rarely at their respective positions in the corresponding human V region subgroups were replaced with human consensus amino acids at those positions. This was done at residue 76 of the heavy chain (FIG. 7), and at residues 1 and 2 of the light chain (FIG. 8).

The humanization design strategy for 7C6 followed a similar approach resulting in the sequences SEQ ID NO.:3 for the heavy chain and SEQ ID NO.:4 for the light chain.

### Assembly of Humanized Antibody VH and VL Fragments.

VH and VL gene fragments for the 5F7 and 7C6 humanization designs (SEQ ID NO.:1 and 2 for 5F7hum8 and SEQ ID NO.:3 and 4 for 7C6hum7) were assembled by annealing overlapping oligonucleotides covering the entire sequence. Briefly, the entire coding strand of the VH or VL fragment was divided into a series of sixty-nucleotide oligos, each designed to have a thirty nucleotide overlap with two corresponding bottom strand oligos. The sum of the bottom strand oligos also covered the entire sequence. Taken together, the oligonucleotides filled the complete double-stranded DNA segment.

In the first step of the procedure, the oligonucleotides were kinased (New England Biolabs cat #201S) by combining seven top strand and seven bottom strand oligos together at a concentration of 3 nM each in a 100 microliter reaction for 30 minutes at 37° C. The kinased oligos were then phenol/chloroform extracted, precipitated, and resuspended in 100 microliters of NEB Ligase Buffer.

In the second step of the procedure, the oligonucleotides were annealed by heating to 95° C., then slowly cooled to 20° C. over a period of 90 minutes by a controlled cooling ramp in a PCR machine.

In the third step of the procedure, 1 microliter of Ligase (NEB cat#202S) was added to the annealed oligos in order to ligate them together to form the strands of the VH and VL segments. Ligase was inactivated by heating to 65° C. for 10 minutes.

In the fourth step, the ends of the assembled fragments were filled in with Klenow enzyme (NEB cat#212S), and the DNA was gel purified before cloning into the human heavy and light chain cassette vectors already containing heavy chain constant region sequences encoding a peptide sequence according to SEQ ID NO.:38 (for “wt” constructs) or SEQ ID NO.:39 (for “mut” constructs) or light chain constant region sequences encoding a peptide sequence according to SEQ ID NO.:40 or SEQ ID NO.:41, respectively.

### Example III

### Characterization of Generated Antibodies

### Competition ELISA

The following protocol was utilized to carry out the Competition ELISA assay:

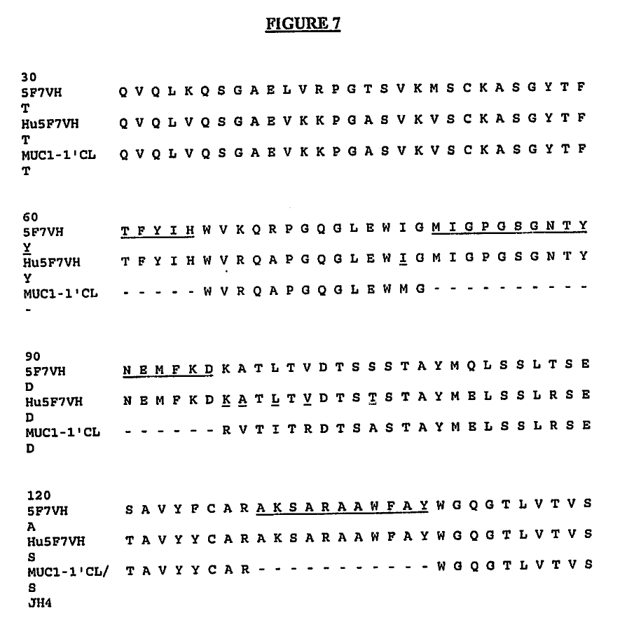

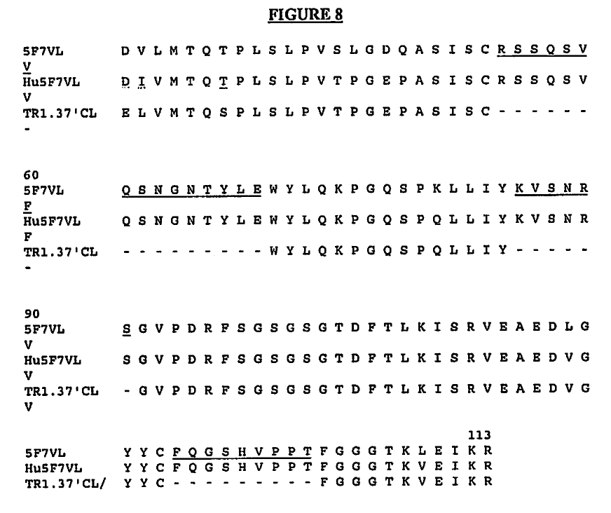

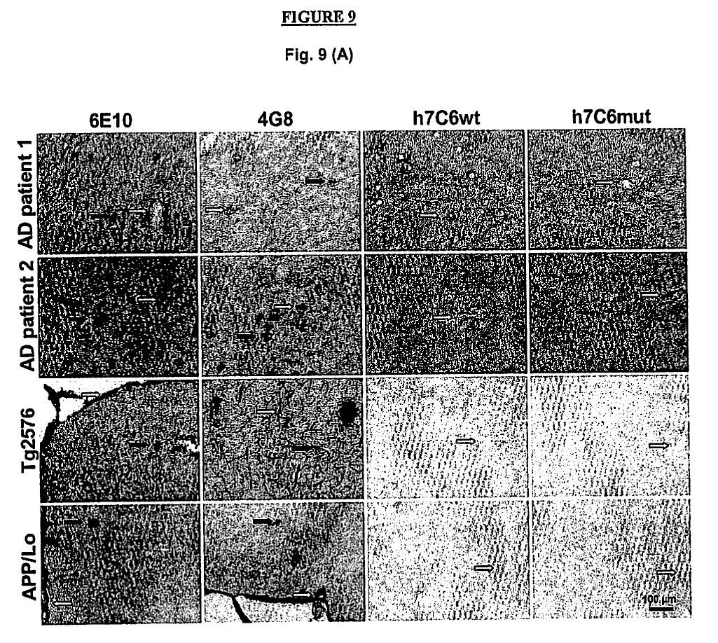

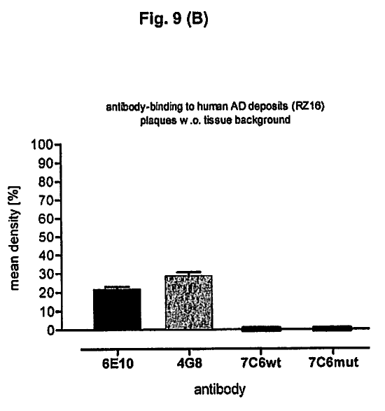

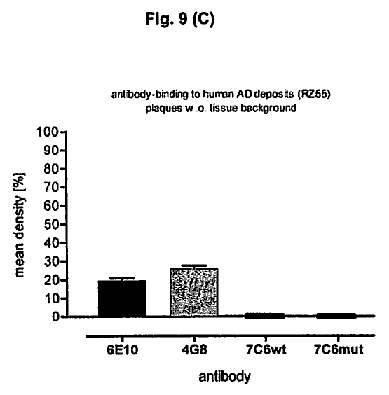

Initially, the plates (1 plate/experiment) were coated overnight with A-Beta antigen (1-42) at a concentration of 5 μg/mL in phosphate buffered saline (PBS). The following day, the supernatant was discarded, and the plates were blocked with 340 mL of Super Block buffer (Pierce, Rockford, Ill.) for 45 min. The plates were then emptied, and the biotinylated 7C6 or 5F7 mouse antibody was added at a concentration of 1 μg/mL. (Volume=100 μL) Other antibodies (mouse or humanized 5F7; or mouse or humanized 7C6) were added at concentrations ranging from 27 μg/mL to 0.11 μg/mL. (Volume=50 μL) The plates were then incubated for two hours and washed 5× times with Phosphate Buffered Saline (PBS). Neutra Avidin HRP was added as a secondary reagent (dilution 1:20,000; volume=100 μL). The plates were then incubated for 30 min. and washed 5× times. TMB (Invitrogen, Carlsbad, Calif.) substrate was then added (volume=100 μL). Subsequently, the plates were incubated for 4 min. The reaction was then stopped with 2N sulfuric acid (volume=100 μL). Plates were read spectrophotometrically at a wavelength of 450 nm. The results are shown in FIGS. 3 and 4.

In particular, FIG. 3 shows the equivalence of humanized antibody 5F7 to the mouse parent antibody in connection with its ability to compete with (and inhibit the binding signal of) the biotinylated mouse antibody. Thus, the humanized antibody retained its binding potency.

FIG. 4 shows the equivalence of humanized antibody 7C6 to the mouse parent antibody with respect to its ability to compete with (and inhibit the binding signal of) the biotinylated mouse antibody. Again, the humanized antibody retained its binding potency.

### Example IV

### Aβ(20-42) Globulomer Selectivity of the Antibodies

### Example IV.1

### Semi-Quantitative Analysis Visualized by SDS-PAGE of the Discrimination of Aβ(20-42) Globulomer Selective Antibodies for Aβ(1-42) Fibrils

### A) Aβ(1-42) Fibril Preparation:

1 mg of Aβ(1-42) (Bachem, Cat. no.: H-1368) were dissolved in 500 μl 0.1% NH₄OH in H₂O and agitated for 1 min at ambient temperature. The sample was centrifuged for 5 min at 10′000 g. The supernatant was collected. Aβ(1-42) concentration in the supernatant was determined according to Bradford's method (BIO-RAD).

100 μl of Aβ(1-42) in 0.1% NH₄OH were mixed with 300 μl of 20 mM NaH₂PO₄, 140 mM NaCl, pH 7.4 and adjusted to pH 7.4 with 2% HCl. The sample was then incubated at 37° C. for 20 hours. Then, the sample was centrifuged for 10 min at 10′000 g. The supernatant was discarded, and the residue was mixed with 400 μl of 20 mM NaH₂PO₄, 140 mM NaCl, pH 7.4, resuspended by vigorous agitation (“vortexing”) for 1 min and centrifuged for 10 min at 10′000 g. The supernatant was discarded and the residue was mixed with 400 μl of 20 mM NaH₂PO₄, 140 mM NaCl, pH 7.4, resuspended by vigorous agitation (“vortexing”) for 1 min and centrifuged for 10 min at 10′000 g once more. The supernatant was discarded. The residue was resuspended in 380 μl of 20 mM NaH₂PO₄, 140 mM NaCl, pH 7.4 and prompted by vigorous agitation (“vortexing”).

### B) Binding of Anti-Aβ Antibodies to Aβ(1-42) Fibrils:

80 μl of Aβ(1-42) fibril preparation were diluted with 320 μl of 20 mM NaH₂PO₄, 140 mM NaCl, 0.05% Tween 20, pH 7.4, agitated 5 min at ambient temperature, followed by sonification (20 sec), then the sample was centrifuged for 10 min at 10′000 g. The supernatant was discarded, and the residue was resuspended in 190 μl of 20 mM NaH₂PO₄, 140 mM NaCl, 0.05% Tween 20, pH 7.4. Resuspension was prompted by vigorous agitation (“vortexing”). Aliquots of 10 μl of the fibril preparation were each mixed with:
a) 10 μl 20 mM NaH₂PO₄, 140 mM NaCl, pH 7.4
b) 10 μl 0.5 μg/μl of 5F7hum8 in 20 mM NaH₂PO₄, 140 mM NaCl, pH 7.4
c) 10 μl 0.5 μg/μl of 7C6hum7mut in 20 mM NaH₂PO₄, 140 mM NaCl, pH 7.4
d) 10 μl 0.5 μg/μl of 7C6hum7 wt in 20 mM NaH₂PO₄, 140 mM NaCl, pH 7.4
e) 10 μl 0.5 μg/μl of 6E10 (Signet Nr.: 9320) in 20 mM NaH₂PO₄, 140 mM NaCl, pH 7.4
f) 10 μl 0.5 μg/μl of IgG2a (i.e., antibody isotype control made against KLH (Keyhole Limpet Hemocyanin) as antigen) in 20 mM NaH₂PO₄, 140 mM NaCl, pH 7.4

The samples were incubated at 37° C. for 20 hours, then centrifuged for 10 min at 10′000 g. The supernatants were collected and mixed with 20 μl of SDS-PAGE sample buffer. The residues were mixed with 50 μl of 20 mM NaH₂PO₄, 140 mM NaCl, 0.025% Tween 20, pH 7.4 and resuspended by “vortexing”, and then the samples were centrifuged for 10 min at 10′000 g. The supernatants were discarded, and the residues were mixed with 20 μl 20 mM NaH₂PO₄, 140 mM NaCl, 0.025% Tween 20, pH 7.4, then with 20 μl of SDS-PAGE sample buffer. The samples were heated 5 min at 98° C. and applied to an 18% Tris/glycine gel for electrophoresis.

### Parameters for SDS-PAGE:

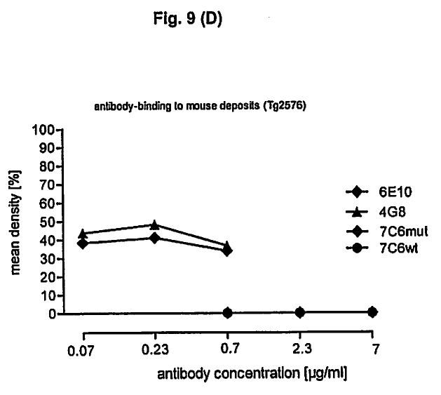

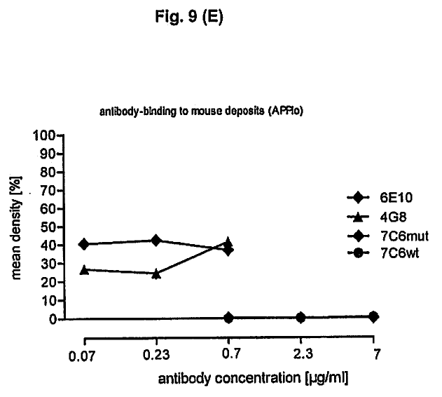

SDS sample buffer 0.3 g SDS
   0.77 g DTT
   4 ml 1 M Tris/HCl pH 6.8
   8 ml glycerine
   1 ml 1% bromphenol blue in ethanol
      Fill with H₂O and 50 ml 18% Tris/Glycine Gel: (Invitrogen, Cat. no.: EC6505BOX)
Electrophoresis buffer 7.5 g Tris
   36 g Glycine
   2.5 g SDS
      Fill with H₂O ad 2.5 l.
      The gel is run at a constant current of 20 mA.
Staining of the gels Coomassie Blue R250
   Results are shown in FIG. 5(A).
   C) Semiquantitative Analysis of Different Anti-Aβ Antibodies and their Discrimination of Aβ(1-42) Fibrils.

Positions of antibodies, Aβ(1-42) fibrils and Aβ(1-42) monomers are marked at the edge of the gel. Due to their size, Aβ(1-42) fibrils cannot enter the SDS-PAGE gel and can be seen in the gel slot.
1. Marker
2. Aβ(1-42) fibril preparation; control
3. Aβ(1-42) fibril preparation; +mAb 5F7hum8; 20 h 37° C.; supernatant
4. Aβ(1-42) fibril preparation; +mAb 5F7hum8; 20 h 37° C.; pellet
5. Aβ(1-42) fibril preparation; +mAb 7C6hum7mut; 20 h 37° C.; supernatant
6. Aβ(1-42) fibril preparation; +mAb 7C6hum7mut; 20 h 37° C.; pellet
7. Aβ(1-42) fibril preparation; +mAb 7C6hum7 wt; 20 h 37° C.; supernatant
8. Aβ(1-42) fibril preparation; +mAb 7C6hum7 wt; 20 h 37° C.; pellet
9. Aβ(1-42) fibril preparation; +mAb 6E10; 20 h 37° C.; supernatant
10. Aβ(1-42) fibril preparation; +mAb 6E10; 20 h 37° C.; pellet
11. Aβ(1-42) fibril preparation; +mAb IgG2a; 20 h 37° C.; supernatant
12. Aβ(1-42) fibril preparation; +mAb IgG2a; 20 h 37° C.; pellet

The relative binding to fibril type Aβ was evaluated from SDS-PAGE analysis by measuring the Optical Density (OD) values from the Heavy Chain of the antibodies in the fibril bound (pellet-fraction) and the supernatant fractions after centrifugation. Antibodies that have bound to the Aβ fibrils should be co-pelleted with the Aβ-fibrils and therefore are found in the pellet fraction whereas non-Aβ-fibril bound (free) antibodies are found in the supernatant. The percentage of antibody bound to Aβ-fibrils was calculated according to the following formula:

Percent antibody bound to Aβ-fibrils=OD_{fibril fraction}×100%/(OD_{fibril fraction}+OD_{supernatant fraction})

This procedure was performed for the mAbs 6E10 (Signet, Cat. no.: 9320), 5F7hum8, 7C6hum7mut and 7C6hum7 wt and IgG2a.

In the Alzheimer disease brain, the Aβ fibrils are a major component of the total Aβ peptide pool. By attacking these fibrils by anti Aβ-antibodies, the risk of negative side effects is elevated due to a liberation of high amounts of Aβ which subsequently may increase the risk of microhaemorrhages. An increased risk for microhaemorrhages was observed in an active immunization approach with fibrillar aggregates of the Aβ peptide (Bennett and Holtzman, 2005, Neurology, 64, 10-12; Orgogozo J, Neurology, 2003, 61, 46-54; Schenk et al., 2004, Curr Opin Immunol, 16, 599-606).

In contrast to the commercially available antibody 6E10 (Signet 9320) which recognizes a linear Aβ-epitope between AA1-17, the Aβ(20-42) globulomer selective antibody 5F7hum8 (which actually has the lowest selectivity for Aβ(20-42) globulomers over other Aβ-forms) does not bind to Aβ(1-42) fibrils in an co-pelleting experiment (see FIG. 5(b)). This is shown by the fact that the 5F7hum8 antibody after an incubation with Aβ(1-42) fibrils remains after a pelleting step in the supernatant and is not co-pelleted because of being bound to the Aβ(1-42) fibrils. The same result was found for the 7C6hum7 wt and 7C6hum7mut. As a reference for unspecific binding and the intrinsic background of this method the unspecific antibody IgG2a was used as in internal control. (IgG2a was made against KLH (Keyhole Limpet Hemocyanin) as antigen.) The IgG2a antibody which is not directed against the Aβ peptide in any form shows a certain unspecific binding to Aβ fibrils.

### Example IV.2

### Dot-Blot Profile of the Selectivity of the Anti-Aβ(20-42) Globulomer Humanized Antibodies

In order to characterize the selectivity of the humanized monoclonal anti Aβ(20-42) globulomer antibodies they were probed for recognition with different Aβ-forms. To this end, serial dilutions of the individual Aβ(1-42) forms ranging from 100 pmol/μl to 0.01 pmol/μl in PBS supplemented with 0.2 mg/ml BSA were made. 1 μl of each sample was blotted onto a nitrocellulose membrane. For detection, the corresponding antibody was used (0.2 μg/ml). Immunostaining was done using peroxidase conjugated anti-mouse-IgG or anti-human-IgG and the staining reagent BM Blue POD Substrate (Roche).

### Aβ-Standards for Dot-Blot:

1. Aβ(1-42) monomer, 0.1% NH₄OH

1 mg Aβ(1-42) (Bachem Inc., cat. no. H-1368) were dissolved in 0.5 ml 0.1% NH₄OH in H₂O (freshly prepared) (=2 mg/ml) and immediately shaken for 30 sec at room temperature to get a clear solution. The sample was stored at −20° C. for further use.

2. Aβ(1-40) monomer, 0.1% NH₄OH

1 mg Aβ(1-40) (Bachem Inc., cat. no. H-1368) were dissolved in 0.5 ml 0.1% NH₄OH in H₂O (freshly prepared) (=2 mg/ml) and immediately shaken for 30 sec. at room temperature to get a clear solution. The sample was stored at −20° C. for further use.

3. Aβ(1-42) monomer, 0.1% NaOH

2.5 mg Aβ(1-42) (Bachem Inc., cat. no. H-1368) were dissolved in 0.5 ml 0.1% NaOH in H₂O (freshly prepared) (=5 mg/ml) and immediately shaken for 30 sec. at room temperature to obtain a clear solution. The sample was stored at −20° C. for further use.

4. Aβ(1-40) monomer, 0.1% NaOH

2.5 mg Aβ(1-40) (Bachem Inc., cat. no. H-1368) were dissolved in 0.5 ml 0.1% NaOH in H₂O (freshly prepared) (=5 mg/ml) and immediately shaken for 30 sec. at room temperature to obtain a clear solution. The sample was stored at −20° C. for further use.

5. Aβ(1-42) globulomer

The preparation of the Aβ(1-42) globulomer is described in Example Ia.

6. Aβ(12-42) globulomer

The preparation of the Aβ(12-42) globulomer is described in Example Ic.

7. Aβ(20-42) globulomer

The preparation of the Aβ(20-42) globulomer is described in Example Ib.

8. Aβ(1-42) fibrils

1 mg Aβ(1-42) (Bachem Inc. cat. no.: H-1368) were solved in 500 μl aqueous 0.1% NH₄OH (Eppendorff tube) and the sample was stirred for 1 min at room temperature. 100 μl of this freshly prepared Aβ(1-42) solution were neutralized with 300 μl 20 mM NaH₂PO₄; 140 mM NaCl, pH 7.4. The pH was adjusted to pH 7.4 with 1% HCl. The sample was incubated for 24 h at 37° C. and centrifuged (10 min at 10000 g). The supernatant was discarded and the fibril pellet resuspended with 400 μl 20 mM NaH₂PO₄; 140 mM NaCl, pH 7.4 by vortexing for 1 min.

9. sAPPα

Supplied by Sigma (cat. no. S9564; 25 μg in 20 mM NaH₂PO₄; 140 mM NaCl; pH 7.4). The sAPPα was diluted to 0.1 mg/ml (=1 pmol/μl) with 20 mM NaH₂PO₄, 140 mM NaCl, pH 7.4, 0.2 mg/ml BSA.

### Materials for Dot Blot:

Aβ-standards:
Serial dilution of Aβ antigens in 20 mM NaH₂PO₄, 140 mM NaCl, pH 7.4+0.2 mg/ml BSA
   1) 100 pmol/μl
   2) 10 pmol/μl
   3) 1 pmol/μl
   4) 0.1 pmol/μl
   5) 0.01 pmol/μl
   6) 0.001 pmol/μl

### Nitrocellulose:

Trans-Blot Transfer medium, Pure Nitrocellulose Membrane (0.45 μm); BIO-RAD

### Anti-Mouse-POD:

Cat NO.: 715-035-150 (Jackson Immuno Research)

### Anti-human-POD:

Cat NO.: 109-035-003 (Jackson Immuno Research)
   Detection reagent:
BM Blue POD Substrate, precipitating (Roche)

### Bovine Serum Albumin, (BSA):

Cat NO.: A-7888 (SIGMA)
   Blocking reagent:
5% low fat milk in TBS
   Buffer solutions:
TBS
25 mM Tris/HCl buffer pH 7.5
+150 mM NaCl
TTBS
25 mM Tris/HCl-buffer pH 7.5
+150 mM NaCl
+0.05% Tween 20
PBS+0.2 mg/ml BSA
20 mM NaH₂PO₄ buffer pH 7.4
+140 mM NaCl
+0.2 mg/ml BSA

### Antibody Solution I:

0.2 μg/ml antibody diluted in 20 ml 1% low fat milk in TBS
   Antibody solution II:
1:5000 dilution
Anti-Mouse-POD in 1% low fat milk in TBS for mouse antibodies (i.e. 6E10) or anti-human-POD in 1% low fat milk in TBS for humanized anti Aβ(20-42) globulomer antibodies i.e. 5F7hum8, 7C6hum7 wt and 7C6hum7mut

### Dot Blot Procedure:

1) 1 μl each of the different Aβ-standards (in their 6 serial dilutions) were dotted onto the nitrocellulose membrane in a distance of approximately 1 cm from each other.
2) The Aβ-standards dots were allowed to dry on the nitrocellulose membrane on air for at least 10 min at room temperature (RT) (=dot blot)
3) Blocking:
   The dot blot was incubated with 30 ml 5% low fat milk in TBS for 16 h at RT.
4) Washing:
   The blocking solution was discarded and the dot blot was incubated under shaking with 20 ml TTBS for 10 min at RT.
5) Antibody solution I:
   The washing buffer was discarded and the dot blot was incubated with antibody solution I for 2 h at RT
6) Washing:
   The antibody solution I was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
7) Antibody solution II:
   The washing buffer was discarded and the dot blot was incubated with antibody solution II 1 h at RT
8) Washing:
   The antibody solution II was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
9) Development:
   The washing solution was discarded. The dot blot was developed with 5 ml BM Blue POD Substrate for 10 min. The development was stopped by intense washing of the dot blot with H₂O. Quantitative evaluation was done using a densitometric analysis (GS800 densitometer (BioRad) and software package Quantity one, Version 4.5.0 (BioRad)) of the dot-intensity. Only dots were evaluated that had a relative density of greater than 20% of the relative density of the last optically unambiguously identified dot of the Aβ(20-42) globulomer. This threshold value was determined for every dot-blot independently. The calculated value indicates the relation between recognition of Aβ(20-42) globulomer and the respective Aβ form for the antibody given.

Results are shown in FIG. 6(A).

Dot blot analysis of the specificity of different anti-Aβ antibodies (mouse monoclonal 6E10, 5F7hum8, 7C6hum7 wt, 7C6hum7mut) towards different forms of Aβ. The humanized monoclonal antibodies tested were obtained (except for the commercial mouse monoclonal antibody 6E10) by active immunization of mice with Aβ(20-42) globulomer, followed by selection of the fused hybridoma cells and subsequent humanization. The individual Aβ forms were applied in serial dilutions and incubated with the respective antibodies for immune reaction.
1. Aβ(1-42) monomer, 0.1% NH₄OH
2. Aβ(1-40) monomer, 0.1% NH₄OH
3. Aβ(1-42) monomer, 0.1% NaOH
4. Aβ(1-40) monomer, 0.1% NaOH
5. Aβ(1-42) globulomer
6. Aβ(12-42) globulomer
7. Aβ(20-42) globulomer
8. Aβ(1-42) fibril preparation
9. sAPPα (Sigma); (first dot: 1 pmol)

The anti-Aβ(20-42) globulomer selective antibodies can be divided in 3 classes with respect to the discrimination of Aβ(1-42) globulomer and Aβ(12-42) globulomer. The first class comprising antibodies and their humanized representative 5F7hum8 recognizes preferentially Aβ(20-42) globulomer and to some extent Aβ(1-42) globulomer (and also Aβ(12-42) globulomer). The second class (of which there is no humanized antibody but only mouse monoclonal antibodies available to this date) comprise antibodies that recognize preferentially Aβ(20-42) globulomer and also recognize Aβ(12-42) globulomer but to a lesser extent and do not significantly recognize Aβ(1-42) globulomer. The third class comprises antibodies and their humanized representatives 7C6hum7 wt and 7C6hum7mut recognizes A-(20-42) globulomer but shows no significant recognition of the others. All three classes do not significantly recognize monomeric Aβ(1-42), monomeric Aβ(1-40), Aβ(1-42) fibrils or sAPPα.

### Example V

### In Situ Analysis of the Specific Reaction of Antibodies H7C6WT and H7C6MUT to Fibrillary Aβ Peptide in the Form of Aβ Plaques in Old TG2576 Mice and Aβ Amyloid in Meningeal Vessels

For these experiments, brain material of 19 month old Tg2576 mice (Hsiao et al., 1996, Science; 274(5284), 99-102), of 17 month old APP/Lo mice (Moechars et al., 1999) or autopsy material of two Alzheimer's disease patients (RZ16 and RZ55; obtained from BrainNet, Munich) was used. The mice overexpress human APP with the so-called Swedish mutation (K670N/M671L) in the case of Tg2576 or human APP with the so-called London mutation (V717I) in the case of APP/Lo and formed β amyloid deposits in the brain parenchyma at about 11 months of age and β amyloid deposits in larger cerebral vessels at about 15-18 months of age. The animals were deeply anaesthetized and transcardially perfused with 0.1 M phosphate-buffered saline (PBS) to flush the blood. Then, the brain was removed from the cranium and divided longitudinally. One hemisphere of the brain was shock-frozen and the other fixated by immersion into 4% paraformaldehyde. The immersion-fixated hemisphere was cryoprotected by soaking in 30% sucrose in PBS and mounted on a freezing microtome. The entire forebrain was cut into 40 μm sections which were collected in PBS and used for the subsequent staining procedure. The human brain material was an approximately 1 cm³ deep-frozen block of the neocortex. A small part of the block was immersion-fixated in 4% paraformaldehyde and further treated like the mouse brain material.

Staining was performed by incubating the sections with a solution containing 0.07-7.0 μg/ml of the respective antibody in accordance with the following protocol:

### Materials:

TBST washing solution (Tris Buffered Saline with Tween 20; 10× concentrate; DakoCytomation; S3306 1:10 in Aqua bidest)
0.3% H₂O₂ in methanol
donkey serum (for 6E10, 4G8) or goat serum (for h7C6; Serotec)
monoclonal human 7C6 wt and mut antibody diluted in TBST/1% goat serum
monoclonal mouse antibodies 6E10 (Signet Covance; SIG-39300) and 4G8 (Abcam; Ab1910)
secondary antibody:
   biotinylated donkey-anti-mouse antibody (Jackson Immuno; 715-065-150; diluted 1:500 in TBST/1% donkey serum) for 6E10 and 4G8
   biotinylated goat-anti-human antibody (Abcam; Ab7152, diluted 1:8000 in TBST/1% goat serum) for h7C6
StreptABComplex (DakoCytomation; K 0377)
Peroxidase Substrate Kit diaminobenzidine (=DAB; Vector Laboratories; SK-4100)
SuperFrost Plus microscope slides and coverslips
xylol free embedding medium (Medite; X-tra Kitt)

### Procedure:

Floating sections were transferred into ice-cold 0.3% H₂O₂ and incubated for 30 min.
They were then washed for 5 min. in TBST buffer.
Subsequently, they were incubated with donkey serum/TBST for 20 minutes.
Then, they were incubated with primary antibody for 24 hours at room temperature.
Subsequently, they were washed in TBST buffer for 5 minutes.
They were then incubated with blocking serum from the Vectastain Elite ABC peroxidase kit for 20 minutes.
Subsequently, they were washed in TBST buffer for 5 minutes.
They were then incubated with secondary antibody for 60 minutes at ambient temperature.
Following the above step, the sections were washed in TBST buffer for 5 minutes.
They were then incubated with StreptABComplex for 60 minutes at ambient temperature.
Subsequently, they were washed in TBST buffer for 5 minutes.
The samples were then incubated with DAB from the Vectastain Elite ABC peroxidase kit for 10 minutes.
The sections were then mounted on slides, air-dried, and dehydrated with alcohol and embedded.

Amyloid deposit staining in brain parenchym and vessels was photographed. Then, amyloid plaque staining was additionally quantified by excising approximately 10 randomly selected plaques from the histological images using the ImagePro 5.0 image analysis system and determining their average greyscale value. Optical density values (0%=without material, control=unstained section) were calculated from the greyscale values, and specific staining of the amyloid deposits was obtained by substracting the optical density values from the surrounding background. The differences between the antibodies were tested for statistical significance with ANOVA followed by post-hoc Bonferroni's t-test.

Results of the staining are shown in FIG. 9. In particular, panel a) shows the binding of different antibodies at a concentration of 0.7 μg/ml in transversal section of the neocortices of AD patients or transgenic mice at 19 months of age. Parenchymal Aβ deposits (black arrows) were stained only with 6E10 and 4G8 but not with the h7C6 antibodies. Vascular Aβ deposits (white arrows) were stained only with 6E10 and 4G8 but not with h7C6 antibodies. Panels b)-e) show the binding of different antibodies at a concentration of 0.07-7.0 μg/ml in transversal section of the neocortices of AD patients or old transgenic mice. In particular, binding was only found with ascending concentrations of 6E10 and 4G8, but not with h7C6 antibodies.

Evaluation of brown DAB deposits showed that the Aβ-unselective antibodies 6E10 and 4G8 stained plaques and meningeal vessels, whereas the globulomer selective antibodies h7C6 wt and h7C6mut did not. This finding demonstrates that there is no or markedly less binding of these antibodies to Aβ fibrils or other Aβ species present in the amyloid structures in vivo. This reduced binding should reduce the danger of side effects induced by too quick dissolution of plaques and a subsequent increase in soluble Aβ or neuroinflammation due to the interaction of plaque-bound antibodies with microglia.

### REFERENCE

Dieder Moechars, Ilse Dewachter, Kristin Lorent, Delphine Reversé, Veerle Baekelandt, Asha Naidu, Ina Tesseur, Kurt Spittaels, Chris Van Den Haute, Frederic Checker, Emile Godaux, Barbara Cordel, and Fred Van Leuven (1999), “Early phenotypic changes in transgenic mice that overexpress different mutants of amyloid precursor protein in brain”, J Biol Chem 274:6483-6492.

What is claimed is:

1. A binding protein comprising an antigen binding domain which binds to amyloid-beta (20-42) globulomer, said antigen binding domain comprising at least one CDR comprising an amino acid sequence selected from the group consisting of:
CDR-VH1. X₁-X₂-X₃-X₄-X₅-X₆-X₇ (SEQ ID NO.:5), wherein:
X₁ is T or S;
X₂ is F or Y;
X₃ is Y or A;
X₄ is I or M; and
X₅ is H or S.
CDR-VH2. X₁-X₂-X₃-X₄-X₅-X₆-X₇-X₈-X₉-X₁₀-Xₗₗ-X₁₂-X₁₃-X₁₄-X₁₅-X₁₆-X₁₇ (SEQ ID NO.:6), wherein:
X₁ is M or S;
X₂ is I;
X₃ is G or H;
X₄ is P or N;
X₅ is G or R;
X₆ is S or G;
X₇ is G or T;
X₈ is N or I;
X₉ is T or F;
X₁₀ is Y;
X₁₁ is Y or L;
X₁₂ is N or D;
X₁₃ is E or S;
X₁₄ is M or V;
X₁₅ is F or K;
X₁₆ is K or G; and
X₁₇ is D or is not present.
CDR-VH3. X₁-X₂-X₃-X₄-X₅-X₆-X₇-X₈-X₉-X₁₀-X₁₁-X₁₂-X₁₃ (SEQ ID NO.:7), wherein:
X₁ is A or G;
X₂ is K or R;
X₃ is S;
X₄ is A or N;
X₅ is R or S;
X₆ is A or Y;
X₇ is A;
X₈ is W or M;
X₉ is F or D;
X₁₀ is A or Y; and
X₁₁ is Y or is not present.
CDR-VL1. X₁-X₂-X₃-X₄-X₅-X₆-X₇-X₈-X₉-X₁₀-Xₗₗ-X₁₂-X₁₃-X₁₄-X₁₅-X₁₆ (SEQ ID NO.:8), wherein:
X₁ is R;
X₂ is S
X₃ is S or T;
X₄ is Q;
X₅ is S or T;
X₆ is V or L;
X₇ is V;
X₈ is Q or H;
X₉ is S or R;
X₁₀ is N;
X₁₁ is G;
X₁₂ is N or D;
X₁₃ is T;
X₁₄ is Y;
X₁₅ is N or L and
X₁₆ is E.
CDR-VL2. X₁-X₂-X₃-X₄-X₅-X₆-X₇-X₈ (SEQ ID NO.:9), wherein:
X₁ is K;
X₂ is V;
X₃ is S;
X₄ is N;
X₅ is R;
X₆ is F; and
X₇ is S.
and
CDR-VL3. X₁-X₂-X₃-X₄-X₅-X₆-X₇-X₈-X₉ (SEQ ID NO.:10), wherein:
X₁ is F;
X₂ is Q;
X₃ is G;
X₄ is S;
X₅ is H;
X₆ is V;
X₇ is P;
X₈ is P or Y; and
X₉ is T
wherein said binding protein has a binding affinity to said amyloid beta (20-42) globulomer which is greater than to at least one amyloid beta peptide or protein selected from the group consisting of an amyloid beta (1-42) globulomer, an amyloid beta (12-42) globulomer, an s-amyloid precursor protein, an amyloid beta (1-40) monomer, an amyloid beta (1-42) monomer and an amyloid beta (1-42) fibril.

2. The binding protein according to claim 1, wherein said at least one CDR comprises an amino acid sequence selected from the group consisting of: SEQ ID NO.:11, SEQ ID NO.:12, SEQ ID NO.:13, SEQ ID NO.:14, SEQ ID NO.:15, SEQ ID NO:65, SEQ ID NO.:16, SEQ ID NO.:17, SEQ ID NO.:18, SEQ ID NO.:19, SEQ ID NO.:20 and SEQ ID NO.:21.

3. The binding protein according to claim 1, wherein said binding protein comprises at least 3 CDRs.

4. The binding protein according to claim 3, wherein said at least 3 CDRs are selected from a variable domain CDR set consisting of:

| | |
|---|---|
| VH 5F7 CDR Set | |
| VH 5F7 CDR-H1 | Residues 31-35 of SEQ ID NO.: 1 |
| VH 5F7 CDK-H2 | Residues 50-66 of SEQ ID NO.: 1 |
| VH 5F7 CDR-H3 | Residues 98-108 of SEQ ID NO.: 1 |
| VL 5F7 CDR Set | |
| VL 5F7 CDR-L1 | Residues 24-39 of SEQ ID NO.: 2 |
| VL 5F7 CDR-L2 | Residues 55-61 of SEQ ID NO.: 2 |
| VL 5F7 CDR-L3 | Residues 94-102 of SEQ ID NO.:2 |
| VH 7C6 CDR Set | |
| VH 7C6 CDR-H1 | Residues 31-35 of SEQ ID NO.: 3 |
| VH 7C6 CDR-H2 | Residues 50-65 of SEQ ID NO.: 3 |
| VH 7C6 CDR-H3 | Residues 98-107 of SEQ ID NO.: 3 |
| VL 7C6 CDR Set | |
| VL 7C6 CDR-L1 | Residues 24-39 of SEQ ID NO.: 4 |
| VL 7C6 CDR-L2 | Residues 55-61 of SEQ ID NO.: 4 |
| VL 7C6 CDR-L3 | Residues 94-102 of SEQ ID NO.: 4 |

5. The binding protein according to claim 4, comprising at least two variable domain CDR sets.

6. The binding protein according to claim 5, wherein said at least two variable domain CDR sets are selected from a group consisting of:
VH 7C6 CDR Set & VL 7C6 CDR Set and
VH 5F7 CDR Set & VL 5F7 CDR Set.

7. The binding protein according to claim 3, further comprising a human acceptor framework.

8. The binding protein according to claim 4, further comprising a human acceptor framework.

9. The binding protein according to claim 5, further comprising a human acceptor framework.

10. The binding protein according to claim 6, further comprising a human acceptor framework.

11. The binding protein according to claim 7, wherein said human acceptor framework comprises an amino acid sequence selected from the group consisting of: SEQ ID NO.:48, SEQ ID NO.:49, SEQ ID NO.:50, SEQ ID NO.:51, SEQ ID NO.:52, SEQ ID NO.:53, SEQ ID NO.:54, SEQ ID NO.:55, SEQ ID NO.:56, SEQ ID NO.:57, SEQ ID NO.:58, SEQ ID NO.:59, SEQ ID NO.:60, SEQ ID NO.:61, SEQ ID NO.:62 and SEQ ID NO.:63.

12. The binding protein according to claim 8, wherein said human acceptor framework comprises an amino acid sequence selected from the group consisting of: SEQ ID NO.: 48, SEQ ID NO.:49, SEQ ID NO.:50, SEQ ID NO.:51, SEQ ID NO.:52, SEQ ID NO.:53, SEQ ID NO.:54, SEQ ID NO.:55, SEQ ID NO.:56, SEQ ID NO.:57, SEQ ID NO.:58, SEQ ID NO.:59, SEQ ID NO.:60, SEQ ID NO.:61, SEQ ID NO.:62 and SEQ ID NO.:63.

13. The binding protein according to claim 9, wherein said human acceptor framework comprises an amino acid sequence selected from the group consisting of: SEQ ID NO.:48, SEQ ID NO.:49, SEQ ID NO.:50, SEQ ID NO.:51, SEQ ID NO.:52, SEQ ID NO.:53, SEQ ID NO.:54, SEQ ID NO.:55, SEQ ID NO.:56, SEQ ID NO.:57, SEQ ID NO.:58, SEQ ID NO.:59, SEQ ID NO.:60, SEQ ID NO.:61, SEQ ID NO.:62 and SEQ ID NO.:63.

14. The binding protein according to claim 10, wherein said human acceptor framework comprises a amino acid sequence selected from the group consisting of: SEQ ID NO.:48, SEQ ID NO.:49, SEQ ID NO.:50, SEQ ID NO.:51, SEQ ID NO.:52, SEQ ID NO.:53, SEQ ID NO.:54, SEQ ID NO.:55, SEQ ID NO.:56, SEQ ID NO.:57, SEQ ID NO.:58, SEQ ID NO.:59, SEQ ID NO.:60, SEQ ID NO.:61, SEQ ID NO.:62 and SEQ ID NO.:63.

15. The binding protein according to claim 1, wherein said binding protein comprises at least one variable domain having an amino acid sequence selected from the group consisting of: SEQ ID NO.:1, SEQ ID NO.:2, SEQ ID NO.:3 and SEQ ID NO.:4.

16. The binding protein according to claim 15 wherein said binding protein comprises two variable domains, wherein said two variable domains have amino acid sequences selected from the group consisting of:
SEQ ID NO.:1 & SEQ ID NO.:2, and
SEQ ID NO.:3 & SEQ ID NO.:4.

17. The binding protein according to claim 7, wherein said human acceptor framework comprises at least one Framework Region amino acid substitution at a key residue, said key residue selected from the group consisting of:
a residue adjacent to a CDR;
a glycosylation site residue;
a rare residue;
a residue capable of interacting with Aβ(20-42) globulomer;
a residue capable of interacting with a CDR;
a canonical residue;
a contact residue between heavy chain variable region and light chain variable region;
a residue within a Vernier zone; and
a residue in a region that overlaps between a Chothia-defined variable heavy chain CDR1 and a Kabat-defined first heavy chain framework.

18. The binding protein according to claim 10, wherein said human acceptor framework comprises at least one Framework Region amino acid substitution at a key residue, said key residue selected from the group consisting of:
a residue adjacent to a CDR;
a glycosylation site residue;
a rare residue;
a residue capable of interacting with an Aβ(20-42) globulomer;
a residue capable of interacting with a CDR;
a canonical residue;
a contact residue between heavy chain variable region and light chain variable region;
a residue within a Vernier zone; and
a residue in a region that overlaps between a Chothia-defined variable heavy chain CDR1 and a Kabat-defined first heavy chain framework.

19. The binding protein according to claim 16, wherein said human acceptor framework comprises at least one Framework Region amino acid substitution at a key residue, said key residue selected from the group consisting of:
a residue adjacent to a CDR;
a glycosylation site residue;
a rare residue;
a residue capable of interacting with an Aβ(20-42) globulomer;
a residue capable of interacting with a CDR;
a canonical residue;
a contact residue between heavy chain variable region and light chain variable region;
a residue within a Vernier zone; and
a residue in a region that overlaps between a Chothia-defined variable heavy chain CDR1 and a Kabat-defined first heavy chain framework.

20. The binding protein according to claim 17, wherein the binding protein is a consensus human variable domain.

21. The binding protein according to claim 18, wherein the binding protein is a consensus human variable domain.

22. The binding protein according to claim 19, wherein the binding protein is a consensus human variable domain.

23. The binding protein according to claim 7, wherein said human acceptor framework comprises at least one Framework Region amino acid substitution, wherein the amino acid sequence of the framework is at least 65% identical to the sequence of said human acceptor framework and comprises at least 70 amino acid residues identical to said human acceptor framework.

24. The binding protein according to claim 10, wherein said human acceptor framework comprises at least one Framework Region amino acid substitution, wherein the amino acid sequence of the framework is at least 65% identical to the sequence of said human acceptor framework and comprises at least 70 amino acid residues identical to said human acceptor framework.

25. The binding protein according to claim 16, wherein said human acceptor framework comprises at least one Framework Region amino acid substitution, wherein the amino acid sequence of the framework is at least 65% identical to the sequence of said human acceptor framework and comprises at least 70 amino acid residues identical to said human acceptor framework.

26. The binding protein according to claim 1, wherein said binding protein comprises at least one variable domain having an amino acid sequence selected from the group consisting of: SEQ ID NO.:1, SEQ ID NO.:2, SEQ ID NO.:3 and SEQ ID NO.:4.

27. The binding protein according to claim 26 wherein said binding protein comprises two variable domains, wherein said two variable domains have amino acid sequences selected from the group consisting of: (SEQ ID NO.:1 & SEQ ID NO.:2) and (SEQ ID NO.:3 & SEQ ID NO.:4).

28. The binding protein according to claim 1, wherein the binding protein binds Aβ(20-42) globulomer.

29. The binding protein according to claim 4, wherein the binding protein binds Aβ(20-42) globulomer.

30. The binding protein according to claim 6, wherein the binding protein binds Aβ(20-42) globulomer.

31. The binding protein according to claim 7, wherein the binding protein binds Aβ(20-42) globulomer.

32. The binding protein according to claim 11, wherein the binding protein binds Aβ(20-42) globulomer.

33. The binding protein according to claim 15, wherein the binding protein binds Aβ(20-42) globulomer.

34. The binding protein according to claim 17, wherein the binding protein binds Aβ(20-42) globulomer.

35. The binding protein according to claim 23, wherein the binding protein binds Aβ(20-42) globulomer.

36. The binding protein according to claim 26, wherein the binding protein binds Aβ(20-42) globulomer.

37. The binding protein according to claim 28, wherein the binding protein modulates a biological function of Aβ(20-42) globulomer.

38. The binding protein according to claim 33, wherein the binding protein modulates a biological function of Aβ(20-42) globulomer.

39. The binding protein according to claim 36, wherein the binding protein modulates a biological function of Aβ(20-42) globulomer.

40. The binding protein according to claim 28, wherein the binding protein neutralizes Aβ(20-42) globulomer.

41. The binding protein according to claim 33, wherein the binding protein neutralizes Aβ(20-42) globulomer.

42. The binding protein according to claim 36, wherein the binding protein neutralizes Aβ(20-42) globulomer.

43. The binding protein according to claim 28, wherein said binding protein has a dissociation constant (K_{D}) to said target selected from the group consisting of: at most about 10⁻⁶ M, at most about 10⁻⁷ M, at most about 10⁻⁸ M, at most about 10⁻⁹ M, at most about 10⁻¹⁰ M, at most about 10⁻¹¹ M and at most about 10⁻¹² M.

44. The binding protein according to claim 33, wherein said binding protein has a dissociation constant (K_{D}) to said target selected from the group consisting of: at most about 10⁻⁶ M, at most about 10⁻⁷ M, at most about 10⁻⁸ M, at most about 10⁻⁹ M, at most about 10⁻¹⁰ M, at most about 10⁻¹¹ M and at most about 10⁻¹² M.

45. The binding protein according to claim 35, wherein said binding protein has a dissociation constant (K_{D}) to said target selected from the group consisting of: at most about 10⁻⁶ M, at most about 10⁻⁷ M, at most about 10⁻⁸ M, at most about 10⁻⁹ M, at most about 10⁻¹⁰ M, at most about 10⁻¹¹ M and at most about 10⁻¹² M.

46. The binding protein according to claim 36, wherein said binding protein has a dissociation constant (K_{D}) to said target selected from the group consisting of: at most about 10⁻⁶ M, at most about 10⁻⁷ M, at most about 10⁻⁸ M, at most about 10⁻⁹ M, at most about 10⁻¹⁰ M, at most about 10⁻¹¹ M and at most about 10⁻¹² M.

47. An antibody construct comprising said binding protein of claim 1, said antibody construct further comprising a linker polypeptide or an immunoglobulin constant domain.

48. The antibody construct according to claim 47, wherein said binding protein is selected from the group consisting of:
an immunoglobulin molecule,
a monoclonal antibody,
a chimeric antibody,
a CDR-grafted antibody,
a humanized antibody,
a Fab,
a Fab′,
a F(ab′)2,
a Fv,
a disulfide linked Fv,
a scFv,
a single domain antibody,
a diabody,
a multispecific antibody,
a dual specific antibody, and
a bispecific antibody.

49. The antibody construct according to claim 47, wherein said binding protein comprises a heavy chain immunoglobulin constant domain selected from the group consisting of:
a human IgM constant domain, and
a human IgG1 constant domain,
a human IgG2 constant domain,
a human IgG3 constant domain,
a human IgG4 constant domain,
a human IgE constant domain, and
a human IgA constant domain.

50. The antibody construct according to claim 47, comprising an immunoglobulin constant domain having an amino acid sequence selected from the group consisting of: SEQ ID NO.:38, SEQ ID NO.:39, SEQ ID NO.:40 and SEQ ID NO.:41.

51. An antibody conjugate comprising an antibody construct described in any one of claims 47-50, said antibody conjugate further comprising an agent selected from the group consisting of: an immunoadhesion molecule, an imaging agent, a therapeutic agent, and a cytotoxic agent.

52. The antibody conjugate according to claim 51, wherein said agent is an imaging agent selected from the group consisting of a radiolabel, an enzyme, a fluorescent label, a luminescent label, a bioluminescent label, a magnetic label, and biotin.

53. The antibody conjugate according to claim 52, wherein said radiolabel is selected from the group consisting of: ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

54. The antibody conjugate according to claim 51, wherein said agent is a therapeutic or cytotoxic agent selected from the group consisting of: an anti-metabolite, an alkylating agent, an antibiotic, a growth factor, a cytokine, an anti-angiogenic agent, an anti-mitotic agent, an anthracycline, toxin, and an apoptotic agent.

55. The antibody construct according to claim 49, wherein said binding protein possesses a human glycosylation pattern.

56. The antibody conjugate according to claim 51, wherein said binding protein possesses a human glycosylation pattern.

57. The binding protein according to claim 3, wherein said binding protein exists as a crystal.

58. The antibody construct according to claim 47, wherein said antibody construct exists as a crystal.

59. The antibody conjugate according to claim 51, wherein said antibody construct exists as a crystal.

60. The binding protein according to claim 57, wherein said crystal is a carrier-free pharmaceutical controlled release crystal.

61. The antibody construct according to claim 58, wherein said crystal is a carrier-free pharmaceutical controlled release crystal.

62. The antibody conjugate according to claim 59, wherein said crystal is a carrier-free pharmaceutical controlled release crystal.

63. The binding protein according to claim 57, wherein said binding protein has a greater half life in vivo than the soluble counterpart of said binding protein.

64. The antibody construct according to claim 58, wherein said antibody construct has a greater half life in vivo than the soluble counterpart of said antibody construct.

65. The antibody conjugate according to claim 59, wherein said antibody conjugate has a greater half life in vivo than the soluble counterpart of said antibody conjugate.

66. The binding protein according to claim 57, wherein said binding protein retains biological activity.

67. The antibody construct according to claim 58, wherein said antibody construct retains biological activity.

68. The antibody conjugate according to claim 59, wherein said antibody conjugate retains biological activity.

69. An isolated nucleic acid molecule encoding a binding protein, wherein the amino acid sequence of the variable heavy chain of said binding protein has at least 70% identity to SEQ ID NO.:1.

70. The isolated nucleic acid molecule of claim 69, wherein the amino acid sequence of the light chain of said binding protein has at least 70% identity to SEQ ID NO.:2.

71. An isolated nucleic acid molecule encoding a binding protein, wherein the amino acid sequence of the variable heavy chain of said binding protein has at least 70% identity to SEQ ID NO.:3.

72. The isolated nucleic acid molecule of claim 71, wherein the amino acid sequence of the light chain of said binding protein has at least 70% identity to SEQ ID NO.:4.

73. A vector comprising said isolated nucleic acid molecule of any one of claims 69-72.

74. An isolated host cell comprising said vector of claim 73.

75. A method of producing a protein capable of binding Aβ(20-42) globulomer, comprising culturing said host cell of claim 74 for a time and under conditions sufficient to produce a binding protein capable of binding Aβ(20-42) globulomer.

76. An isolated protein produced according to the method of claim 75.

77. A composition for the release of a binding protein said composition comprising:
(a) a formulation, wherein said formulation comprises a crystal, according to any one of claims 57-59, and an ingredient; and
(b) at least one polymeric carrier.

78. The composition according to claim 77, wherein said polymeric carrier is at least one polymer selected the group consisting of: poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly ((hydroxypropyl)methacrylamide, poly [(organo) phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polyeaccharides, blends, and copolymers thereof.

79. The composition according to claim 77, wherein said ingredient is selected from the group consisting of albumin, sucrose, trehalose, lactitol, gelatin, hydroxypropyl-γ-cyclodextrin, methoxypolyethylene glycol and polyethylene glycol.

80. A method for treating a mammal suspected of having an amyloidosis comprising administering to the mammal said composition of claim 77 in an amount sufficient to effect said treatment.

81. A pharmaceutical composition comprising the binding protein of claim 1, and a pharmaceutically acceptable carrier.

82. The pharmaceutical composition of claim 81 wherein said pharmaceutically acceptable carrier functions as an adjuvant useful to increase the absorption, or dispersion of said binding protein.

83. The pharmaceutical composition of claim 82 wherein said adjuvant is hyaluronidase.

84. The pharmaceutical composition of claim 81 further comprising at least one additional therapeutic agent for treating a disorder in which presence of Aβ(20-42) globulomer is detrimental.

85. The pharmaceutical composition of claim 84, wherein said therapeutic agent is selected from the group consisting of: a monoclonal antibody, a polyclonal antibody, a fragment of a monoclonal antibody, a cholesterinase inhibitor, a partial NMDA receptor blocker, a glycosaminoglycan mimetic, an inhibitor or allosteric modulator of gamma secretase, a luteinizing hormone blockade gonadotropin releasing hormone agonist, a serotinin 5-HT1A receptor antagonist, a chelating agent, a neuronal selective L-type calcium channel blocker, an immunomodulator, an amyloid fibrillogenesis inhibitor or amyloid protein deposition inhibitor, a 5-HT1a receptor antagonist, a PDE4 inhibitor, a histamine agonist, a receptor protein for advanced glycation end products, a PARP stimulator, a serotonin 6 receptor antagonist, a 5-HT4 receptor agonist, a human steroid, a glucose uptake stimulant which enhances neuronal metabolism, a selective CB1 antagonist, a partial agonist at benzodiazepine receptors, an amyloid beta production antagonist or inhibitor, an amyloid beta deposition inhibitor, a NNR alpha-7 partial antagonist, a therapeutic targeting PDE4, a RNA translation inhibitor, a muscarinic agonist, a nerve growth factor receptor agonist, a NGF receptor agonist and a gene therapy modulator.

86. A method for reducing Aβ(20-42) globulomer activity comprising contacting Aβ(20-42) globulomer with the binding protein of claim 1 such that Aβ(20-42) globulomer activity is reduced.

87. A method for reducing human Aβ(20-42) globulomer activity in a human subject suffering from a disorder in which Aβ(20-42) globulomer is detrimental, comprising administering to the human subject the binding protein of claim 1 such that human Aβ(20-42) globulomer activity in the human subject is reduced.

88. A method for treating a subject for a disease or a disorder in which Aβ(20-42) globulomer activity is detrimental by administering to the subject the binding protein of claim 1 in an amount sufficient to effect said treatment.

89. The method of claim 88, wherein said disorder is selected from the group consisting of Alpha1-antitrypsin-deficiency, C1-inhibitor deficiency angioedema, Antithrombin deficiency thromboembolic disease, Kuru, Creutzfeld-Jacob disease/scrapie, Bovine spongiform encephalopathy, Gerstmann-Straussler-Scheinker disease, Fatal familial insomnia, Huntington's disease, Spinocerebellar ataxia, Machado-Joseph atrophy, Dentato-rubro-pallidoluysian atrophy, Frontotemporal dementia, Sickle cell anemia, Unstable hemoglobin inclusion-body hemolysis, Drug-induced inclusion body hemolysis, Parkinson's disease, Systemic AL amyloidosis, Nodular AL amyloidosis, Systemic AA amyloidosis, Prostatic amyloid, Hemodialysis amyloidosis, Hereditary (Icelandic) cerebral angiopathy, Huntington's disease, Familial visceral amyloid, Familial visceral polyneuropathy, Familial visceral amyloidosis, Senile systemic amyloidosis, Familial amyloid neuropathy, Familial cardiac amyloid, Alzheimer's disease, Down's syndrome, Medullary carcinoma thyroid and Type 2 diabetes mellitus (T2DM).

90. A method of treating a patient suffering from a disorder in which Aβ(20-42) globulomer is detrimental comprising the step of administering the binding protein of claim 1 before, concurrent, or after the administration of at least one second agent, wherein said at least one second agent is selected from the group consisting of a monoclonal antibody, a fragment of a monoclonal antibody, a polyclonal antibody, a cholesterinase inhibitor and a partial NMDA receptor blocker.

91. The method of claim 90, wherein said cholesterinase inhibitor is selected from the group consisting of Tacrine, Donepezil, Rivastigmine and Galantamine.

92. The method of claim 90, wherein said partial NMDA receptor blocker is Memantine.

93. The method according to claim 90, wherein said administering to the subject is by at least one mode selected from the group consisting of parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

94. A method of diagnosing Alzheimer's Disease in a patient suspected of having this disease comprising the steps of:
a) isolating a biological sample from said patient;
b) contacting said biological sample with said binding protein of claim 1 for a time and under conditions sufficient for formation of globulomer/binding protein complexes; and
detecting presence of said globulomer/binding protein complexes in said sample, presence of said complexes indicating a diagnosis of Alzheimer's Disease in said patient.

95. A method of diagnosing Alzheimer's Disease in a patient suspected of having this disease comprising the steps of:
a) isolating a biological sample from said patient;
b) contacting said biological sample with said binding protein of claim 1 for a time and under conditions sufficient for the formation of globulomer/binding protein complexes;
c) adding a conjugate to the resulting globulomer/binding protein complexes for a time and under conditions sufficient to allow said conjugate to bind to the bound binding protein, wherein said conjugate comprises an antibody attached to a signal generating compound capable of generating a detectable signal; and
d) detecting the presence of said binding protein which may be present in said biological sample by detecting a signal generated by said signal generating compound, said signal indicating a diagnosis of Alzheimer's Disease in said patient.

96. A method of diagnosing Alzheimer's Disease in a patient suspect of having Alzheimer's Disease comprising the steps of:
a) isolating a biological sample from said patient;
b) contacting said biological sample with anti-binding protein specific for binding protein in said sample for a time and under conditions sufficient to allow for formation of anti-binding protein/binding protein complexes;
c) adding a conjugate to resulting anti-binding protein/binding protein complexes for a time and under conditions sufficient to allow said conjugate to bind to bound binding protein, wherein said conjugate comprises globulomer attached to a signal generating compound capable of generating a detectable signal; and
d) detecting a signal generated by said signal generating compound, said signal indicating a diagnosis of Alzheimer's Disease in said patient.

97. A vaccine comprising said binding protein of claim 1 and a pharmaceutically acceptable adjuvant.

98. A method of detecting a mutant amyloid beta peptide sequence in a patient suspected of having Alzheimer's Disease comprising the steps of:
a) isolating a biological sample from said patient;
b) contacting said biological sample with said binding protein of claim 1 for a time and under conditions sufficient for the formation of mutant antigen/binding protein complexes; and
c) detecting presence of said mutant antigen/binding protein complexes, said complexes indicating said patient has a mutant amyloid beta peptide sequence and thus Alzheimer's Disease.


SEQUENCE LISTING (73 sequences)

SEQ ID NO: 1 - protein, 120 aa, Homo sapiens
EVQLVQSGAEVKKPGASVKVSCKASGYTFTTFYIHWVRQAPGQGLEWIGMIGPGSGNTYYNEMFKDKATLTVDTSTSTAYMELSSLRSEDTAVYYCARAKSARAAWFAYWGQGTLVTVSS

SEQ ID NO: 2 - protein, 113 aa, Homo sapiens
DIVMTQSPLSLPVTPGEPASISCRSSQSVVQSNGNTYLEWYLQKPGQSPQLLIYKVSNRFSGVPDRFSGSGSGTDFTLKISRVEAEDVGVYYCFQGSHVPPTFGGGTKVEIKR

SEQ ID NO: 3 - protein, 118 aa, Homo sapiens
EVKLVESGGGLVKPGGSLRLSCAASGFTFSSYAMSWVRQAPGKGLEWVASIHNRGTIFYLDSVKGRFTISRDNVRNTLYLQMNSLRAEDTAVYYCTRGRSNSYAMDYWGQGTSVTVSS

SEQ ID NO: 4 - protein, 113 aa, Homo sapiens
DVLVTQSPLSLPVTPGEPASISCRSTQTLVHRNGDTYLEWYLQKPGQSPQSLIYKVSNRFSGVPDRFSGSGSGTDFTLKISRVEAEDVGVYYCFQGSHVPYTFGQGTKLEIKR

SEQ ID NO: 5 - protein, 7 aa, Artificial Sequence
  Description of Artificial Sequence Synthetic peptide
XXXXXXX

SEQ ID NO: 6 - protein, 17 aa, Artificial Sequence
  Description of Artificial Sequence Synthetic peptide
XIXXXXXXXYXXXXXXX

SEQ ID NO: 7 - protein, 13 aa, Artificial Sequence
  Description of Artificial Sequence Synthetic peptide
XXSXXXAXXXXXX

SEQ ID NO: 8 - protein, 16 aa, Artificial Sequence
  Description of Artificial Sequence Synthetic peptide
RSXQXXVXXNGXTYXE

SEQ ID NO: 9 - protein, 8 aa, Artificial Sequence
  Description of Artificial Sequence Synthetic peptide
KVSNRFSX

SEQ ID NO: 10 - protein, 9 aa, Artificial Sequence
  Description of Artificial Sequence Synthetic peptide
FQGSHVPXT

SEQ ID NO: 11 - protein, 5 aa, Homo sapiens
TFYIH

SEQ ID NO: 12 - protein, 17 aa, Homo sapiens
MIGPGSGNTYYNEMFKD

SEQ ID NO: 13 - protein, 11 aa, Homo sapiens
AKSARAAWFAY

SEQ ID NO: 14 - protein, 16 aa, Homo sapiens
RSSQSVVQSNGNTYLE

SEQ ID NO: 15 - protein, 7 aa, Homo sapiens
KVSNRFS

SEQ ID NO: 16 - protein, 5 aa, Homo sapiens
SYAMS

SEQ ID NO: 17 - protein, 16 aa, Homo sapiens
SIHNRGTIFYLDSVKG

SEQ ID NO: 18 - protein, 10 aa, Homo sapiens
GRSNSYAMDY

SEQ ID NO: 19 - protein, 16 aa, Homo sapiens
RSTQTLVHRNGDTYLE

SEQ ID NO: 20 - protein, 7 aa, Homo sapiens
KVSNRFS

SEQ ID NO: 21 - protein, 9 aa, Homo sapiens
FQGSHVPYT

SEQ ID NO: 22 - protein, 30 aa, Homo sapiens
QVQLVQSGAEVKKPGASVKVSCKASGYTFT

SEQ ID NO: 23 - protein, 13 aa, Homo sapiens
WRQAPGQGLEWMG

SEQ ID NO: 24 - protein, 32 aa, Homo sapiens
RVTMTRDTSTSTVYMELSSLRSEDTAVYYCAR

SEQ ID NO: 25 - protein, 11 aa, Homo sapiens
WGQGTLVTVSS

SEQ ID NO: 26 - protein, 23 aa, Homo sapiens
DIVMTQSPLSLPVTPGEPASISC

SEQ ID NO: 27 - protein, 15 aa, Homo sapiens
WYLQKPGQSPQLLIY

SEQ ID NO: 28 - protein, 31 aa, Homo sapiens
GVPDRFSSGSGTDFTLKISRVEAEDVGVYYC

SEQ ID NO: 29 - protein, 11 aa, Homo sapiens
FGGGTKVEIKR

SEQ ID NO: 30 - protein, 30 aa, Homo sapiens
EVQLVESGGGLVKPGGSLRLSCAASGFTFS

SEQ ID NO: 31 - protein, 14 aa, Homo sapiens
WVRQAPGKGLEWVS

SEQ ID NO: 32 - protein, 32 aa, Homo sapiens
RFTISRDNAKNSLYLQMNSLRAEDTAVYYCAR

SEQ ID NO: 33 - protein, 11 aa, Homo sapiens
WGQGTLVTVSS

SEQ ID NO: 34 - protein, 23 aa, Homo sapiens
DIVMTQSPLSLPVTPGEPASISC

SEQ ID NO: 35 - protein, 15 aa, Homo sapiens
WYLQKPGQSPQLLIY

SEQ ID NO: 36 - protein, 32 aa, Homo sapiens
GVPDRFSGSGSGTDFTLKISRVEAEDVGVYYC

SEQ ID NO: 37 - protein, 11 aa, Homo sapiens
FGQGTKLEIKR

SEQ ID NO: 38 - protein, 330 aa, Homo sapiens
ASTKGPSVFFLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - DNA, 360 bp, Homo sapiens
gaggtccagctggtgcagtctggagctgaggtgaagaagcctggggcttcagtgaaggtgtcctgcaaggcttctggctacaccttcactaccttctatatacactgggtgaggcaggcgcctggacagggccttgagtggattggaatgattggtcctggaagtggtaatacttactacaatgagatgttcaaggacaaggccacattgactgtagacacatccaccagcacagcctacatggagctcagcagcctcagatctgaggacactgcggtctattactgtgcaagagcaaagtcagctcgggcggcctggtttgcttactggggccaagggactctggtcactgtctcttca

SEQ ID NO: 43 - DNA, 339 bp, Homo sapiens
gatattgtgatgacccaaagtccactctccctgcctgtcactcctggagaaccagcctccatctcttgcagatctagtcagagcgttgtacagagtaatggaaacacctatttagaatggtacctgcagaaaccaggccagtctccacagctcctgatctacaaagtttccaaccgattttctggggtcccagacaggttcagtggcagtggatcagggacagatttcacactcaagatcagcagagtggaggctgaggatgtgggagtttattactgctttcaaggttcacatgttcctcccacgttcggaggggggaccaaggtggaaataaaacgg

SEQ ID NO: 44 - DNA, 354 bp, Homo sapiens
gaagtgaagctggtggagtctgggggaggcttagtgaagcctggagggtccctgagactctcctgtgcagcctctggattcactttcagtagctatgccatgtcttgggttcgccaggctccagggaaggggctagagtgggtcgcgtccattcataatagaggtactatcttctatctagacagtgtgaagggccgattcaccatctccagagataatgtcaggaacaccctgtacctgcaaatgaacagtctgagggctgaggacacggccgtatattactgtacaagaggccggagtaactcctatgctatggactactggggtcaaggaacctcagtcaccgtctcctcg

SEQ ID NO: 45 - DNA, 339 bp, Homo sapiens
gatgttttggtgacccaatctccactctccctgcctgtcacgcctggagaaccagcctccatctcttgccgatctactcagacccttgtacatcgtaatggagacacctatttagaatggtacctgcagaaaccaggccagtctccacagtccctgatctacaaagtttccaaccgattttctggggtcccagacaggttcagcggcagtggatcagggacagatttcacactcaagatcagcagagtggaggctgaggatgtgggagtttattactgctttcaaggttcacatgttccgtacacgttcggacaggggaccaagctggaaataaaacgg

SEQ ID NO: 46 - protein, 42 aa, Homo sapiens [sequence omitted]

SEQ ID NO: 47 - protein, 40 aa, Homo sapiens
DAEFRHDSGYEVHHQKLVFFAEDVGSNKGAIIGLMVGGVV

SEQ ID NO: 48 - protein, 30 aa, Homo sapiens; identical to SEQ ID NO: 22

SEQ ID NO: 49 - protein, 14 aa, Homo sapiens
WVRQAPGQGLEWMG

SEQ ID NO: 50 - protein, 32 aa, Homo sapiens; identical to SEQ ID NO: 24

SEQ ID NO: 51 - protein, 11 aa, Homo sapiens
WGQGTLVTVSS

SEQ ID NO: 52 - protein, 30 aa, Homo sapiens; identical to SEQ ID NO: 30

SEQ ID NO: 53 - protein, 14 aa, Homo sapiens
WVRQAPGKGLEWVS

SEQ ID NO: 54 - protein, 32 aa, Homo sapiens; identical to SEQ ID NO: 32

SEQ ID NO: 55 - protein, 11 aa, Homo sapiens
WGQGTLVTVSS

SEQ ID NO: 56 - protein, 23 aa, Homo sapiens
DIVMTQSPLSLPVTPGEPASISC

SEQ ID NO: 57 - protein, 15 aa, Homo sapiens
WYLQKPGQSPQLLIY

SEQ ID NO: 58 - protein, 31 aa, Homo sapiens; identical to SEQ ID NO: 28

SEQ ID NO: 59 - protein, 11 aa, Homo sapiens
FGGGTKVEIKR

SEQ ID NO: 60 - protein, 23 aa, Homo sapiens
DIVMTQSPLSLPVTPGEPASISC

SEQ ID NO: 61 - protein, 15 aa, Homo sapiens
WYLQKPGQSPQLLIY

SEQ ID NO: 62 - protein, 32 aa, Homo sapiens; identical to SEQ ID NO: 36

SEQ ID NO: 63 - protein, 11 aa, Homo sapiens
FGQGTKLEIKR

SEQ ID NO: 64 - protein, 43 aa, Homo sapiens
DAEFRHDSGYEVHHQKLVFFAEDVGSNKGAIIGLMVGGVVIAT

SEQ ID NO: 65 - protein, 9 aa, Homo sapiens
FQGSHVPPT

SEQ ID NO: 66 - protein, 31 aa, Homo sapiens
VHHQKLVFFAEDVGSNKGAIIGLMVGGVVIA

SEQ ID NO: 67 - protein, 23 aa, Homo sapiens
FAEDVGSNKGAIIGLMVGGVVIA

SEQ ID NO: 68 - protein, 120 aa, Homo sapiens
QVQLKQSGAELVRPGTSVKMSCKASGYTFTTFYIHWVKQRPGQGLEWIGMIGPGSGNTYYNEMFKDKATLTVDTSSSTAYMQLSSLTSEDSAVYFCARAKSARAAWFAYWGQGTLVTVSA

SEQ ID NO: 69 - protein, 120 aa, Homo sapiens
QVQLVQSGAEVKKPGASVKVSCKASGYTFTTFYIHWVRQAPGQGLEWIGMIGPGSGNTYYNEMFKDKATLTVDTSTSTAYMELSSLRSEDTAVYYCARAKSARAAWFAYWGQGTLVTVSS

SEQ ID NO: 70 - protein, 87 aa, Homo sapiens
QVQLVQSGAEVKKPGASVKVSCKASGYTFTWVRQAPGQGLEWMGRVTITRDTSASTAYMELSSLRSEDTAVYYCARWGQGTLVTVSS

SEQ ID NO: 71 - protein, 113 aa, Homo sapiens
DVLMTQTPLSLPVSLGDQASISCRSSQSVVQSNGNTYLEWYLQKPGQSPKLLIYKVSNRFSGVPDRFSGSGSGTDFTLKISRVEAEDLGVYYCFQGSHVPPTFGGGTKLEIKR

SEQ ID NO: 72 - protein, 113 aa, Homo sapiens
DIVMTQTPLSLPVTPGEPASISCRSSQSVVQSNGNTYLEWYLQKPGQSPQLLIYKVSNRFSGVPDRFSGSGSGTDFTLKISRVEAEDVGVYYCFQGSHVPPTFGGGTKVEIKR

SEQ ID NO: 73 - protein, 81 aa, Homo sapiens
ELVMTQSPLSLPVTPGEPASISCWYLQKPGQSPQLLIYGVPDRFSGSGSGTDFTLKISRVEAEDVGVYYCFGGGTKVEIKR